SEQUENCE LISTING (39 sequences)

SEQ ID NO: 1 - protein, 319 aa, Artificial Sequence
  Synthetic accA, pNH241
MSLNFLDFEQPIAELEAKIDSLTAVSRQDEKLDINIDEEVHRLREKSVELTRKIFADLGAWQIAQLARHPQRPYTLDYVRLAFDEFDELAGDRAYADDKAIVGGIARLDGRPVMIIGHQKGRETKEKIRRNFGMPAPEGYRKALRLMQMAERFKMPIITFIDTPGAYPGVGAEERGQSEAIARNLREMSRLGVPVVCTVIGEGGSGGALAIGVGDKVNMLQYSTYSVISPEGCASILWKSADKAPLAAEAMGIIAPRLKELKLIDSIIPEPLGGAHRNPEAMAASLKAQLLADLADLDVLSTEDLKNRRYQRLMSYGYA

SEQ ID NO: 2 - protein, 156 aa, Artificial Sequence
  Synthetic accB, pNH241
MDIRKIKKLIELVEESGISELEISEGEESVRISRAAPAASFPVMQQAYAAPMMQQPAQSNAAAPATVPSMEAPAAAEISGHIVRSPMVGTFYRTPSPDAKAFIEVGQKVNVGDTLCIVEAMKMMNQIEADKSGTVKAILVESGQPVEFDEPLVVIE

SEQ ID NO: 3 - protein, 449 aa, Artificial Sequence
  Synthetic accC, pNH241
MLDKIVIANRGEIALRILRACKELGIKTVAVHSSADRDLKHVLLADETVCIGPAPSVKSYLNIPAIISAAEITGAVAIHPGYGFLSENANFAEQVERSGFIFIGPKAETIRLMGDKVSAIAAMKKAGVPCVPGSDGPLGDDMDKNRAIAKRIGYPVIIKASGGGGGRGMRVVRGDAELAQSISMTRAEAKAAFSNDMVYMEKYLENPRHVEIQVLADGQGNAIYLAERDCSMQRRHQKVVEEAPAPGITPELRRYIGERCAKACVDIGYRGAGTFEFLFENGEFYFIEMNTRIQVEHPVTEMITGVDLIKEQLRIAAGQPLSIKQEEVHVRGHAVECRINAEDPNTFLPSPGKITRFHAPGGFGVRWESHIYAGYTVPPYYDSMIGKLICYGENRDVAIARMKNALQELIIDGIKTNVDLQIRIMNDENFQHGGTNIHYLEKKLGLQEK

SEQ ID NO: 4 - protein, 304 aa, Artificial Sequence
  Synthetic accD, pNH241
MSWIERIKSNITPTRKASIPEGVWTKCDSCGQVLYRAELERNLEVCPKCDHHMRMTARNRLHSLLDEGSLVELGSELEPKDVLKFRDSKKYKDRLASAQKETGEKDALVVMKGTLYGMPVVAAAFEFAFMGGSMGSVVGARFVRAVEQALEDNCPLICFSASGGARMQEALMSLMQMAKTSAALAKMQERGLPYISVLTDPTMGGVSASFAMLGDLNIAEPKALIGFAGPRVIEQTVREKLPPGFQRSEFLIEKGAIDMIVRRPEMRLKLASILAKLMNLPAPNPEAPREGVVVPPVPDQEPEA

SEQ ID NO: 5 - protein, 550 aa, Artificial Sequence
  Synthetic mcrN, pNH243
MSGTGRLAGKIALITGGAGNIGSELTRRFLAEGATVIISGRNRAKLTALAERMQAEAGVPAKRIDLEVMDGSDPVAVRAGIEAIVARHGQIDILVNNAGSAGAQRRLAEIPLTEAELGPGAEETLHASIANLLGMGWHLMRIAAPHMPVGSAVINVSTIFSRAEYYGRIPYVTPKAALNALSQLAARELGARGIRVNTIFPGPIESDRIRTVFQRMDQLKGRPEGDTAHHFLNTMRLCRANDQGALERRFPSVGDVADAAVFLASAESAALSGETIEVTHGMELPACSETSLLARTDLRTIDASGRTTLICAGDQIEEVMALTGMLRTCGSEVIIGFRSAAALAQFEQAVNESRRLAGADFTPPIALPLDPRDPATIDAVFDWGAGENTGGIHAAVILPATSHEPAPCVIEVDDERVLNFLADEITGTIVIASRLARYWQSQRLTPGARARGPRVIFLSNGADQNGNVYGRIQSAAIGQLIRVWRHEAELDYQRASAAGDHVLPPVWANQIVRFANRSLEGLEFACAWTAQLLHSQRHINEITLNIPANI

SEQ ID NO: 6 - protein, 671 aa, Artificial Sequence
  Synthetic mcrC3, pNH243
MSATTGARSASVGWAESLIGLHLGKVALITGGSAGIGGQIGRLLALSGARVMLAARDRHKLEQMQAMIQSELAEVGYTDVEDRVHIAPGCDVSSEAQLADLVERTLSAFGTVDYLINNAGIAGVEEMVIDMPVEGWRHTLFANLISNYSLMRKLAPLMKKQGSGYILNVSSYFGGEKDAAIPYPNRADYAVSKAGQRAMAEVFARFLGPEIQINAIAPGPVEGDRLRGTGERPGLFARRARLILENKRLNELHAALIAAARTDERSMHELVELLLPNDVAALEQNPAAPTALRELARRFRSEGDPAASSSSALLNRSIAAKLLARLHNGGYVLPADIFANLPNPPDPFFTRAQIDREARKVRDGIMGMLYLQRMPTEFDVAMATVYYLADRVVSGETFHPSGGLRYERTPTGGELFGLPSPERLAELVGSTVYLIGEHLTEHLNLLARAYLERYGARQVVMIVETETGAETMRRLLHDHVEAGRLMTIVAGDQIEAAIDQAITRYGRPGPVVCTPFRPLPTVPLVGRKDSDWSTVLSEAEFAELCEHQLTHHFRVARWIALSDGARLALVTPETTATSTTEQFALANFIKTTLHAFTATIGVESERTAQRILINQVDLTRRARAEEPRDPHERQQELERFIEAVLLVTAPLPPEADTRYAGRIHRGRAITV

SEQ ID NO: 7 - protein, 671 aa, Artificial Sequence
  Synthetic mcrC0
MSATTGARSASVGWAESLIGLHLGKVALITGGSAGIGGQIGRLLALSGARVMLAARDRHKLEQMQAMIQSELAEVGYTDVEDRVHIAPGCDVSSEAQLADLVERTLSAFGTVDYLINNAGIAGVEEMVIDMPVEGWRHTLFANLISNYSLMRKLAPLMKKQGSGYILNVSSYFGGEKDAAIPYPNRADYAVSKAGQRAMAEVFARFLGPEIQINAIAPGPVEGDRLRGTGERPGLFARRARLILENKRLNELHAALIAAARTDERSMHELVELLLPNDVAALEQNPAAPTALRELARRFRSEGDPAASSSSALLNRSIAAKLLARLHNGGYVLPADIFANLPNPPDPFFTRAQIDREARKVRDGIMGMLYLQRMPTEFDVAMATVYYLADRNVSGETFHPSGGLRYERTPTGGELFGLPSPERLAELVGSTVYLIGEHLTEHLNLLARAYLERYGARQVVMIVETETGAETMRRLLHDHVEAGRLMTIVAGDQIEAAIDQAITRYGRPGPVVCTPFRPLPTVPLVGRKDSDWSTVLSEAEFAELCEHQLTHHFRVARKIALSDGASLALVTPETTATSTTEQFALANFIKTTLHAFTATIGVESERTAQRILINQVDLTRRARAEEPRDPHERQQELERFIEAVLLVTAPLPPEADTRYAGRIHRGRAITV

SEQ ID NO: 8 - protein, 1220 aa, Artificial Sequence
  Synthetic mcr
MSGTGRLAGKIALITGGAGNIGSELTRRFLAEGATVIISGRNRAKLTALAERMQAEAGVPAKRIDLEVMDGSDPVAVRAGIEAIVARHGQIDILVNNAGSAGAQRRLAEIPLTEAELGPGAEETLHASIANLLGMGWHLMRIAAPHMPVGSAVINVSTIFSRAEYYGRIPYVTPKAALNALSQLAARELGARGIRVNTIFPGPIESDRIRTVFQRMDQLKGRPEGDTAHHFLNTMRLCRANDQGALERRFPSVGDVADAAVFLASAESAALSGETIEVTHGMELPACSETSLLARTDLRTIDASGRTTLICAGDQIEEVMALTGMLRTCGSEVIIGFRSAAALAQFEQAVNESRRLAGADFTPPIALPLDPRDPATIDAVFDWGAGENTGGIHAAVILPATSHEPAPCVIEVDDERVLNFLADEITGTIVIASRLARYWQSQRLTPGARARGPRVIFLSNGADQNGNVYGRIQSAAIGQLIRVWRHEAELDYQRASAAGDHVLPPVWANQIVRFANRSLEGLEFACAWTAQLLHSQRHINEITLNIPANISATTGARSASVGWAESLIGLHLGKVALITGGSAGIGGQIGRLLALSGARVMLAARDRHKLEQMQAMIQSELAEVGYTDVEDRVHIAPGCDVSSEAQLADLVERTLSAFGTVDYLINNAGIAGVEEMVIDMPVEGWRHTLFANLISNYSLMRKLAPLMKKQGSGYILNVSSYFGGEKDAAIPYPNRADYAVSKAGQRAMAEVFARFLGPEIQINAIAPGPVEGDRLRGTGERPGLFARRARLILENKRLNELHAALIAAARTDERSMHELVELLLPNDVAALEQNPAAPTALRELARRFRSEGDPAASSSSALLNRSIAAKLLARLHNGGYVLPADIFANLPNPPDPFFTRAQIDREARKVRDGIMGMLYLQRMPTEFDVAMATVYYLADRNVSGETFHPSGGLRYERTPTGGELFGLPSPERLAELVGSTVYLIGEHLTEHLNLLARAYLERYGARQVVMIVETETGAETMRRLLHDHVEAGRLMTIVAGDQIEAAIDQAITRYGRPGPVVCTPFRPLPTVPLVGRKDSDWSTVLSEAEFAELCEHQLTHHFRVARKIALSDGASLALVTPETTATSTTEQFALANFIKTTLHAFTATIGVESERTAQRILINQVDLTRRARAEEPRDPHERQQELERFIEAVLLVTAPLPPEADTRYAGRIHRGRAITV

SEQ ID NO: 9 - protein, 527 aa, Artificial Sequence
  Synthetic mmoX, pNH265
MALSTATKAATDALAANRAPTSVNAQEVHRWLQSFNWDFKNNRTKYATKYKMANETKEQFKLIAKEYARMEAVKDERQFGSLQDALTRLNAGVRVHPKWNETMKVVSNFLEVGEYNAIAATGMLWDSAQAAEQKNGYLAQVLDEIRHTHQCAYVNYYFAKNGQDPAGHNDARRTRTIGPLWKGMKRVFSDGFISGDAVECSLNLQLVGEACFTNPLIVAVTEWAAANGDEITPTVFLSIETDELRHMANGYQTVVSIANDPASAKYLNTDLNNAFWTQQKYFTPVLGMLFEYGSKFKVEPWVKTWNRWVYEDWGGIWIGRLGKYGVESPRSLKDAKQDAYWAHHDLYLLAYALWPTGFFRLALPDQEEMEWFEANYPGWYDHYGKIYEEWRARGCEDPSSGFIPLMWFIENNHPIYIDRVSQVPFCPSLAKGASTLRVHEYNGQMHTFSDQWGERMWLAEPERYECQNIFEQYEGRELSEVIAELHGLRSDGKTLIAQPHVRGDKLWTLDDIKRLNCVFKNPVKAFN

SEQ ID NO: 10 - protein, 389 aa, Artificial Sequence
  Synthetic mmoY, pNH265
MSMLGERRRGLTDPEMAAVILKALPEAPLDGNNKMGYFVTPRWKRLTEYEALTVYAQPNADWIAGGLDWGDWTQKFHGGRPSWGNETTELRTVDWFKHRDPLRRWHAPYVKDKAEEWRYTDRFLQGYSADGQIRAMNPTWRDEFINRYWGAFLFNEYGLFNAHSQGAREALSDVTRVSLAFWGFDKIDIAQMIQLERGFLAKIVPGFDESTAVPKAEWTNGEVYKSARLAVEGLWQEVFDWNESAFSVHAVYDALFGQFVRREFFQRLAPRFGDNLTPFFINQAQTYFQIAKQGVQDLYYNCLGDDPEFSDYNRTVMRNWTGKWLEPTIAALRDFMGLFAKLPAGTTDKEEITASLYRVVDDWIEDYASRIDFKADRDQIVKAVLAGLK

SEQ ID NO: 11 - protein, 141 aa, Artificial Sequence
  Synthetic mmoB, pNH265
MSVNSNAYDAGIMGLKGKDFADQFFADENQVVHESDTVVLVLKKSDEINTFIEEILLTDYKKNVNPTVNVEDRAGYWWIKANGKIEVDCDEISELLGRQFNVYDFLVDVSSTIGRAYTLGNKFTITSELMGLDRKLEDYHA

SEQ ID NO: 12 - protein, 170 aa, Artificial Sequence
  Synthetic mmoZ, pNH265
MAKLGIHSNDTRDAWVNKIAQLNTLEKAAEMLKQFRMDHTTPFRNSYELDNDYLWIEAKLEEKVAVLKARAFNEVDFRHKTAFGEDAKSVLDGTVAKMNAAKDKWEAEKIHIGFRQAYKPPIMPVNYFLDGERQLGTRLMELRNLNYYDTPLEELRKQRGVRVVHLQSPH

SEQ ID NO: 13 - protein, 348 aa, Artificial Sequence
  Synthetic mmoC, pNH265
MQRVHTITAVTEDGESLRFECRSDEDVITAALRQNIFLMSSCREGGCATCKALCSEGDYDLKGCSVQALPPEEEEEGLVLLCRTYPKTDLEIELPYTHCRISFGEVGSFEAEVVGLNWVSSNTVQFLLQKRPDECGNRGVKFEPGQFMDLTIPGTDVSRSYSPANLPNPEGRLEFLIRVLPEGRFSDYLRNDARVGQVLSVKGPLGVFGLKERGMAPRYFVAGGTGLAPVVSMVRQMQEWTAPNETRIYFGVNTEPELFYIDELKSLERSMRNLTVKACVWHPSGDWEGEQGSPIDALREDLESSDANPDIYLCGPPGMIDAACELVRSRGIPGEQVFFEKFLPSGAA

SEQ ID NO: 14 - protein, 103 aa, Artificial Sequence
  Synthetic mmoD, pNH265
MVESAFQPFSGDADEWFEEPRPQAGFFPSADWHLLKRDETYAAYAKDLDFMWRWVIVREERIVQEGCSISLESSIRAVTHVLNYFGMTEQRAPAEDRTGGVQH

SEQ ID NO: 15 - protein, 559 aa, Artificial Sequence
  Synthetic groEL-2, pNH265
MAKEVVYRGSARQRMMQGIEILARAAIPTLGATGPSVMIQHRADGLPPISTRDGVTVANSIVLKDRVANLGARLLRDVAGTMSREAGDGTTTAIVLARHIAREMFKSLAVGADPIALKRGIDRAVARVSEDIGARAWRGDKESVILGVAAVATKGEPGVGRLLLEALDAVGVHGAVSIELGQRREDLLDVVDGYRWEKGYLSPYFVTDRARELAELEDVYLLMTDREVVDFIDLVPLLEAVTEAGGSLLIAADRVHEKALAGLLLNHVRGVFKAVAVTAPGFGDKRPNRLLDLAALTGGRAVLEAQGDRLDRVTLADLGRVRRAVVSADDTALLGIPGTEASRARLEGLRLEAEQYRALKPGQGSATGRLHELEEIEARIVGLSGKSAVYRVGGVTDVEMKERMVRIENAYRSVVSALEEGVLPGGGVGFLGSMPVLAELEARDADEARGIGIVRSALTEPLRIIGENSGLSGEAVVAKVMDHANPGWGYDQESGSFCDLHARGIWDAAKVLRLALEKAASVAGTFLTTEAVVLEIPDTDAFAGFSAEWAAATREDPRV

SEQ ID NO: 16 - protein, 96 aa, Artificial Sequence
  Synthetic groES_mc, pNH265
VKIRPLHDRVIIKRLEEERTSAGGIVIPDSAAEKPMRGEILAVGNGKVLDNGEVRALQVKVGDKVLFGKYAGTEVKVDGEDVVVMREDDILAVLES

SEQ ID NO: 17 - protein, 97 aa, Artificial Sequence [sequence omitted]

SEQ ID NO: 18 - protein, 542 aa, Artificial Sequence
  Synthetic groEL_ec, pNH265
MAAKDVKFGNDARVKMLRGVNVLADAVKVTLGPKGRNVVLDKSFGAPTITKDGVSVAREIELEDKFENMGAQMVKEVASKANDAAGDGTTTATVLAQAIITEGLKAVAAGMNPMDLKRGIDKAVTAAVEELKALSVPCSDSKAIAQVGTISANSDETVGKLIAEAMDKVGKEGVITVEDGTGLQDELDVVEGMQFDRGYLSPYFINKPETGAVELESPFILLADKKISNIREMLPVLEAVAKAGKPLLIIAEDVEGEALATLVVNTMRGIVKVAAVKAPGFGDRRKAMLQDIATLTGGTVISEEIGMELEKATLEDLGQAKRVVINKDTTTIIDGVGEEAAIQGRVAQIRQQIEEATSDYDREKLQERVAKLAGGVAVIKVGAATEVEMKEKKARVEDALHATRAAVEEGVVAGGGVALIRVASKLADLRGQNEDQNVGIKVALRAMEAPLRQIVLNCGEEPSVVANTVKGGDGNYGYNAATEEYGNMIDMGILDPTKVTRSALQYAASVAGLMITTECMVTDLPKNDAADLGAAGGMGGMM

SEQ ID NO: 19 - protein, 211 aa, Artificial Sequence
  Synthetic HPS, pLC130
MELQLALDLVNIEEAKQVVAEVQEYVDIVEIGTPVIKIWGLQAVKAVKDAFPHLQVLADMKTMDAAAYEVAKAAEHGADIVTILAAAEDVSIKGAVEEAKKLGKKILVDMIAVKNLEERAKQVDEMGVDYICVHAGYDLQAVGKNPLDDLKRIKAVVKNAKTAIAGGIKLETLPEVIKAEPDLVIVGGGIANQTDKKAAAEKINKLVKQGL

SEQ ID NO: 20 - protein, 187 aa, Artificial Sequence
  Synthetic PHI, pLC130
MISMLTTEFLAEIVKELNSSVNQIADEEAEALVNGILQSKKVFVAGAGRSGFMAKSFAMRMMHMGIDAYVVGETVTPNYEKEDILIIGSGSGETKSLVSMAQKAKSIGGTIAAVTINPESTIGQLADIVIKMPGSPKDKSEARETIQPMGSLFEQTLLLFYDAVILRFMEKKGLDTKTMYGRHANLE

SEQ ID NO: 21 - protein, 385 aa, Artificial Sequence
  Synthetic mdh2_Bm, pLC130
MTNTQSAFFMPSVNLFGAGSVNEVGTRLADLGVKKALLVTDAGLHGLGLSEKISSIIRAAGVEVSIFPKAEPNPTDKNVAEGLEAYNAENCDSIVTLGGGSSHDAGKAIALVAANGGKIHDYEGVDVSKEPMVPLIAINTTAGTGSELTKFTIITDTERKVKMAIVDKHVTPTLSINDPELMVGMPPSLTAATGLDALTHAIEAYVSTGATPITDALAIQAIKIISKYLPRAVANGKDIEAREQMAFAQSLAGMAFNNAGLGYVHAIAHQLGGFYNFPHGVCNAVLLPYVCRFNLISKVERYAEIAAFLGENVDGLSTYDAAEKAIKAIERMAKDLNIPKGFKELGAKEEDIETLAKNAMKDACALTNPRKPKLEEVIQIIKNAM

SEQ ID NO: 22 - protein, 211 aa, Artificial Sequence; identical to SEQ ID NO: 19
  Synthetic HPS, pLC158

SEQ ID NO: 23 - protein, 187 aa, Artificial Sequence; identical to SEQ ID NO: 20
  Synthetic PHI, pLC158

SEQ ID NO: 24 - protein, 345 aa, Artificial Sequence
  Synthetic adhA_CG, pLC158
MTTAAPQEFTAAVVEKFGHDVTVKDIDLPKPGPHQALVKVLTSGICHTDLHALEGDWPVKPEPPFVPGHEGVGEVVELGPGEHDVKVGDIVGNAWLWSACGTCEYCITGRETQCNEAEYGGYTQNGSFGQYMLVDTRYAARIPDGVDYLEAAPILCAGVTVYKALKVSETRPGQFMVISGVGGLGHIAVQYAAAMGMRVIAVDIADDKLELARKHGAEFTVNARNEDSGEAVQKYTNGGAHGVLVTAVHEAAFGQALDMARRAGTIVFNGLPPGEFPASVFNIVFKGLTIRGSLVGTRQDLAEALDFFARGLIKPTVSECSLDEVNGVLDRMRNGKIDGRVAIRY

SEQ ID NO: 25 - protein, 385 aa, Artificial Sequence; identical to SEQ ID NO: 21
  Synthetic mdh2_Bm, pBZ27

SEQ ID NO: 26 - protein, 382 aa, Artificial Sequence
  Synthetic mdh_Bm, pBZ27
MTTNFFIPPASVIGRGAVKEVGTRLKQIGAKKALIVTDAFLHSTGLSEEVAKNIREAGVDVAIFPKAQPDPADTQVHEGVDVFKQENCDSLVSIGGGSSHDTAKAIGLVAANGGRINDYQGVNSVEKPVVPVVAITTTAGTGSETTSLAVITDSARKVKMPVIDEKITPTVAIVDPELMVKKPAGLTIATGMDALSHAIEAYVAKGATPVTDAFAIQAMKLINEYLPKAVANGEDIEAREKMAYAQYMAGVAFNNGGLGLVHSISHQVGGVYKLQHGICNSVNMPHVCAFNLIAKTERFAHIAELLGENVAGLSTAAAAERAIVALERINKSFGIPSGYAEMGVKEEDIELLAKNAYEDVCTQSNPRVPTVQDIAQIIKNAM

SEQ ID NO: 27 - protein, 211 aa, Artificial Sequence; identical to SEQ ID NO: 19
  Synthetic HPS, pBZ27

SEQ ID NO: 28 - protein, 187 aa, Artificial Sequence; identical to SEQ ID NO: 20
  Synthetic PHI, pBZ27

SEQ ID NO: 29 - protein, 214 aa, Artificial Sequence
  Synthetic rpeP, pBZ27
MIKIAPSILSANFARLEEEIKDVERGGADYIHVDVMDGHFVPNITIGPLIVEAIRPVTNLPLDVHLMIENPDQYIGTFAKAGADILSVHVEACTHLHRTIQYIKSEGIKAGVVLNPHTPVSMIEHVIEDVDLVLLMTVNPGFGGQSFIHSVLPKIKQVANIVKEKNLQVEIEVDGGVNPETAKLCVEAGANVLVAGSAIYNQEDRSQAIAKIRN

SEQ ID NO: 30 - protein, 320 aa, Artificial Sequence
  Synthetic glpXP, pBZ27
MRELKSEKRVQSLAMEFLSVAQQAALASYPWIGKGNKNEVDRAGTEAMRNRLNLIDMSGLIVIGEGEMDEAPMLYIGEELGTGKGPQLDIAVDPVDGTGLMAKGMDNSIAVIAASTRGSLLHAPDMYMEKIAVGPKAKGCVNLDASLTENMKSVAKALGKDLRELTVMIQDRPRHDHLIQQVRDVGARLKLFSDGDVTRAIGTALEEVDVDILVGTGGAPEGVIAATALKCLGGDFQGRLAPQNEEEFDRCITMGITDPRKIFTIDEIVKSDDCFFVATGITDGLLINGIRKKEDGLMQTHSFLTIGGSSVKYQFIEAYH

SEQ ID NO: 31 - protein, 284 aa, Artificial Sequence
  Synthetic fbaP, pBZ27
MPLVSMKDMLNHGKENGYAVGQFNINNLEFGQAILQAAEEEKSPVIIGVSVGAANYMGGFKLIVDMVKSLMDSYNVTVPVAIHLDHGPSLEKCVQAIHAGFTSVMIDGSHLPLEENIELTKRVVEIAHSVGVSVEAELGRIGGQEDDVVAESFYAIPSECEQLVRETGVDCFAPALGSVHGPYKGEPKLGFDRMEEIMKLTGVPLVLHGGTGIPTKDIQKAISLGTAKINVNTESQIAATKAVREVLNNDAKLFDPRKFLAPAREAIKETIKGKMREFGSSGKA

SEQ ID NO: 32 - protein, 667 aa, Artificial Sequence
  Synthetic tktP, pBZ27
VLQQKIDIDQLSIQTIRTLSIDAIEKVGSGHPGMPMGAAPMAYTLWTKFMNYNPSNPNWFNRDRFVLSAGHGSMLLYSLLHLTGYDLSLEDLKNFRQWGSKTPGHPEFGHTPGVDATTGPLGQGIAMAVGMAMAERHLASKYNRYKFNIIDHYTYSICGDGDLMEGVSAEAASLAGHLKLGRLIVLYDSNDISLDGDLHMSFSESVQDRFKAYGWQVLRVEDGNDIDSIAKAIAEAKNNEDQPTLIEVKTIIGYGSPNKGGKSDAHGSPLGKEEIKLVKEHYNWKYDEDFYIPEEVKEYFRELKEAAEKKEQAWNELFAQYKEAYPALAKELEQAINGELPEGWDADVPVYRVGEDKLATRSSSGAVLNALAKNVPQLLGGSADLASSNKTLLKGEANFSATDYSGRNIWFGVREFGMGAAVNGMALHGGVKVFGATFFVFSDYLRPAIRLSALMKLPVIYVFTHDSVAVGEDGPTHEPIEQLASLRAMPGISTIRPADGNETAAAWKLALESKDEPTALILSRQDLPTLVDSEKAYEGVKKGAYVISEAKGEVAGLLLASGSEVALAVEAQAALEKEGIYVSVVSMPSWDRFEKQSDAYKESVLPKNVKARLGIEMGASLGWSKYVGDNGNVLAIDQFGSSAPGDKIIEEYGFTVENVVSHFKKLL

SEQ ID NO: 33 - protein, 322 aa, Artificial Sequence
  Synthetic pfkP, pBZ27
MNKIAVLTSGGDAPGMNAAIRAVVRRGIFKGLDVYGVKNGYKGLMNGNFVSMNLGSVGDIIHRGGTILQTTRCKEFKTAEGQQQALAQLKKEGIDGLIVIGGDGTFEGARKLTAQEFPTIGIPATIDNDIAGTEYTIGFDTAVNTAVEAIDKIRDTAASHDRIYVVEVMGRNAGDIALWAGMCAGAESIIIPEADHDVEDVIDRIKQGYQRGKTHSIIVVAEGAFNGVGAIEIGRAIKEKTGFDTKVTILGHIQRGGSPSAYDRMMSSQMGAKAVDLLVEGKKGLMVGLKNGQLIHTPFEEAAKDKHTVDLSIYHLARSLSL

SEQ ID NO: 34 - DNA, 9600 bp, Artificial Sequence
  Synthetic pNH241
taatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctagattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttgcagccgaacgacctagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggatctcgagggtgttttcacgagcaattgaccaacaaggacaggaggcctaatgagctggattgaacgaattaaaagcaacattactcccacccgcaaggcgagcattcctgaaggggtgtggactaagtgtgatagctgcggtcaggttttataccgcgctgagctggaacgtaatcttgaggtctgtccgaagtgtgaccatcacatgcgtatgacagcgcgtaatcgcctgcatagcctgttagatgaaggaagccttgtggagctgggtagcgagcttgagccgaaagatgtgctgaagtttcgtgactccaagaagtataaagaccgtctggcatctgcgcagaaagaaaccggcgaaaaagatgcgctggtggtgatgaaaggcactctgtatggaatgccggttgtcgctgcggcattcgagttcgcctttatgggcggttcaatggggtctgttgtgggtgcacgtttcgtgcgtgccgttgagcaggcgctggaagataactgcccgctgatctgcttctccgcctctggtggcgcacgtatgcaggaagcactgatgtcgctgatgcagatggcgaaaacctctgcggcactggcaaaaatgcaggagcgcggcttgccgtacatctccgtgctgaccgacccgacgatgggcggtgtttctgcaagtttcgccatgctgggcgatctcaacatcgctgaaccgaaagcgttaatcggctttgccggtccgcgtgttatcgaacagaccgttcgcgaaaaactgccgcctggattccagcgcagtgaattcctgatcgagaaaggcgcgatcgacatgatcgtccgtcgtccggaaatgcgcctgaaactggcgagcattctggcgaagttgatgaatctgccagcgccgaatcctgaagcgccgcgtgaaggcgtagtggtacccccggtaccggatcaggaacctgaggcctgattaggaggttaatatgagtctgaatttccttgattttgaacagccgattgcagagctggaagcgaaaatcgattctctgactgcggttagccgtcaggatgagaaactggatattaacatcgatgaagaagtgcatcgtctgcgtgaaaaaagcgtagaactgacacgtaaaatcttcgccgatctcggtgcatggcagattgcgcaactggcacgccatccacagcgtccttataccctggattacgttcgcctggcatttgatgaatttgacgaactggctggcgaccgcgcgtatgcagacgataaagctatcgtcggtggtatcgcccgtctcgatggtcgtccggtgatgatcattggtcatcaaaaaggtcgtgaaaccaaagaaaaaattcgccgtaactttggtatgccagcgccagaaggttaccgcaaagcactgcgtctgatgcaaatggctgaacgctttaagatgcctatcatcacctttatcgacaccccgggggcttatcctggcgtgggcgcagaagagcgtggtcagtctgaagccattgcacgcaacctgcgtgaaatgtctcgcctcggcgtaccggtagtttgtacggttatcggtgaaggtggttctggcggtgcgctggcgattggcgtgggcgataaagtgaatatgctgcaatacagcacctattccgttatctcgccggaaggttgtgcgtccattctgtggaagagcgccgacaaagcgccgctggcggctgaagcgatgggtatcattgctccgcgtctgaaagaactgaaactgatcgactccatcatcccggaaccactgggtggtgctcaccgtaacccggaagcgatggcggcatcgttgaaagcgcaactgctggcggatctggccgatctcgacgtgttaagcactgaagatttaaaaaatcgtcgttatcagcgcctgatgagctacggttacgcgtaaaaaggagaatatatggatattcgtaagattaaaaaactgatcgagctggttgaagaatcaggcatctccgaactggaaatttctgaaggcgaagagtcagtacgcattagccgtgcagctcctgccgcaagtttccctgtgatgcaacaagcttacgctgcaccaatgatgcagcagccagctcaatctaacgcagccgctccggcgaccgttccttccatggaagcgccagcagcagcggaaatcagtggtcacatcgtacgttccccgatggttggtactttctaccgcaccccaagcccggacgcaaaagcgttcatcgaagtgggtcagaaagtcaacgtgggcgataccctgtgcatcgttgaagccatgaaaatgatgaaccagatcgaagcggacaaatccggtaccgtgaaagcaattctggtcgaaagtggacaaccggtagaatttgacgagccgctggtcgtcatcgagtaacgaggcgaacatgctggataaaattgttattgccaaccgcggcgagattgcattgcgtattcttcgtgcctgtaaagaactgggcatcaagactgtcgctgtgcactccagcgcggatcgcgatctaaaacacgtattactggcagatgaaacggtctgtattggccctgctccgtcagtaaaaagttatctgaacatcccggcaatcatcagcgccgctgaaatcaccggcgcagtagcaatccatccgggttacggcttcctctccgagaacgccaactttgccgagcaggttgaacgctccggctttatcttcattggcccgaaagcagaaaccattcgcctgatgggcgacaaagtatccgcaatcgcggcgatgaaaaaagcgggcgtcccttgcgtaccgggttctgacggcccgctgggcgacgatatggataaaaaccgtgccattgctaaacgcattggttatccggtgattatcaaagcctccggcggcggcggcggtcgcggtatgcgcgtagtgcgcggcgacgctgaactggcacaatccatctccatgacccgtgcggaagcgaaagctgctttcagcaacgatatggtttacatggagaaatacctggaaaatcctcgccacgtcgagattcaggtactggctgacggtcagggcaacgctatctatctggcggaacgtgactgctccatgcaacgccgccaccagaaagtggtcgaagaagcgccagcaccgggcattaccccggaactgcgtcgctacatcggcgaacgttgcgctaaagcgtgtgttgatatcggctatcgcggtgcaggtactttcgagttcctgttcgaaaacggcgagttctatttcatcgaaatgaacacccgtattcaggtagaacacccggttacagaaatgatcaccggcgttgacctgatcaaagaacagctgcgtatcgctgccggtcaaccgctgtcgatcaagcaagaagaagttcacgttcgcggccatgcggtggaatgtcgtatcaacgccgaagatccgaacaccttcctgccaagtccgggcaaaatcacccgtttccacgcacctggcggttttggcgtacgttgggagtctcatatctacgcgggctacaccgtaccgccgtactatgactcaatgatcggtaagctgatttgctacggtgaaaaccgtgacgtggcgattgcccgcatgaagaatgcgctgcaggagctgatcatcgacggtatcaaaaccaacgttgatctgcagatccgcatcatgaatgacgagaacttccagcatggtggcactaacatccactatctggagaaaaaactcggtcttcaggaaaaataagactgctaaagcgtcaaaaggccggattttccggccttttttattactggggatcgacaacccccataaggtacaatccccgctttcttcacccatcagggacgctcggtcgcctttcacattccgcgaaaattcataccgtcgagttacgcccgttctgcttgacctggtaaagttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacaggtgagctgataccgctcgccgcatgcacatgcagtcatgtcgtgc

SEQ ID NO: 35 - DNA, 8080 bp, Artificial Sequence
  Synthetic pNH243
accagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatttcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcacaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagccagtccgtttaggtgttttcacgagcaattgaccaacaaggacaggaggtattaatgtcggcgacgacgggcgcacgtagcgcctctgttggatgggcagaatcactgattgggttgcatttgggcaaggtcgccctgattacgggtggatctgccggcattggtgggcagattggtcgcttattggctttatctggtgcacgtgtgatgctggcggcacgtgatcgccacaaattggaacagatgcaagctatgattcagagtgaattagcggaagttggctacacagatgtggaggatcgcgttcatatcgcaccagggtgcgacgtttcaagtgaggcccaacttgcagacttggttgaacgcacattgtcagctttcggtaccgttgactatttaatcaataacgccggcattgcgggtgtagaggagatggttatcgacatgcccgtggagggttggcgtcatacgctgttcgcaaacctcatctcgaactacagccttatgcgtaagctggcaccactgatgaagaagcaggggtcggggtacatcctcaacgtaagctcgtatttcggaggggagaaagacgcagcaattccgtatcccaaccgtgccgactatgctgttagtaaagccggccagcgcgctatggctgaagtctttgcgcgctttttgggacccgagattcagatcaatgctattgcacctggacccgtagagggagatcgtctgcgcggaactggtgagcgtcctggattgttcgctcgccgtgcacgccttatcttggagaacaagcgtttaaatgagcttcatgctgcgttaatcgcggcagctcgtaccgatgaacgttcgatgcatgagttggttgaattgttgttgccgaatgacgtcgcggcgcttgaacaaaaccccgctgcccctaccgcacttcgtgagctggcgcgtcgcttccgcagtgagggagaccctgcagcgtcttcgtccagtgctttattaaatcgctccattgcggcgaaattacttgctcgtttgcacaatggtggatatgttctgccagcagacatttttgccaatttgccgaacccaccagacccttttttcacccgcgcccagatcgaccgcgaggctcgtaaggtacgcgatggaattatgggaatgctttatcttcagcgcatgcctacggaatttgatgttgcgatggcgacggtttattatttggcggaccgtgttgtctcaggcgagactttccatccgtctggaggtttgcgctacgaacgcaccccgacagggggagaattgtttggcctgccttcgccggaacgtttagcagagcttgtcggctccacagtctatctgatcggtgaacatttaactgagcatttaaacttgcttgcacgcgcttacttagagcgctacggggcacgccaggtagttatgatcgtagaaacggaaactggagctgaaaccatgcgtcgtttactgcacgaccacgtagaggcaggacgcttgatgacgattgtggctggtgaccagatcgaggccgcgattgaccaagcgattactcgttacggacgtccaggtcccgtagtctgtaccccttttcgtccattgcccactgttcctcttgtaggccgcaaggacagcgattggtctaccgtcttgagcgaggcagaattcgctgagttatgtgagcatcagttaacgcatcacttccgtgtagcacgttggatcgccctgtctgacggtgcacgtcttgccttagtcacgcccgagactacggcaactagcacaaccgagcagttcgccttggccaacttcattaagacaacgcttcatgctttcaccgcaacgatcggtgtagagtctgaacgcacagcgcagcgcatcctgatcaatcaagtcgatttaactcgtcgtgctcgcgcagaggagccgcgtgaccctcacgaacgtcaacaggaattggagcgcttcatcgaagccgtgctgttagttaccgcaccgttgccacccgaagcggacactcgttatgccggacgtatccaccgcggtcgcgcgattacggtataattaagaaaggaggtactcaatgtcaggaacagggcgcttggcgggaaaaattgccttgattacgggaggtgccggcaatattggctccgaattaacccgtcgcttcctggcagagggtgcgacagtcattatctctggacgtaatcgtgctaagctgactgcgctggccgagcgtatgcaagctgaggccggcgtccccgctaaacgcattgacttggaagttatggacggaagcgacccagttgccgtgcgcgcaggcattgaggctattgttgctcgccacggccagattgacattcttgtcaacaacgccggtagcgctggtgctcagcgtcgtttagcggaaatccctttaactgaagcagaattgggtcccggagcagaggaaacattgcacgctagtatcgccaatcttcttggaatgggctggcatctgatgcgcattgcggccccgcacatgcccgttggatcagctgttatcaatgtgtccaccatcttttctcgtgccgaatactacggacgtatcccatatgtgacccccaaagccgcactgaatgcgttaagccagctggctgcgcgcgaactgggcgcgcgtggaattcgcgtaaatacaatctttccgggtcccatcgaatcggaccgcattcgcactgtatttcaacgtatggatcagctgaagggccgtcctgagggtgacacggcgcaccatttcttgaacacgatgcgcctgtgtcgcgcaaacgaccaaggggcattagaacgccgcttcccttccgtcggcgatgtcgccgatgccgcggtcttcttggcttctgctgagtctgcagcattatctggggaaacgattgaggtgacccatggaatggagctgccggcctgttcggaaacgagtctgttagcgcgcactgatctgcgcactatcgatgcctctggccgtaccacccttatctgtgccggggatcagattgaggaggtaatggctttaacgggtatgctgcgcacatgcggatcggaggtaattattggttttcgtagcgccgccgcattagcacagtttgaacaggcagtaaatgagtcgcgtcgtttagctggcgcagactttaccccgccgatcgccttacctcttgatccgcgtgaccctgctacgatcgacgctgtattcgattggggagctggagaaaacactgggggtattcatgcggcggttatcctgcccgcgacatcccatgagcccgcaccgtgtgtaattgaagtagatgacgaacgtgtgctgaactttcttgcggacgaaatcaccgggacgattgtcattgctagccgtcttgctcgctactggcagtcacaacgcctgacgcctggggcgcgcgcccgcggaccacgtgtgatctttctgtctaatggggcagatcagaacggaaatgtttatggccgcattcagtcggcagccatcggtcaattaattcgtgtttggcgccacgaagccgagctggactatcaacgcgcgtctgccgcgggcgaccatgttctgccgcccgtatgggcaaaccaaatcgtacgttttgcaaaccgctcgctggagggtttggagttcgcctgcgcatggaccgctcaacttttacactcccaacgccatattaacgaaatcaccctgaacatcccagccaatatctaaatgacttaggagcgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttcggtccgttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgtttcccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttccttaggactgagcgtcaaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatataaggtgcactgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgcagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgaaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtc

SEQ ID NO: 36 - DNA, 13237 bp, Artificial Sequence
  Synthetic pNH265
ttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggctggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctcccaatgtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttttgacggctagctcagtcctagggataatgctagcaccagcctcgagggaaaccacgtaagctccggcgtttaaacacccataacagatacggactttctcaaaggagagttatcagtgaaaatccgcccgttacatgaccgtgtcatcatcaaacgcttggaagaagagcgtacctcggcgggcgggattgtcattccagatagcgcagctgaaaaaccgatgcgtggtgaaatcctggcagtgggcaatggaaaagtgcttgataatggagaggtacgtgctttacaggtgaaagtgggtgataaagtgctctttgggaaatacgcgggtacggaggttaaagtagatggggaagatgttgttgtcatgcgtgaagatgacattctggctgtgttagaatcttaatccgcgcacgacactgaacatacgaatttaaggaataaagataatggcgaaagaagttgtgtatcgtggtagtgcgcgccagcgtatgatgcagggtattgaaattctcgctcgcgccgctattccaacgctgggggcaaccggcccgagcgtcatgattcaacatcgcgccgatggtctgccacccatttctacacgcgatggcgttaccgtagcgaattctattgttttaaaagaccgtgtcgcgaacctgggtgcccgcctgctgcgcgacgtagccggtacaatgagccgtgaagccggcgacggcacgacgactgcgatcgtattggcccgccacatcgcccgtgagatgtttaaatcgctggccgtgggtgcagatccgatcgcgctgaaacgtggtatcgatcgcgccgttgctcgtgtgtccgaagatattggggcgcgtgcgtggcgtggcgataaagaaagcgtgatcctgggtgtcgctgctgtggcgacgaaaggcgaaccgggcgttggccgtctgctgctggaggctctcgatgcagtgggtgttcacggtgccgtttctatcgaactgggccaacgtcgtgaagatctgctggacgtcgtcgatggctatcgctgggaaaaaggttatttatctccctactttgtcacggaccgtgcccgcgaactcgcggaactggaggatgtctacctgctcatgaccgaccgcgaagtggttgacttcatcgaccttgtacctctgctggaggccgtgacggaagcaggaggctccctgctgattgccgcggatcgtgtgcacgaaaaggccttagcggggctgcttctgaatcacgtgcgcggtgtcttcaaggccgtggccgtaaccgctccgggttttggcgacaaacgcccgaaccgtttacttgacctggccgcgttaaccggcggtcgtgccgtgctcgaagctcaaggcgaccgtctggaccgtgttaccctcgcggatctgggccgtgtgcgccgtgccgtggtgtcggcagatgataccgcgctgcttggcatcccgggcaccgaagctagccgtgcacgcctcgaaggtctgcgtttagaagcagagcagtaccgtgcgctgaaaccagggcagggttctgccaccgggcgcctgcacgaacttgaagaaattgaagcgcgcattgtgggtctgtccggaaagagcgccgtttatcgcgtcggaggtgtgaccgatgtggaaatgaaagagcgcatggttcgcatcgaaaacgcttaccgttcggtggtaagtgcgctggaggaaggcgtgctccctggcggtggtgtcggctttctgggtagtatgccggtgcttgcggaattggaggcccgcgacgcagatgaagctcgcgggattgggattgtacgcagcgccttaacggagcctcttcgtattatcggcgaaaatagtggcttgagcggtgaagccgttgttgccaaagtcatggatcatgccaacccgggatggggttacgaccaggagtctggctctttttgcgacctgcatgcgcgtgggatctgggatgctgctaaagtgttacgtctcgcgttggagaaggcagcctctgttgctgggacctttctgacaaccgaagctgttgttctcgaaattccggatacagatgcgttcgcagggttcagtgcagaatgggctgccgccacgcgcgaagatccgcgcgtatgagtttaaacgcggccgcaatttgaacgcacccataacagatacggactttctcaaaggagagttatcaatgaatattcgtccattgcatgatcgcgtgatcgtcaagcgtaaagaagttgaaactaaatctgctggcggcatcgttctgaccggctctgcagcggctaaatccacccgcggcgaagtgctggctgtcggcaatggccgtatccttgaaaatggcgaagtgaagccgctggatgtgaaagttggcgacatcgttattttcaacgatggctacggtgtgaaatctgagaagatcgacaatgaagaagtgttgatcatgtccgaaagcgacattctggcaattgttgaagcgtaatccgcgcacgacactgaacatacgaatttaaggaataaagataatggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccatgctctgactctaaagcgattgctcaggttggtaccatctccgctaactccgacgaaaccgtaggtaaactgatcgctgaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgccaaagcaggcaaaccgctgctgatcatcgctgaagatgtagaaggcgaagcgctggcaactctggttgttaacaccatgcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtgggtgctgctaccgaagttgaaatgaaagagaaaaaagcacgcgttgaagatgccctgcacgcgacccgtgctgcggtagaagaaggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgaatgcatggttaccgacctgccgaaaaacgatgcagctgacttaggcgctgctggcggtatgggcggcatgatgtaagtttaaacgcggccgcaatttgaacgccagcacatggactctcgagtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggtgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctggacaatgtctccatacccgtttttttgggcgacctcgtcggaggttgtatgtccggtgttccgtgacgtcatcgggcattcatcattcatagaatgtgttacggaggaaacaagtaatggcacttagcaccgcaaccaaggccgcgacggacgcgctggctgccaatcgggcacccaccagcgtgaatgcacaggaagtgcaccgttggctccagagcttcaactgggatttcaagaacaaccggaccaagtacgccaccaagtacaagatggcgaacgagaccaaggaacagttcaagctgatcgccaaggaatatgcgcgcatggaggcagtcaaggacgaaaggcagttcggtagcctgcaggatgcgctgacccgcctcaacgccggtgttcgcgttcatccgaagtggaacgagaccatgaaagtggtttcgaacttcctggaagtgggcgaatacaacgccatcgccgctaccgggatgctgtgggattccgcccaggcggcggaacagaagaacggctatctggcccaggtgttggatgaaatccgccacacccaccagtgtgcctacgtcaactactacttcgcgaagaacggccaggacccggccggtcacaacgatgctcgccgcacccgtaccatcggtccgctgtggaagggcatgaagcgcgtgttttccgacggcttcatttccggcgacgccgtggaatgctccctcaacctgcagctggtgggtgaggcctgcttcaccaatccgctgatcgtcgcagtgaccgaatgggctgccgccaacggcgatgaaatcaccccgacggtgttcctgtcgatcgagaccgacgaactgcgccacatggccaacggttaccagaccgtcgtttccatcgccaacgatccggcttccgccaagtatctcaacacggacctgaacaacgccttctggacccagcagaagtacttcacgccggtgttgggcatgctgttcgagtatggctccaagttcaaggtcgagccgtgggtcaagacgtggaaccgctgggtgtacgaggactggggcggcatctggatcggccgtctgggcaagtacggggtggagtcgccgcgcagcctcaaggacgccaagcaggacgcttactgggctcaccacgacctgtatctgctggcttatgcgctgtggccgaccggcttcttccgtctggcgctgccggatcaggaagaaatggagtggttcgaggccaactaccccggctggtacgaccactacggcaagatctacgaggaatggcgcgcccgcggttgcgaggatccgtcctcgggcttcatcccgctgatgtggttcatcgaaaacaaccatcccatctacatcgatcgcgtgtcgcaagtgccgttctgcccgagcttggccaagggcgccagcaccctgcgcgtgcacgagtacaacggccagatgcacaccttcagcgaccagtggggcgagcgcatgtggctggccgagccggagcgctacgagtgccagaacatcttcgaacagtacgaaggacgcgaactgtcggaagtgatcgccgaactgcacgggctgcgcagtgatggcaagaccctgatcgcccagccgcatgtccgtggcgacaagctgtggacgttggacgatatcaaacgcctgaactgcgtcttcaagaacccggtgaaggcattcaattgaaacgggtgtcgggctccgtcacagggcggggcccgacgcacgatcgttcgatcaacctcaaaccaaaaaggaacatcgatatgagcatgttaggagaaagacgccgcggtctgaccgatccggaaatggcggccgtcattttgaaggcgcttcctgaagctccgctggacggcaacaacaagatgggttatttcgtcaccccccgctggaaacgcttgacggaatatgaagccctgaccgtttatgcgcagcccaacgccgactggatcgccggcggcctggactggggcgactggacccagaaattccacggcggccgcccttcctggggcaacgagaccacggagctgcgcaccgtcgactggttcaagcaccgtgacccgctccgccgttggcatgcgccgtacgtcaaggacaaggccgaggaatggcgctacaccgaccgcttcctgcagggttactccgccgacggtcagatccgggcgatgaacccgacctggcgggacgagttcatcaaccggtattggggcgccttcctgttcaacgaatacggattgttcaacgctcattcgcagggcgcccgggaggcgctgtcggacgtaacccgcgtcagcctggctttctggggcttcgacaagatcgacatcgcccagatgatccaactcgaacggggtttcctcgccaagatcgtacccggtttcgacgagtccacagcggtgccgaaggccgaatggacgaacggggaggtctacaagagcgcccgtctggccgtggaagggctgtggcaggaggtgttcgactggaacgagagcgctttctcggtgcacgccgtctatgacgcgctgttcggtcagttcgtccgccgcgagttctttcagcggctggctccccgcttcggcgacaatctgacgccattcttcatcaaccaggcccagacatacttccagatcgccaagcagggcgtacaggatctgtattacaactgtctgggtgacgatccggagttcagcgattacaaccgtaccgtgatgcgcaactggaccggcaagtggctggagcccacgatcgccgctctgcgcgacttcatggggctgtttgcgaagctgccggcgggcaccactgacaaggaagaaatcaccgcgtccctgtaccgggtggtcgacgactggatcgaggactacgccagcaggatcgacttcaaggcggaccgcgatcagatcgttaaagcggttctggcaggattgaaataatagaggaactattacgatgagcgtaaacagcaacgcatacgacgccggcatcatgggcctgaaaggcaaggacttcgccgatcagttctttgccgacgaaaaccaagtggtccatgaaagcgacacggtcgttctggtcctcaagaagtcggacgagatcaatacctttatcgaggagatccttctgacggactacaagaagaacgtcaatccgacggtaaacgtggaagaccgcgcgggttactggtggatcaaggccaacggcaagatcgaggtcgattgcgacgagatttccgagctgttggggcggcagttcaacgtctacgacttcctcgtcgacgtttcctccaccatcggccgggcctataccctgggcaacaagttcaccattaccagtgagctgatgggcctggaccgcaagctcgaagactatcacgcttaaggagaatgacatggcgaaactgggtatacacagcaacgacacccgcgacgcctgggtgaacaagatcgcgcagctcaacaccctggaaaaagcggccgagatgctgaagcagttccggatggaccacaccacgccgttccgcaacagctacgaactggacaacgactacctctggatcgaggccaagctcgaagagaaggtcgccgtcctcaaggcacgcgccttcaacgaggtggacttccgtcataagaccgctttcggcgaggatgccaagtccgttctggacggcaccgtcgcgaagatgaacgcggccaaggacaagtgggaggcggagaagatccatatcggtttccgccaggcctacaagccgccgatcatgccggtgaactatttcctggacggcgagcgtcagttggggacccggctgatggaactgcgcaacctcaactactacgacacgccgctggaagaactgcgcaaacagcgcggtgtgcgggtggtgcatctgcagtcgccgcactgaagggaggaagtctcgccctggacgcgacggcatcgccgtgaagtccagggggcagggatgccgttccgggccggcaggctggcccggaatctctggttttcagggggcgtgccggtccacggctcccccctccatctttcgtaaggaaatcaccatggtcgaatcggcatttcagccattttcgggcgacgcagacgaatggttcgaggaaccacggccccaggccggtttcttcccttccgcggactggcatctgctcaaacgggacgagacctacgcagcctatgccaaggatctcgatttcatgtggcggtgggtcatcgtccgggaagaaaggatcgtccaggagggttgctcgatcagcctggagtcgtcgatccgcgccgtgacgcacgtactgaattattttggtatgaccgaacaacgcgccccggcagaggaccggaccggcggagttcaacattgaacaggtaagtttatgcagcgagttcacactatcacggcggtgacggaggatggcgaatcgctccgcttcgaatgccgttcggacgaggacgtcatcaccgccgccctgcgccagaacatctttctgatgtcgtcctgccgggagggcggctgtgcgacctgcaaggccttgtgcagcgaaggggactacgacctcaagggctgcagcgttcaggcgctgccgccggaagaggaggaggaagggttggtgttgttgtgccggacctacccgaagaccgacctggaaatcgaactgccctatacccattgccgcatcagttttggtgaggtcggcagtttcgaggcggaggtcgtcggcctcaactgggtttcgagcaacaccgtccagtttcttttgcagaagcggcccgacgagtgcggcaaccgtggcgtgaaattcgaacccggtcagttcatggacctgaccatccccggcaccgatgtctcccgctcctactcgccggcgaaccttcctaatcccgaaggccgcctggagttcctgatccgcgtgttaccggagggacggttttcggactacctgcgcaatgacgcgcgtgtcggacaggtcctctcggtcaaagggccactgggcgtgttcggtctcaaggagcggggcatggcgccgcgctatttcgtggccggcggcaccgggttggcgccggtggtctcgatggtgcggcagatgcaggagtggaccgcgccgaacgagacccgcatctatttcggtgtgaacaccgagccggaattgttctacatcgacgagctcaaatccctggaacgatcgatgcgcaatctcaccgtgaaggcctgtgtctggcacccgagcggggactgggaaggcgagcagggctcgcccatcgatgcgttgcgggaagacctggagtcctccgacgccaacccggacatttatttgtgcggtccgccgggcatgatcgatgccgcctgcgagctggtacgcagccgcggtatccccggcgaacaggtcttcttcgaaaaattcctgccgtccggggcggcctgaaccggggaagtaccgtgaccaccgagcagttcccgccccaattcctgcgtgaaatgatcgagcagctggacgccagcatccaggagctcgcacgcaaggaaaagggacttgcggcatccctgggcacgggccgggtcgccgagctcaaggaatactgggaccacgttgttacaaccaattaaccaattctgactatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcg

SEQ ID NO: 37 - DNA, 6570 bp, Artificial Sequence
  Synthetic pLC130
ggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttcgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctttacatcgcatttttaataatttggatgacttcttctaacttaggtttacgaggatttgttaatgcacatgcatctttcatcgcattcttagctaaagtctcaatgtcttcttctttagcacctagttctttaaagccttttggaatgttaaggtctttagccattctttcgatcgctttaatagctttttcagctgcatcgtacgtacttagaccgtcgacattttcaccaagaaaagcagcgatttctgcataacgttccactttagaaattaagttaaatcgacatacatatggcagaaggaccgcattgcaaacgccatgagggaagttgtagaatcctcctaattggtgtgcaatcgcatgaacatagcctaaacccgcgttattgaatgccatgccagctaatgattgagcgaaggccatttgttcacgtgcttcaatgtcttttccatttgcaactgcacgcggcaagtatttagaaatgattttgatcgcctgaattgcaagtgcatctgtaattggagtagcaccagttgaaacatatgcttcaattgcatgagttaatgcatctaatccagtagcagcagttaaggacggaggcattccaaccattagctctgggtcgttgattgaaagtgtaggtgttacatgtttatccacaatggccattttcactttgcgttcagtatctgtgatgattgtgaatttagttaattcactgcctgtaccagctgttgtattaatcgcaattagcgggaccattggttcttttgatacatcgacaccttcataatcgtgaatttttccaccattagcagctactaatgcaatggcttttccggcatcatgtgaacttccgccgcccagagtgacaatgctgtcacagttttcagcgttatacgcttctaaaccttctgcgacgtttttatcggttggatttggttcggcttttggaaaaatggatacttccacaccagctgcacgaataatactggaaattttttcagaaagacctaaaccgtgaagaccagcatctgtaactaataaagcttttttcacaccaagatcagctaatcgagttccaacctcattaactgatcctgcaccaaatagattgactgaaggcataaaaaatgcactttgagtgtttgtcattaatatcctccttattgtaacctctgaagaaaccggcaacttactccagattcgcatggcgaccatacatcgttttggtatccaggcctttcttttccataaaacgcagaataaccgcgtcatagaagagcagcagcgtttgttcaaataagctacccatcggctgaattgtctcacgcgcttcagatttatctttcgggctacccggcatcttgatgacgatgtcagcgagctgcccaatcgtgctttcggggttgatggtcacggctgcaatcgttcctccgatactcttggctttctgggccatgctcaccaggcttttggtttcgccagaaccgctaccaataatcaaaatgtcctctttttcgtagttgggcgtcacagtttctccaaccacgtatgcatcgattcccatgtgcatcatacgcatcgcgaaactctttgccatgaagccagagcggccagcgccagcaacgaaaacttttttcgactgcaggatcccgttcaccagcgcttctgcttcttcatccgcaatctggtttacactgctgttcagttcctttacaatttccgccaaaaactctgtagtcaacatactaatcattattatcctcctatatcctataacggtacagcttcaggctagttacagcccttgcttaaccagtttgttaatcttttcggccgctgccttcttgtccgtttgatttgcgatcccgccgcctacaatgaccaaatccggttcagctttgataacctctggcagggtttcgagcttaatgccgcccgcgatggccgttttggcatttttcaccacggccttgatgcgtttcaggtcatccaacgggtttttccccaccgcttgaagatcgtaacccgcgtgcacacaaatataatccacgcccatttcgtcgacctgtttcgcgcgttcctccaggtttttcaccgcgatcatgtctactaagatcttcttgcccagtttttttgcttcttcaaccgcacctttaatggaaacatcctccgctgcagctaaaatggtcacaatatccgcaccgtgttccgccgctttagcaacttcgtacgccgccgcatccatcgtcttcatatcggccagaacctgcagatgcggaaaggcgtccttcaccgctttcacggcctgcaggccccagatcttaatcaccggtgtaccaatctcgacaatatccacatactcctgcacttcggccacgacctgttttgcttcttcgatgttaactaagtctaacgctaactgaagttccattatattcctcctttatggccctcgcgagtacagttatgcccaaaaaaacgggtatggagaaacagtagagagttgcgataaaaagcgtcaggtaggatccgctaatcttatggataaaaatgctatggcatagcaaagtgtgacgccgtgcaaataatcaatgtggacttttctgccgtgattatagacacttttgttacgcgtttttgtcatggctttggtcccgctttgttacagaatgcttttaataagcggggttaccggtttggttagcgagaagagccagtaaaagacgcagtgacggcaatgtctgatgcaatatggacaattggtttcttctctgaatggcgggagtatgaaaagtatggctgaagcgcaaaatgatcccctgctgccgggatactcgtttaatgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtttgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacggggttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgtatcagccaggcgaagctgcttttgagcaccacccggatgcctatcgccaccgtcggtcgcaatgttggttttgacgatcaactctatttctcgcgggtatttaaaaaatgcaccggggccagcccgagcgagttccgtgccggttgtgaagaaaaagtgaatgatgtagccgtcaagttgtcataattggtaacgaatcagacaattgacggcttgacggagtagcatagggtttgcagaatccctgcttcgtccatttgacaggcacattatgcatgccgcttcgccttcgcgcgcgaattgatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagagtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttcc

SEQ ID NO: 38 - DNA, 6450 bp, Artificial Sequence
  Synthetic pLC158
tctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggcataactgtactcgcgagggccataaaggaggaatataatggaacttcagttagcgttagacttagttaacatcgaagaagcaaaacaggtcgtggccgaagtgcaggagtatgtggatattgtcgagattggtacaccggtgattaagatctggggcctgcaggccgtgaaagcggtgaaggacgcctttccgcatctgcaggttctggccgatatgaagacgatggatgcggcggcgtacgaagttgctaaagcggcggaacacggtgcggatattgtgaccattttagctgcagcggaggatgtttccattaaaggtgcggttgaagaagcaaaaaaactgggcaagaagatcttagtagacatgatcgcggtgaaaaacctggaggaacgcgcgaaacaggtcgacgaaatgggcgtggattatatttgtgtgcacgcgggttacgatcttcaagcggtggggaaaaacccgttggatgacctgaaacgcatcaaggccgtggtgaaaaatgccaaaacggccatcgcgggcggcattaagctcgaaaccctgccagaggttatcaaagctgaaccggatttggtcattgtaggcggcgggatcgcaaatcaaacggacaagaaggcagcggccgaaaagattaacaaactggttaagcaagggctgtaactagcctgaagctgtaccgttataggatataggaggataataatgattagtatgttgactacagagtttttggcggaaattgtaaaggaactgaacagcagtgtaaaccagattgcggatgaagaagcagaagcgctggtgaacgggatcctgcagtcgaaaaaagttttcgttgctggcgctggccgctctggcttcatggcaaagagtttcgcgatgcgtatgatgcacatgggaatcgatgcatacgtggttggagaaactgtgacgcccaactacgaaaaagaggacattttgattattggtagcggttctggcgaaaccaaaagcctggtgagcatggcccagaaagccaagagtatcggaggaacgattgcagccgtgaccatcaaccccgaaagcacgattgggcagctcgctgacatcgtcatcaagatgccgggtagcccgaaagataaatctgaagcgcgtgagacaattcagccgatgggtagcttatttgaacaaacgctgctgctcttctatgacgcggttattctgcgttttatggaaaagaaaggcctggataccaaaacgatgtatggtcgccatgcgaatctggagtaagttgccggtttcttcagaggttacaataaggaggatattaatgaccactgctgcaccccaagaatttactgctgctgttgttgaaaaattcggtcatgacgtgaccgtgaaggatattgaccttccaaagccagggccacaccaggcattggtgaaggtactcacctccggcatctgccacaccgacctccacgccttggagggcgattggccagtaaagccggaaccaccattcgtaccaggacacgaaggtgtaggtgaagttgttgagctcggaccaggtgaacacgatgtgaaggtcggcgatattgtcggcaatgcgtggctctggtcagcgtgtggcacctgcgaatactgcatcaccggcagggaaactcagtgcaacgaagctgagtatggtggctacacccaaaatggatccttcggccagtacatgctggtggatacccgttacgccgctcgcatcccagacggcgtggactacctcgaagcagcaccaattctgtgtgcaggcgtgactgtctacaaggcactcaaagtctctgaaacccgcccgggccaattcatggtgatctccggtgtcggcggacttggccacatcgcagtccaatacgcagcggcgatgggcatgcgtgtcattgcggtagatattgccgatgacaagctggaacttgcccgtaagcacggtgcggaatttaccgtgaatgcgcgtaatgaagattcaggcgaagctgtacagaagtacaccaacggtggcgcacacggcgtgcttgtgactgcagttcacgaggcagcattcggccaggcactggatatggctcgacgtgcaggaacaattgtgttcaacggtctgccaccgggagagttcccagcatccgtgttcaacatcgtattcaagggcctgaccatccgtggatccctcgtgggaacccgccaagacttggccgaagcgctcgatttctttgcacgcggactaatcaagccaaccgtgagtgagtgctccctcgatgaggtcaatggtgtgcttgaccgcatgcgaaacggcaagattgatggtcgtgtggcaattcgctactaaagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacgaagttagctcactcattaggcaccgggatctcgaccgatgcccttgagagccttcaacccagtcagctccttccggtgggcgcggggcatgactaacatgagaattacaacttatatcgtatggggctgacttcaggtgctacatttgaagagataaattgcactgaaatctagagcggttcagtagaaaagatcaaaggatcttcttgagatcctttttttctgcgcgtaatcttttgccctgtaaacgaaaaaaccacctggggaggtggtttgatcgaaggttaagtcagttggggaactgcttaaccgtggtaactggctttcgcagagcacagcaaccaaatctgtccttccagtgtagccggactttggcgcacacttcaagagcaaccgcgtgtttagctaaacaaatcctctgcgaactcccagttaccaatggctgctgccagtggcgttttaccgtgcttttccgggttggactcaagtgaacagttaccggataaggcgcagcagtcgggctgaacggggagttcttgcttacagcccagcttggagcgaacgacctacaccgagccgagataccagtgtgtgagctatgagaaagcgccacacttcccgtaagggagaaaggcggaacaggtatccggtaaacggcagggtcggaacaggagagcgcaagagggagcgacccgccggaaacggtggggatctttaagtcctgtcgggtttcgcccgtactgtcagattcatggttgagcctcacggctcccacagatgcaccggaaaagcgtctgtttatgtgaactctggcaggagggcggagcctatggaaaaacgccaccggcgcggccctgctgttttgcctcacatgttagtcccctgcttatccacggaatctgtgggtaactttgtatgtgtccgcagcgcccgccgcagtctcacgcccggagcgtagcgaccgagtgagctagctatttgtttatttttctaaatacattcaaatatgtatccgctcatgagacaataaccctgataaatgcttcaataatattgaaaaaggaagagtatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgaggggccgcaagatccggccacgatgacccggtcgtcggttcagggcagggtcgttaaatagccgcttatgtctattgctggtttaccggtttattgactaccggaagcagtgtgaccgtgtgcttctcaaatgcctgaggtttcagcaaaaaacccctcaagacccgtttagaggccccaaggggttatgctagttattgctcagcggtggcagcagcctaggttaattaagctgcgctagtagac

SEQ ID NO: 39 - DNA, 16545 bp, Artificial Sequence
  Synthetic pBZ27
taatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctagattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttgcagccgaacgacctagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggcgacctcgtcggaggttgtatgtccggtgttccgtgacgtcatcgggcattcatcattcatagaatgtgttacggaggaaacaagtaatgacaaacactcaaagtgcattttttatgccttcagtcaatctatttggtgcaggatcagttaatgaggttggaactcgattagctgatcttggtgtgaaaaaagctttattagttacagatgctggtcttcacggtttaggtctttctgaaaaaatttccagtattattcgtgcagctggtgtggaagtatccatttttccaaaagccgaaccaaatccaaccgataaaaacgtcgcagaaggtttagaagcgtataacgctgaaaactgtgacagcattgtcactctgggcggcggaagttcacatgatgccggaaaagccattgcattagtagctgctaatggtggaaaaattcacgattatgaaggtgtcgatgtatcaaaagaaccaatggtcccgctaattgcgattaatacaacagctggtacaggcagtgaattaactaaattcacaatcatcacagatactgaacgcaaagtgaaaatggccattgtggataaacatgtaacacctacactttcaatcaacgacccagagctaatggttggaatgcctccgtccttaactgctgctactggattagatgcattaactcatgcaattgaagcatatgtttcaactggtgctactccaattacagatgcacttgcaattcaggcgatcaaaatcatttctaaatacttgccgcgtgcagttgcaaatggaaaagacattgaagcacgtgaacaaatggccttcgctcaatcattagctggcatggcattcaataacgcgggtttaggctatgttcatgcgattgcacaccaattaggaggattctacaacttccctcatggcgtttgcaatgcggtccttctgccatatgtatgtcgatttaacttaatttctaaagtggaacgttatgcagaaatcgctgcttttcttggtgaaaatgtcgacggtctaagtacgtacgatgcagctgaaaaagctattaaagcgatcgaaagaatggctaaagaccttaacattccaaaaggctttaaagaactaggtgctaaagaagaagacattgagactttagctaagaatgcgatgaaagatgcatgtgcattaacaaatcctcgtaaacctaagttagaagaagtcatccaaattattaaaaatgcgatgtaaaaaccaaaaaggaacatcgatatgacaacaaactttttcattccaccagccagcgtaattggacgcggtgcagtaaaggaagtaggaacaagacttaagcaaattggagctaagaaagcgcttatcgttacagatgcattccttcacagcacaggtttatctgaagaagttgctaaaaacattcgtgaagctggcgttgatgttgcgattttcccaaaagctcaaccagatccagcagatacacaagttcatgaaggtgtagatgtattcaaacaagaaaactgtgattcacttgtttctatcggtggaggtagctctcacgatacagctaaagcaatcggtttagttgcagcaaacggcggaagaatcaatgactatcaaggtgtaaacagcgtagaaaaaccagtcgttccagtagttgcaatcactacaacagctggtactggtagtgaaacaacatctcttgcggttattacagactctgcacgtaaagtaaaaatgcctgttattgatgagaaaattactccaactgtagcaattgttgacccagaattaatggtgaaaaaaccagctggattaacaatcgcaactggtatggatgcattgtcccatgcaattgaagcatatgttgcaaaaggtgctacaccagttactgatgcatttgctattcaagcaatgaaacttatcaatgaatacttaccaaaagcggttgcgaacggagaagacatcgaagcacgtgaaaaaatggcttatgcacaatacatggcaggagtggcatttaacaacggtggtttaggactagttcactctatttctcaccaagtaggtggagtttacaaattacaacacggaatctgtaactcagttaatatgccacacgtttgcgcattcaacctaattgctaaaactgagcgcttcgcacacattgctgagcttttaggtgagaatgttgctggcttaagcactgcagcagctgctgagagagcaattgtagctcttgaaagaatcaacaaatccttcggtatcccatctggctatgcagaaatgggcgtgaaagaagaggatatcgaattattagcgaaaaacgcatacgaagacgtatgtactcaaagcaacccacgcgttcctactgttcaagacattgcacaaatcatcaaaaacgctatgcatcatcaccatcaccactgatagaggaactattacgggagaatgacatggaacttcaattagctctagatttggtaaacattgaagaagcaaaacaagtagtagctgaggttcaggagtatgtcgatatcgtagaaatcggtactccggttattaaaatttggggtcttcaagctgtaaaagcagttaaagacgcattccctcatttacaagttttagctgacatgaaaactatggatgctgcagcatatgaagttgcgaaagcagctgagcatggcgctgatatcgtaacaattcttgcagcagctgaagatgtatcaattaaaggtgctgtagaagaagcgaaaaaacttggcaaaaaaatccttgttgacatgatcgcagttaaaaatttagaagagcgtgcaaaacaagtggatgaaatgggcgtagactacatttgcgtgcacgctggatacgatcttcaagcagtaggtaaaaacccattagatgatcttaagagaattaaagctgtcgtgaaaaatgcaaaaactgctattgcgggcggaatcaaattagaaacattacctgaagttatcaaagcagaaccggatcttgtcattgttggcggcggtattgctaaccaaactgataaaaaagcagcagctgaaaaaattaataaattagttaaacaagggttatgatcagcatgctgacaactgaatttttagctgaaattgtaaaagaattaaatagttcggttaaccaaatcgccgatgaagaagccgaagcactggttaacggaatccttcaatcaaagaaagtttttgtagccggtgcaggaagatccggttttatggctaaatccttcgcaatgcgaatgatgcacatgggtattgatgcctatgtcgttggcgaaaccgtaacacctaactatgaaaaagaagacatcttaatcattggatccggctcaggagaaacaaaaagtctcgtttccatggctcaaaaagcaaaaagcattggcggaaccatcgcggctgtaacgatcaaccctgaatcaacaattgggcaattagcggatatcgttattaaaatgccaggttcgcctaaagataaatcagaagctagagaaaccatccaaccaatgggatctctttttgaacaaaccttattattgttctatgatgctgtcattttgagattcatggagaaaaagggcttggatacaaaaacaatgtacggaagacatgctaatcttgagtagtccatctttcgtaaggaaatcaccatgatcaagattgcaccttctattctttcagctaattttgcacgacttgaagaagaaataaaagatgttgaacggggcggagccgattacattcatgttgatgtcatggatggtcattttgtgccaaatataacaattggcccattaattgtcgaggcaattagacctgtcacaaacttacctttagatgttcatttaatgatagaaaatccagatcaatacattgggacgtttgccaaagcaggtgctgatatattatctgtccatgttgaagcttgtactcatttgcacagaaccattcaatatattaaatctgaaggtataaaagctggagtggtattaaaccctcatactcccgtttcaatgattgaacatgtaatagaggatgttgatcttgtattgcttatgacggttaatcctggctttgggggacaatcattcattcattctgtcctacctaaaataaaacaagttgctaacatcgtaaaagagaaaaatttgcaggttgaaattgaagtagacggtggagtaaatcctgaaacggctaaactttgcgtagaagcaggagccaatgtccttgttgcaggttcagccatatataatcaagaggatagaagtcaagccattgcaaaaattagaaattgaacaggtaagtttccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggccttttttgacggctagctcagtcctagggataatgctagcaccagcctcgagggaaaccacgtaagctccggcgtttaaacacccataacagatacggactttctcaaaggagagttatcaatgagggaattgaaaagcgaaaagcgtgttcagtcgttagctatggaatttctctctgtagcacagcaagcagctctcgcttcttatccttggataggaaaaggtaataaaaacgaagttgatagggctggtacggaagctatgcgcaatcgactgaacctcattgatatgagcggtttaattgttattggtgaaggggaaatggacgaagctcctatgctttatattggagaggaactcggaacaggaaaaggaccccaactcgatattgcagtagaccctgttgatggaacgggtttaatggcaaaaggaatggataattcaatagcagtaattgctgcatccactagaggaagtttactgcatgccccagatatgtacatggaaaagatagctgtgggaccaaaagcaaaaggctgcgtaaatctagacgcatctttaacagaaaatatgaaatcagttgctaaagctttagggaaagatttaagagaattaactgtaatgatacaggatagaccacgtcatgatcatttgatccaacaagtaagagatgtaggggctagactcaaattattttctgatggtgacgttacaagggcaataggtactgcactcgaagaagtagacgttgatatattagtaggaactggcggtgctccagaaggagtaattgctgcaaccgcactgaagtgtttggggggagatttccaaggaagacttgctcctcaaaacgaagaagaatttgatcgctgtattacgatgggaataacagatccaagaaaaattttcacaatagatgaaattgtaaaatcagatgattgcttttttgtagcaacaggaataactgacggactgcttataaatggtattcgaaaaaaagaagatggtttaatgcaaacgcactcttttcttacaattggaggaagcagcgtaaaataccaatttattgaagcttatcattgataataaacgtaataaatgacgtttgatgtatctaattgaatgctcttttatgttgatgtttcggaactgtttcggaaccctcctttttcggttaatattctcaaaattcagttttatgtcgcagtaacgattagcaacttcaattagatataacgaagaaagcgatttcccgatcttatcatgttagtttcctcagcttgaaactttcctgattatccgtaaaggaaatacacttgtaaagcagatgttaaaggaaaaatttccctttgttaagttgtgaacaagatggtatctcatccttgtccatctctgaatggcaataaattattcttgtgtgacagtgtgaaaaccttcgtttcagagattcattttcatgaaagaacataggtggtaagaaatccccaaaatgatcctaagaccataatctagggatgtcacaaaaagtcaacccctatgataggatggttcagatattagacagcttagctctccactatctgaacgatcctttatgaagttatcaaagagcaataaataaacggaaaattacctcgaaagaggattctaaaaatactatataaggagtgggaattatgccattagtttcaatgaaggatatgttaaatcatggaaaagaaaatggatatgctgttggacagtttaacatcaataatcttgagtttggtcaagcgattttacaagctgcagaggaagagaagtctcctgttattatcggggtatctgtaggtgctgctaattacatgggtggatttaagttaattgttgatatggtcaaatcattaatggattcatataacgtaacggtaccagttgctattcatcttgaccatggtccaagtcttgagaaatgtgtacaagccatccatgctggatttacatctgttatgatcgatggttcccatcttccacttgaagaaaatattgaattaacaaaacgtgtggttgaaatagcacattctgttggcgtatctgttgaggcagagctaggtcgtatcggtggacaagaagatgatgtagtagctgaatcattttatgctatcccttcagaatgtgagcaattagttcgtgaaacaggagtagactgctttgcacctgcgttaggttctgtccatggtccgtataaaggtgaaccaaaacttggttttgatcggatggaggaaattatgaaattaacaggtgttcctcttgttctccacggtggtacaggtattccaacaaaagatattcaaaaagctatttcgcttggtacagcaaaaattaacgtaaatacagaaagccaaattgctgctacaaaagccgttcgagaagttttaaataacgatgctaagctgtttgatcctcgcaaatttttagcaccggctcgggaagcgattaaagaaaccattaaaggtaaaatgcgtgaatttggatcttcaggtaaagcttaataaaaaacagacattatgggaggggaaatcgtgctccaacaaaaaatagatattgatcagttatccattcaaactattagaactctatcaattgatgcaattgaaaaggttggatcaggccatccggggatgccaatgggggctgccccgatggcctatacactttggacaaaatttatgaattacaatccaagcaacccgaattggtttaatcgtgaccgttttgtattgtcagcgggacacggatccatgttattatacagcctattacatttaactggttatgatctatcattagaagatttgaaaaacttccgccaatggggaagcaaaacacctggtcaccctgaatttggccatacacctggggttgatgccacaacaggtccgttagggcaaggtattgccatggcagttgggatggcgatggctgaaagacatttagcgtctaaatacaatcgttataaatttaatattattgatcactacacatacagcatttgtggcgatggggacttgatggaaggtgtatctgcagaggcagcttcacttgcagggcaccttaaacttggtcgcttaattgtattatacgattcaaatgatatttctcttgatggcgatcttcatatgtcatttagtgagagtgttcaagatcgttttaaagcatacggctggcaagtacttcgtgttgaggacggcaatgatatcgattcaatcgcaaaagcgatagctgaagcgaaaaacaacgaagaccaaccaacattaattgaagtcaaaacaataattggatacggctcaccgaataaaggtggaaagtctgatgcgcacggctcaccacttggaaaagaggaaataaagcttgtaaaagaacattacaactggaaatatgatgaggatttttatatccctgaagaagtaaaagaatattttagagaattaaaagaagcagcagagaagaaggaacaagcatggaatgagttgttcgcacaatataaagaagcatatccagcacttgcaaaggaattagaacaagcgattaatggtgaactaccagaaggctgggatgctgatgttcctgtttaccgtgtcggagaagataaacttgctactcgttcttccagtggtgcagtgttaaatgctctagcgaaaaatgttccgcaactacttggcggttctgcggatttagcttcatctaataaaacgctactaaaaggggaagcaaatttcagcgctacagattatagcggacgtaatatttggtttggtgttcgtgaatttggaatgggtgctgctgtcaacggaatggccctacacggtggtgtaaaagtatttggagcaacattctttgtattctctgattatttacgtccggccattcgtctctcagcattaatgaaactaccagttatttatgtctttacacatgatagcgttgctgtaggtgaagatggaccaacacatgaaccaattgaacaattggcatccttacgtgcaatgcctggtatctctacaattcgcccggctgatggcaatgagacagctgcagcttggaagttggcgttagaaagtaaagacgaaccaacagctcttatcctctcacgtcaagacttaccaacacttgttgattctgaaaaagcgtatgagggtgttaaaaaaggtgcatatgtgatctctgaagcaaaaggtgaagttgctggtttgttattagcatctggttctgaagttgctttagctgttgaagcacaagcagcgctggaaaaggaaggtatttatgtttcagttgttagtatgcctagctgggatcgttttgaaaaacaatctgatgcatacaaagaaagtgtacttccaaaaaacgtaaaagcacgtcttggtattgaaatgggggcttccttaggttggagtaaatatgttggtgataacggtaacgtcctcgccattgatcaatttggatcctcagcaccaggagataaaataattgaagaatacggttttacagtcgaaaatgtcgtttctcattttaaaaagcttctctaaaagtcttgcccttgtttaatcggctgttttggcactggagatcttgtacaggaatagttcataatttctgaaagcaagctccgatagtgtttagcatttttttgaataaatccacagaagagttcaagacggcactttcctctcaaacgaaatcaaagcaaacggtacaatgtgcgcaacttttgatgtaggaaaatggcctgtgccaattctgaattggtctctcaccaattttttagccgttcttattcttctgtgaaatcctcacaaagtgttttacaaacaacttcctatcttgttttaaagcagcgagtgactgttctttactgaacttcacctgaacctacttgcgaacctcttccaacgctttgatgaacgattgaatcatcatggaactcatcaatgatcactaacacaatgcggtaaggcggtttcaatcgctgttttgtatgctttccacatattgatcacaattcgatgaggcgttcaggatgagatattgtttcaagatctcaataacgtcctctttttttttctgtttttgttctgtttttctttttcaacttctttttctcaaacttgaaaaagtaaacaaacaagagattattatcagaaaattcgttcatttatagaagatatggaggaaaacaagaatgaataagattgcagtattaactagcggcggggatgcaccaggaatgaacgctgctattcgtgcggtcgttcgaagaggaatctttaaaggactagatgtttatggtgtaaaaaatggctacaaaggtttaatgaatgggaattttgtttcaatgaacctcggaagtgtgggtgatattattcaccgaggaggcactatcttacaaactacacgctgtaaagagtttaagacagctgaagggcaacaacaggctttagcacagctaaaaaaagaaggcattgatggcttaatcgtgattggtggagatggcacttttgaaggtgcgagaaaattaactgcccaagagtttccaactattggtattccggcaaccattgacaatgacattgcagggacggaatatacaattggatttgatactgctgtgaacacagcagtggaagcaattgataaaattcgtgatacggcagcctctcatgatcgtatctatgtcgttgaagtaatgggccgcaatgcaggagacatcgctctatgggcaggaatgtgtgcgggagcagaatcaattattatcccagaagccgaccatgatgtggaagatgtaattgatcgtattaaacaaggatatcagcgaggaaaaacgcacagtattattgtggttgcagaaggggcatttaatggagtaggagcaatagaaattggtagagcaattaaagagaaaacaggatttgacacaaaggtaaccatacttgggcatattcaacgtgggggatctcctagcgcttacgaccgaatgatgagcagtcagatgggtgcaaaagccgtggatttgctggttgaaggcaaaaaaggtctgatggtaggattaaaaaatggtcaactgattcatacaccttttgaggaagctgcgaaagataagcatacggttgatttgtccatctaccatttagcaagaagtctttctttatagaccggggaagtaccgtgaccaccgagcagttcccgccccaattcctgcgtgaaatgatcgagcagctggacgccagcatccaggagctcgcacgcaaggaaaagggacttgcggcatccctgggcacgggccgggtcgccgagctcaaggaatactgggaccacgttgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacaggtgagctgataccgctcgccgcatgcacatgcagtcatgtcgtgc

(19) United States
(12) Patent Application Publication
(10) Pub. No.: US 2020/0048639 A1
(43) Pub. Date: Feb. 13, 2020

(54) CULTURE MODIFIED TO CONVERT METHANE OR METHANOL TO 3-HYDROXYPROPRIONATE

(71) Applicant: Industrial Microbes, Inc., Emeryville, CA (US)

(72) Inventors: Elizabeth Jane CLARKE, San Francisco, CA (US); Derek Lorin GREENFIELD, Kensington, CA (US); Noah Charles HELMAN, El Cerrito, CA (US); Stephanie Rhianon JONES, Berkeley, CA (US); Baolong ZHU, San Diego, CA (US)

(21) Appl. No.: 16/486,459

(22) PCT Filed: Feb. 17, 2018
(86) PCT No.: PCT/IB2018/050978

§ 371 Date: Aug. 15, 2019

Related U.S. Application Data
(60) Provisional application No. 62/460,565, filed on Feb. 17, 2017.
(60) Provisional application No. 62/530,671, filed on Jul. 10, 2017.
(60) Provisional application No. 62/578,709, filed on Oct. 30, 2017.

(51) Int. Cl. C12N 15/52; C12P 7/42; C12N 9/02

(52) U.S. Cl. CPC C12N 15/52; C12P 7/42; C12N 9/0008; C12Y 604/01002; C12Y 101/01244; C12Y 503/01027; C12Y 114/13025; C12Y 102/01075; C12Y 101/02007; C12Y 401/02043

(57) ABSTRACT

Provided are engineered organisms which can convert methane or methanol to 3-hydroxypropionate.

### FIELD

Provided are methods and compositions for the conversion of methane and/or methanol into 3-hydroxypropionate in an engineered microorganism.

### BACKGROUND

Biological enzymes are catalysts capable of facilitating chemical reactions, often at ambient temperature and/or pressure. Some chemical reactions are catalyzed by either inorganic catalysts or certain enzymes, while others can be catalyzed by just one of these. For industrial uses, enzymes are advantageous catalysts if the alternative process requires expensive or energy-intensive conditions, such as high temperature or pressure, or if the complete process is to be integrated with other enzyme-catalyzed steps. Enzymes can also be engineered to control the range of raw materials or substrates required and/or the range of products formed.

Recent technological advances in synthetic biology have demonstrated the power and versatility of enzymatic pathways in living cells to convert organic molecules into industrial products. The petrochemical processes that currently manufacture industrial products may be replaced by biotechnological processes that can often provide the same products at a lower cost and with a lower environmental impact. The discovery of new pathways and enzymes that can operate and be engineered in genetically tractable microorganisms will further advance synthetic biology.

Sugar (including simple sugars, disaccharides, starches, carbohydrates, cellulosic sugars, and sugar alcohols) is often a raw material for biological fermentations. But sugar has a relatively high cost as a raw material which severely limits the economic viability of the fermentation process. Although synthetic biology could expand to produce thousands of products that are currently petroleum-sourced, companies often must limit themselves to the production of select niche chemicals due to the high cost of sugar.

One-carbon compounds, such as methane and methanol, are significantly less expensive raw materials compared to sugar. Given the enormous supply of natural gas and the emergence of renewable methane-production technologies, methane is expected to remain inexpensive for decades to come. Accordingly, industrial products made by engineered microorganisms from methane or its derivatives, such as methanol, will be less expensive to manufacture than those made by sugar and should remain so for decades.

3-hydroxyproprionate (and 3-hydroxypropionic acid) is one of the top value-added platform compounds among renewable biomass products. Currently, 3-hydroxyproprionate (3HP) is gaining increased interest because of its versatile applications. For instance, 3-hydroxyproprionate can be easily converted to a range of bulk chemicals, such as acrylic acid, ethyl acrylate, butyl acrylate, other acrylic acid esters, 1,3-propanediol (1,3-PD), 3-hydroxypropionaldehyde (3-HPA), and malonic acid. These bulk chemicals find applications in high-performance plastics, water-soluble paints, coatings, fibers, adhesives, chemicals for industrial water treatment, and super-absorbent polymers for diapers. In addition, 3-hydroxyproprionate and its derivatives can be polymerized to form higher-value materials.

### BRIEF DESCRIPTION

Provided are methods for converting methane or methanol into 3-hydroxypropionate in an engineered microorganism.

Some embodiments provide a synthetic culture comprising one or more microorganisms comprising one or more modifications that improve the production of a product from a substrate, wherein the substrate comprises methane and/or methanol. In some embodiments, the substrate comprises methane. In some embodiments, the substrate comprises methanol. In some embodiments, the product comprises 3-hydroxyproprionate. In some embodiments, the product comprises a substance derived from acetyl-CoA and/or malonyl-CoA.

In some embodiments, the one or more microorganisms comprises Escherichia coli. In some embodiments, the one or more microorganisms comprises a first at least one microorganism and a second at least one microorganism, wherein the first at least one microorganism produces methanol from methane and the second at least one microorganism produces 3-hydroxypropionate from methanol.

In some embodiments, the one or more modifications comprise exogenous polynucleotides and/or deletion of one or more genes. In some embodiments, the exogenous polynucleotides encode one or more polypeptides comprising exogenous polynucleotides encoding polypeptides selected from one or more polypeptides comprising methane monooxygenase (EC 1.14.13.25), malonyl-CoA reductase (EC 1.2.1.75), acetyl-CoA carboxylase (EC 6.4.1.2), methanol dehydrogenase (EC 1.1.1.244 or EC 1.1.2.7), 3-hexulose-6-phosphate synthase (EC 4.1.2.43), and/or 6-phospho-3-hexuloisomerase (EC 5.3.1.27). In some embodiments, the methanol dehydrogenase comprises a methanol dehydrogenase from Bacillus methanolicus, Bacillus stearothermophilus, and/or Corynebacterium glutamicum. In some embodiments, the methane monooxygenase comprises the soluble methane monooxygenase from Methylococcus capsulatus (Bath). In some embodiments, the acetyl-CoA carboxylase comprises accABCD from Escherichia coli. In some embodiments, the malonyl-CoA reductase comprises a malonyl-CoA reductase from Chloroflexus aurantiacus.

In some embodiments, the malonyl-CoA reductase has one or more substitutions. In some embodiments, the one or more substitutions comprise A763T, V793A, L818P, L843Q, N940S, N940V, T979A, K1106R, K1106W, and/or S1114R.

In some embodiments, the one or more modifications comprise at least one exogenous polynucleotide comprising one or more of rpeP, glpXP, fbaP, tktP, and/or pfkP genes from Bacillus methanolicus. In some embodiments, the one or more modifications comprise deletion of glpK, gshA, frmA, pgi, gnd, and/or lrp.

In some embodiments, the exogenous polynucleotides comprise one more of more of a nucleic acid comprising a sequence comprising one or more of SEQ ID NOs: 34-39. In some embodiments, the exogenous polynucleotides comprise one or more of a coding region comprising the nucleotide sequence of the coding region of the plasmids set forth in one or more of SEQ ID NOs: 34-39. In some embodiments, the one or more polypeptides comprise polypeptides having one or more amino acid sequences comprising one or more sequences set forth in any one or more of SEQ ID NOs: 1-33. In some embodiments, the one or more polypeptides comprise one or more substitutions. In some embodiments, the one or more substitutions comprise conservative substitutions. In some embodiments, the one or more polypeptides comprise polypeptides having an amino acid sequence comprising one or more sequences that are about 95% identical to one or more of the sequence set forth in SEQ ID NOs: 1-33.

Some aspects provide a method for producing a product, comprising culturing any of the synthetic cultures provided herein under suitable culture conditions and for a sufficient period of time to produce the product.

### BRIEF DESCRIPTION OF THE DRAWINGS

FIG. 1 depicts results for six (6) experiments wherein a co-culture was split into two vials after which the headspace was injected with either unlabeled or ¹³C-methane. The top panel shows the fraction of 3HP that is singly-¹³C-labeled. The middle panel shows the fraction of 3HP that is doubly-¹³C-labeled. The bottom panel shows the fraction of 3HP that is triply-¹³C-labeled.

### DETAILED DESCRIPTION

### A. Definitions

The disclosure provides microorganisms engineered to functionally produce 3-hydroxyproprionate from methane or methanol. Compositions and methods comprising using said microorganisms to produce chemicals are further provided. The methods provide for superior low-cost production as compared to existing sugar-consuming fermentation.

As used herein, “amino acid” shall mean those organic compounds containing amine (—NH₂) and carboxyl (—COOH) functional groups, along with a side chain (R group) specific to each amino acid. The key elements of an amino acid are carbon (C), hydrogen (H), oxygen (O), and nitrogen (N), although other elements are found in the side chains of certain amino acids.

As used herein, “conservative amino acid substitution” refers to a substitution in which an amino acid residue is substituted by another amino acid residue having a side chain (R group) with similar chemical properties (e.g., charge or hydrophobicity). In general, a conservative amino acid substitution should not substantially change the functional properties of a protein. The following six groups each contain amino acids that are often, depending upon context, considered conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

As used herein, the term “culturing” is intended to mean the growth or maintenance of a microorganism under laboratory or industrial conditions. The culturing of microorganisms is a standard practice in the field of microbiology. Microorganisms can be cultured using liquid or solid media as a source of nutrients for the microorganisms. In addition, some microorganisms can be cultured in defined media, in which the liquid or solid media are generated by preparation using purified chemical components. The composition of the culture media can be adjusted to suit the microorganism or the industrial purpose for the culture.

As used herein, the term “dehydrogenase” is intended to mean an enzyme belonging to the group of oxidoreductases that oxidizes a substrate by a reduction reaction that removes one or more hydrogen atoms from a substrate to an electron acceptor. Methanol dehydrogenases are dehydrogenase enzymes which catalyze the conversion of methanol into formaldehyde.

As used herein, the term “endogenous polynucleotides” is intended to mean polynucleotides derived from naturally occurring polynucleotides in a given organism. The term “endogenous” refers to a referenced molecule or activity that is present in the host. Similarly, the term when used in reference to expression of an encoding nucleic acid or polynucleotide refers to expression of the encoding nucleic acid or polynucleotide contained within the microbial organism.

As used herein, the term “enzyme” or “enzymatically” shall refer to biological catalysts. Enzymes accelerate, or catalyze, chemical reactions. Like all catalysts, enzymes increase the rate of reaction by lowering the activation energy.

As used herein, “exogenous” is intended to mean something, such as a gene or polynucleotide that originates outside of the organism of concern or study. An exogenous polynucleotide, for example, may be introduced into an organism by introduction into the organism of an encoding nucleic acid, such as, for example, by integration into a host chromosome or by introduction of a plasmid. When used in reference to a biosynthetic activity, the term refers to an activity that is introduced into a reference organism, such as a microorganism or synthetic culture as set forth in the invention. As an example, exogenous expression of an encoding nucleic acid can utilize either or both a heterologous or homologous encoding nucleic acid. A nucleic acid need not include all of its relevant or even complete coding regions on a single polymer and the invention provided herein contemplates having complete or partial coding sequences on different polymers.

As used herein, the term “exogenous polynucleotides” is intended to mean polynucleotides that are not derived from naturally occurring polynucleotides in a given organism. Exogenous polynucleotides may be derived from polynucleotides present in a different organism. The exogenous polynucleotides can be introduced into the organism by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism.

The term “heterologous” refers to a molecule or activity derived from a source other than the referenced species whereas “homologous” refers to a molecule or activity derived from the host microbial organism. As set forth in the invention a nucleic acid need not include all of its relevant or even complete coding regions on a single polymer and the invention provided herein contemplates having complete or partial coding regions on different polymers.

As used herein, the term “enzyme specificity” or “specificity of an enzyme” is intended to mean the degree to which an enzyme is able to catalyze a chemical reaction on more than one substrate molecule. An enzyme that can catalyze a reaction on exactly one molecular substrate, but is unable to catalyze a reaction on any other substrate, is said to have very high specificity for its substrate. An enzyme that can catalyze chemical reactions on many substrates is said to have low specificity. In some cases, the specificity of an enzyme is described relative to one or more defined substrates.

As used herein, a “gene” is a sequence of DNA or RNA, which codes for a molecule that has a function. The DNA is first copied into RNA. The RNA can be directly functional or be the intermediate template for a protein that performs a function. Genes can acquire mutations in their sequence, leading to different variants, known as alleles, in the population.

As used herein, “modification,” “genetic alteration,” “genetically altered,” “genetic engineering,” “genetically engineered,” “genetic modification,” “genetically modified,” “genetic regulation,” or “genetically regulated” shall be used interchangeably and refer to direct or indirect manipulation of an organism's genome or genes to produce, for example, a desired effect, such as a desired phenotype. Genetic alteration includes a set of technologies that can be used to change genetic makeup, which ultimately could lead to the suppression or enhancement of phenotype or expression of a gene, as used herein. Genetic alteration shall also include the ability to reduce or prevent expression of a gene or genes. Genetic alteration techniques shall include, for example, but are not be limited to, molecular cloning, gene knockouts, gene targeting, mutation, homologous recombination, gene deletion, gene knockdown, gene silencing, gene addition, genome editing, gene attenuation, or any technique that may be used to suppress or alter the expression of a gene and a phenotype as known to one skilled in the art.

As used herein, “gene deletion” or “deletion” refers to a mutation or genetic modification in which a sequence of DNA is lost, deleted, or modified. A gene may be deleted to alter an organism's genome or to produce a desired effect or desired phenotype. Gene deletion may be used, for example, without limitation, as a method to suppress, alter, or enhance a particular phenotype.

As used herein, the term “gene knockdown” refers to a technique by which expression of one or more genes are reduced. Reduction can occur by any method known to one skilled in the art such as genetic modification, CRISPR interference, or by treatment with a reagent such as a short DNA or RNA oligonucleotide that has a sequence complimentary to either a gene or an mRNA transcript.

As used herein, the term “gene knockout” refers to a procedure whereby a gene is made inoperative.

As used herein, “gene silencing,” “silencing,” or “silenced” refers to the regulation of a gene, in particular, without limitation, the down regulation of a gene. Specifically, the term refers to the ability to reduce or prevent the expression of a certain gene. Gene silencing can occur at any cellular process, such as, for example, without limitation, during transcription or translation. Any methods of gene silencing well known in the art may be used such as, for example, without limitation, RNA interference and the use of antisense oligonucleotides.

As used herein, the term “homology” or “homologous” refer to the degree of biological shared ancestry in the evolutionary history of life. Homology or homologous may also refer to sequence homology, the biological homology between protein or polynucleotide sequences with respect to shared ancestry as determined by the closeness of nucleotide or protein sequences. Homology among proteins or polynucleotides is typically inferred from their sequence similarity. Alignments of multiple sequences are used to indicate which regions of each sequence are homologous. The term “percent homology” often refers to “sequence similarity.” The percentage of identical residues (percent identity) or the percentage of residues conserved with similar physiochemical properties (percent similarity), e.g. leucine and isoleucine, is usually used to quantify homology. Partial homology can occur where a segment of the compared sequences has a shared origin.

As used herein, the term “improved production of a product from a substrate” is intended to mean a situation in which a microorganism or synthetic culture has been modified in some way, such as, for example, without limitation, through genetic modification, so that, under a set of conditions and relative to the original strain, the modified strain produces a product from the substrate or produces a product from the substrate faster than the rate from an unmodified microorganism or synthetic culture. A direct comparison of two strains can be made by growing the microorganisms or synthetic cultures under identical conditions and measuring the amount of product produced by each.

As used herein, the term “methane monooxygenase enzyme” is intended to mean the class of enzymes and enzyme complexes capable of oxidizing a carbon-hydrogen bond of the methane molecule to result in a molecule of methanol. Naturally occurring methane-consuming microorganisms have evolved at least two classes of methane monooxygenase enzymes: soluble and particulate. Any enzyme or enzyme complex of these categories, any mutated enzyme or complex, or any researcher-designed enzyme or enzyme complex that converts methane into methanol would be considered a methane monooxygenase enzyme. Many of these enzymes are known to also oxidize a wide range of substrates, such as methane to methanol or ethane into ethanol, and thus, are relevant for the purpose of this invention (see, for example, WO/2017/087731 and WO/2015/160848, each of which is incorporated by reference herein, including any drawings).

As used herein, the terms “microbe”, “microbial,” “microbial organism” or “microorganism” are intended to mean any organism that exists as a microscopic cell that is included within the domains of archaea, bacteria, or eukarya. Therefore, the term is intended to encompass prokaryotic or eukaryotic cells or organisms having a microscopic size and includes bacteria, archaea, and eubacteria of all species as well as eukaryotic microorganisms such as yeast and fungi. The term also includes cell cultures of any species that can be cultured for the production of a product.

As used herein, “naturally occurring” shall refer to microorganisms or cultures normally found in nature.

As used herein, an “operon” shall refer to a functioning unit of genomic DNA containing a cluster of genes under the control of a single promoter. The genes are transcribed together into an mRNA strand and either translated together in the cytoplasm or undergo trans-splicing to create monocistronic mRNAs that are translated separately, i.e. several strands of mRNA that each encode a single gene product. The result of this is that the genes contained in the operon are either expressed together or not at all. Several genes may be co-transcribed to define an operon.

The terms “polynucleotide,” “oligonucleotide,” “nucleotide sequence,” and “nucleic acid sequence” are intended to mean one or more polymers of nucleic acids and include, but are not limited to, coding regions, which are transcribed or translated into a polypeptide or chaperone, appropriate regulatory or control sequences, controlling sequences, e.g., translational start and stop codons, promoter sequences, ribosome binding sites, polyadenylation signals, transcription factor binding sites, termination sequences, regulatory domains and enhancers, among others. A polynucleotide, as used herein, need not include all of its relevant or even complete coding regions on a single polymer and the invention provided herein contemplates having complete or partial coding region on different polymers.

As used herein, a “peptide” refers to short chains of amino acid monomers linked by peptide (amide) bonds. Covalent chemical bonds are formed when the carboxyl group of one amino acid reacts with the amino group of another. The shortest peptides are dipeptides, consisting of 2 amino acids joined by a single peptide bond, followed by tripeptides, tetrapeptides, etc.

As used herein, a “polypeptide” or “protein” is a long, continuous, and unbranched peptide chain. Peptides are normally distinguished from polypeptides and proteins on the basis of size, and as an arbitrary benchmark can be understood to contain approximately 50 or fewer amino acids. Proteins consist of one or more polypeptides arranged in a biologically functional way, often bound to ligands such as coenzymes and cofactors, or to another protein or other macromolecule, such as, for example, DNA or RNA.

Amino acids that have been incorporated into peptides are termed “residues” due to the release of either a hydrogen ion from the amine end or a hydroxyl ion from the carboxyl end, or both, as a water molecule is released during formation of each amide bond. All peptides except cyclic peptides have an N-terminal and C-terminal residue at the end of the peptide.

As used herein, “product” shall refer to 3-hydroxyproprionate and 3-hydroxypropionic acid and related molecules and derivatives. Related molecules include, for example, without limitation, acrylic acid, ethyl acrylate, butyl acrylate, other acrylic acid esters, 1,3-propanediol (1,3-PD), 3-hydroxypropionaldehyde (3-HPA), and malonic acid. Related products also include polymerized forms of 3-hydroxyproprionate, polymerized forms of acrylic acid, and polymerized forms of acrylic acid derivatives. Related products further include substances that derived from acetyl-CoA and/or malonyl-CoA.

As used herein, “promoter” shall refer to a region of DNA that initiates transcription of a particular gene. Promoters are located near the transcription start sites of genes, on the same strand and upstream on the DNA (towards the 5′ region of the sense strand). Promoters can be about 30-1000 base pairs long.

As used herein, the term “substrate” shall refer to a chemical species being used in a chemical reaction. In some embodiments, the substrate is methane or methanol.

As used herein, “sufficient period of time” shall refer to a time period required to grow microorganisms or a synthetic culture to produce a product, such as, for example, a product of interest. In that sense, a sufficient period of time can be the amount of time that enables the microorganisms, or enables the synthetic culture of interest, to produce the product. For example, without limitation, an industrial scale culture may require as little as 5 minutes to begin production of detectable amounts of a product. Some synthetic cultures may be active for weeks.

As used herein, the term “suitable conditions” is intended to mean any set of culturing parameters that provide the microorganism with an environment that enables the culture to consume the available nutrients. In so doing, the microbiological culture may grow and/or produce products, chemicals, or by-products. Culturing parameters may include, but not be limited to, such features as the temperature of the culture media, the dissolved oxygen concentration, the dissolved carbon dioxide concentration, the rate of stirring of the liquid media, the pressure in the vessel, etc.

As used herein, the term “synthetic” is intended to mean a culture or microorganism, for example, without limitation, that has been manipulated into a form not normally found in nature. For example, a synthetic culture or microorganism shall include, without limitation, a culture or microorganism that has been manipulated to express a polypeptide that is not naturally expressed or transformed to include a synthetic polynucleotide of interest that is not normally included.

As used herein, the term “synthetic culture” is intended to mean at least one microorganism, or group of microorganisms, that has been manipulated into a form not normally found in nature.

### B. Methane or Methanol and 3-Hydroxyproprionate

3-hydroxyproprionate is one of the top value-added platform compounds among renewable biomass products. Currently, 3-hydroxyproprionate is gaining increased interest because of its versatile applications. 3-hydroxyproprionate can be easily converted to a range of products, such as acrylic acid, ethyl acrylate, butyl acrylate, other acrylic acid esters, 1,3-propanediol (1,3-PD), 3-hydroxypropionaldehyde (3-HPA), and malonic acid. In addition, 3-hydroxyproprionate can be polymerized to form materials.

In some embodiments, the substrate comprises methane. In some embodiments, the substrate comprises methanol. In some embodiments, the product comprises 3-hydroxyproprionate.

In some embodiments, the product further comprises acrylic acid, 1,3-propanediol (1,3-PD), 3-hydroxypropionaldehyde (3-HPA), and malonic acid. In some embodiments, the product comprises a polymerized form of 3-hydroxyproprionate. In some embodiments, the polymerized form of 3-hydroxyproprionate is biodegradable. In some embodiments, the product further comprises acrylic acid. In some embodiments, the product is a substance derived from acetyl-CoA and/or malonyl-CoA.

### C. Enzymes

In some embodiments, the one or more polypeptides comprise methane monooxygenase. The methane monooxygenase enzymes class are enzyme complexes capable of oxidizing a carbon-hydrogen bond of the methane molecule to result in a molecule of methanol. Naturally occurring methane-consuming microorganisms have evolved at least two classes of methane monooxygenase enzymes: soluble and particulate. Any enzyme or enzyme complex of these categories, any mutated enzyme or complex, or any researcher-designed enzyme or enzyme complex that converts methane into methanol would be considered a methane monooxygenase enzyme. Many of these enzymes are known to also oxidize a wide range of substrates, such as methane to methanol or ethane into ethanol, and thus, are relevant as embodiments of the invention.

In some embodiments, the one or more polypeptides comprise malonyl-CoA reductase. Malonyl CoA reductase (malonate semialdehyde-forming) (EC 1.2.1.75, NADP-dependent malonyl CoA reductase, malonyl CoA reductase (NADP)) is an enzyme with systematic name malonate semialdehyde:NADP⁺ oxidoreductase (malonate semialdehyde-forming). Malonyl-CoA reductase enzyme catalyzes the following chemical reaction malonate semialdehyde+CoA+NADP⁺↔malonyl-CoA+NADPH+H⁺. The enzyme may require Mg²⁺.

In some embodiments, the malonyl-CoA reductase comprises a malonyl-CoA reductase from Chloroflexus aurantiacus.

In some embodiments, the one or more polypeptides comprise acetyl-CoA carboxylase. Acetyl-CoA carboxylase (ACC) is an enzyme that catalyzes the irreversible carboxylation of acetyl-CoA to produce malonyl-CoA through its two catalytic activities, biotin carboxylase (BC) and carboxyltransferase (CT). ACC is a multi-subunit enzyme in most prokaryotes and in the chloroplasts of most plants and algae, whereas it is a large, multi-domain enzyme in the endoplasmic reticulum of most eukaryotes. The most important function of ACC is to provide the malonyl-CoA substrate for the biosynthesis of fatty acids. The activity of ACC can be controlled at the transcriptional level as well as by small molecule modulators and covalent modification. In some embodiments, the activity of the ACC is manipulated or controlled.

In some embodiments, the acetyl-CoA carboxylase comprises accABCD from Escherichia coli.

In some embodiments, the one or more polypeptides comprise methanol dehydrogenase (“MDH”). A methanol dehydrogenase (EC 1.1.1.244 or EC 1.1.2.7) is an enzyme that catalyzes the chemical reaction: methanol↔formaldehyde+2 electrons+2H⁺. How the electrons are captured and transported depends upon the kind of methanol dehydrogenase. A common electron acceptor in biological systems is nicotinamide adenine dinucleotide (NAD⁺) and some enzymes use a related molecule called nicotinamide adenine dinucleotide phosphate (NADP⁺). An NAD⁺-dependent methanol dehydrogenase (EC 1.1.1.244) was first reported in a Gram-positive methylotroph and is an enzyme that catalyzes the chemical reaction methanol+NAD⁺↔formaldehyde+NADH+H⁺. Thus, the two substrates of this enzyme are methanol and NAD⁺, whereas its 3 products are formaldehyde, NADH, and H⁺. This enzyme belongs to the family of oxidoreductases, specifically those acting on the CH—OH group with NAD⁺ or NADP⁺ as acceptor. The systematic name of this enzyme class is methanol:NAD⁺ oxidoreductase. This enzyme participates in methanol metabolism.

In some embodiments, the methanol dehydrogenase comprises a methanol dehydrogenase from Bacillus methanolicus and/or Corynebacterium glutamicum.

In some embodiments, the one or more polypeptides comprise 3-hexulose-6-phosphate synthase (“HPS”). 3-hexulose-6-phosphate synthase (EC 4.1.2.43, 3-hexulo-6-phosphate synthase, hexulophosphate synthase D-arabino-3-hexulose 6-phosphate formaldehyde-lyase, 3-hexulosephosphate synthase, 3-hexulose phosphate synthase, HPS) is an enzyme with systematic name D-arabino-hex-3-ulose-6-phosphate formaldehyde-lyase (D-ribulose-5-phosphate-forming). This enzyme catalyzes the reaction D-arabino-hex-3-ulose 6-phosphate↔D-ribulose 5-phosphate+formaldehyde. The enzyme may require Mg²⁺ or Mn²⁺ for maximal activity.

In some embodiments, the one or more polypeptides comprise 6-phospho-3-hexuloisomerase (“PHI”). 6-phospho-3-hexuloisomerase (EC 5.3.1.27, 3-hexulose-6-phosphate isomerase, hexulose-6-phosphate isomerase, phospho-3-hexuloisomerase, PHI, 6-phospho-3-hexulose isomerase, phospho-hexulose isomerase) is an enzyme with systematic name D-arabino-hex-3-ulose-6-phosphate isomerase. This enzyme catalyzes the reaction D-arabino-hex-3-ulose 6-phosphate↔D-fructose 6-phosphate. This enzyme plays a key role in the ribulose-monophosphate cycle of formaldehyde fixation.

### D. Methods

In some embodiments, provided herein is a microorganism or synthetic culture expressing one or more exogenous nucleic acids encoding one or more polypeptides and having a genetic modification or deletion of one or more genes native to the microorganism or synthetic culture. Some embodiments provide a synthetic culture comprising one or more microorganisms comprising one or more modifications that improve the production of a product from a substrate. In some embodiments, the one or more modifications comprise exogenous polynucleotides or deletion of one or more genes.

In some embodiments, the one or more modifications comprise at least one exogenous polynucleotide comprising one or more of rpeP, glpXP, fbaP, tktP, and/or pfkP genes from Bacillus methanolicus. In some embodiments, the one or more modifications comprise deletion of glpK, gshA, frmA, glpK, gnd, pgi, and/or lrp from Escherichia coli.

In some embodiments, the exogenous polynucleotides comprise one more of more of a nucleic acid comprising a sequence comprising one or more of SEQ ID NOs: 34-39. In some embodiments, the exogenous polynucleotides comprise one or more of a codon region comprising the nucleotide sequence of the coding region of the plasmids set forth in one or more of SEQ ID NOs: 34-39. In some embodiments, the one or more polypeptides comprise polypeptides having one or more amino acid sequences comprising one or more sequences set forth in any one or more of SEQ ID NOs: 1-33. In some embodiments, the one or more polypeptides comprise one or more substitutions. In some embodiments, the one or more substitutions comprise conservative substitutions. In some embodiments, the one or more polypeptides comprise polypeptides having an amino acid sequence comprising one or more sequences that are about 95% identical to one or more of the sequences set forth in SEQ ID NOs: 1-33.

Expression of one or more exogenous nucleic acids in a microorganism or synthetic culture can be accomplished by introducing into the microorganism or synthetic culture a nucleic acid comprising a nucleotide sequence encoding the one or more polypeptides under the control of regulatory elements that permit expression in the microorganism or synthetic culture.

Nucleic acids encoding the one or more polypeptides can be introduced into a microorganism or synthetic culture by any method known to one of skill in the art without limitation (see, for example, Hinnen et al. (1978) Proc. Natl. Acad. Sci. USA 75:1292-3; Cregg et al. (1985) Mol. Cell. Biol. 5:3376-3385; Goeddel et al. eds, 1990, Methods in Enzymology, vol. 185, Academic Press, Inc., CA; Krieger, 1990, Gene Transfer and Expression—A Laboratory Manual, Stockton Press, NY; Sambrook et al., 1989, Molecular Cloning—A Laboratory Manual, Cold Spring Harbor Laboratory, NY; and Ausubel et al., eds., Current Edition, Current Protocols in Molecular Biology, Greene Publishing Associates and Wiley Interscience, NY). Exemplary techniques include, but are not limited to, spheroplasting, electroporation, PEG 1000 mediated transformation, and lithium acetate- or lithium chloride-mediated transformation. In some embodiments, the nucleic acid is an extrachromosomal plasmid. In some embodiments, the nucleic acid is a chromosomal integration vector that can integrate the nucleotide sequence into the chromosome of the microorganism or synthetic culture.

Expression of genes may be modified. In some embodiments, expression of the one of more exogenous or endogenous nucleic acids is modified. For example, the copy number of an enzyme or one or more polypeptides in a microorganism or synthetic culture may be altered by modifying the transcription of the gene that encodes the enzyme or one or more polypeptides. This can be achieved, for example, by modifying the copy number of the nucleotide sequence encoding the enzyme or one or more polypeptides (e.g., by using a higher or lower copy number expression vector comprising the nucleotide sequence, or by introducing additional copies of the nucleotide sequence into the genome of the microorganism or synthetic culture, or by introducing additional nucleotide sequences into the genome of the microorganism or synthetic culture that express the same or similar polypeptide, or by genetically modifying or deleting or disrupting the nucleotide sequence in the genome of the microorganism or synthetic culture), by changing the order of coding sequences on a polycistronic mRNA of an operon, or by breaking up an operon into individual genes, each with its own control elements. The strength of the promoter, enhancer, or operator to which the nucleotide sequence is operably linked may also be manipulated or increased or decreased or different promoters, enhancers, or operators may be introduced.

Alternatively, or in addition, the copy number of one or more polypeptides may be altered by modifying the level of translation of an mRNA that encodes the enzyme or one or more polypeptides. This can be achieved, for example, by modifying the stability of the mRNA, modifying the sequence of the ribosome binding site, modifying the distance or sequence between the ribosome binding site and the start codon of the enzyme coding sequence, modifying the entire intercistronic region located upstream of or adjacent to the 5′ side of the start codon of the enzyme coding region, stabilizing the 3′-end of the mRNA transcript using hairpins and specialized sequences, modifying the codon usage of an enzyme, altering expression of rare codon tRNAs used in the biosynthesis of the enzyme, and/or increasing the stability of an enzyme, as, for example, via mutation of its coding sequence.

Expression of exogenous or endogenous nucleic acids may be modified or regulated by targeting particular genes. For example, without limitation, in some embodiments of the methods described herein, the microorganism or synthetic culture is contacted with one or more nucleases capable of cleaving, i.e., causing a break at a designated region within a selected site. In some embodiments, the break is a single-stranded break, that is, one but not both strands of the target site is cleaved. In some embodiments, the break is a double-stranded break. In some embodiments, a break-inducing agent is used. A break-inducing agent is any agent that recognizes and/or binds to a specific polynucleotide recognition sequence to produce a break at or near a recognition sequence. Examples of break-inducing agents include, but are not limited to, endonucleases, site-specific recombinases, transposases, topoisomerases, and zinc finger nucleases, and include modified derivatives, variants, and fragments thereof.

In some embodiments, a recognition sequence within a selected target site can be endogenous or exogenous to a microorganism or synthetic culture's genome. When the recognition site is an endogenous or exogenous sequence, it may be a recognition sequence recognized by a naturally occurring, or native break-inducing agent. Alternatively, an endogenous or exogenous recognition site could be recognized and/or bound by a modified or engineered break-inducing agent designed or selected to specifically recognize the endogenous or exogenous recognition sequence to produce a break. In some embodiments, the modified break-inducing agent is derived from a native, naturally occurring break-inducing agent. In other embodiments, the modified break-inducing agent is artificially created or synthesized. Methods for selecting such modified or engineered break-inducing agents are known in the art.

In some embodiments, the one or more nucleases is a CRISPR/Cas-derived RNA-guided endonuclease. CRISPR may be used to recognize, genetically modify, and/or silence genetic elements at the RNA or DNA level or to express heterologous or homologous genes. CRISPR may also be used to regulate endogenous or exogenous nucleic acids. Any CRISPR/Cas system known in the art finds use as a nuclease in the methods and compositions provided herein. CRISPR systems that find use in the methods and compositions provided herein also include those described in International Publication Numbers WO 2013/142578 A1, WO 2013/098244 A1 and Nucleic Acids Res (2017) 45 (1): 496-508, the contents of which are hereby incorporated in their entireties.

In some embodiments, the one or more nucleases is a TAL-effector DNA binding domain-nuclease fusion protein (TALEN). TAL effectors of plant pathogenic bacteria in the genus Xanthomonas play important roles in disease, or trigger defence, by binding host DNA and activating effector-specific host genes. see, e.g., Gu et al. (2005) Nature 435:1122-5; Yang et al., (2006) Proc. Natl. Acad. Sci. USA 103:10503-8; Kay et al., (2007) Science 318:648-51; Sugio et al., (2007) Proc. Natl. Acad. Sci. USA 104:10720-5; Romer et al., (2007) Science 318:645-8; Boch et al., (2009) Science 326(5959):1509-12; and Moscou and Bogdanove, (2009) 326(5959):1501, each of which is incorporated by reference in their entirety. A TAL effector comprises a DNA binding domain that interacts with DNA in a sequence-specific manner through one or more tandem repeat domains. The repeated sequence typically comprises 34 amino acids, and the repeats are typically 91-100% homologous with each other. Polymorphism of the repeats is usually located at positions 12 and 13, and there appears to be a one-to-one correspondence between the identity of repeat variable-diresidues at positions 12 and 13 with the identity of the contiguous nucleotides in the TAL-effector's target sequence.

The TAL-effector DNA binding domain may be engineered to bind to a desired sequence, and fused to a nuclease domain, e.g., from a type II restriction endonuclease, typically a nonspecific cleavage domain from a type II restriction endonuclease such as FokI (see e.g., Kim et al. (1996) Proc. Natl. Acad. Sci. USA 93:1156-1160). Other useful endonucleases may include, for example, Hhal, HindIII, Nod, BbvCI, EcoRI, BglI, and AlwI. Thus, in preferred embodiments, the TALEN comprises a TAL effector domain comprising a plurality of TAL effector repeat sequences that, in combination, bind to a specific nucleotide sequence in a target DNA sequence, such that the TALEN cleaves target DNA within or adjacent to the specific nucleotide sequence. TALENS useful for the methods provided herein include those described in WO10/079430 and U.S. Patent Application Publication No. 2011/0145940, which is incorporated by reference herein in its entirety.

In some embodiments, the one or more of the nucleases is a zinc-finger nuclease (ZFN). ZFNs are engineered break-inducing agents comprised of a zinc finger DNA binding domain and a break-inducing agent domain. Engineered ZFNs consist of two zinc finger arrays (ZFAs), each of which is fused to a single subunit of a non-specific endonuclease, such as the nuclease domain from the FokI enzyme, which becomes active upon dimerization.

Useful zinc-finger nucleases include those that are known and those that are engineered to have specificity for one or more sites. Zinc finger domains are amenable for designing polypeptides which specifically bind a selected polynucleotide recognition sequence. Thus, they are amenable to modifying or regulating expression by targeting particular genes.

In some embodiments, the activity of one or more genes native to the microorganism or synthetic culture is modified. The activity of one or more genes native to the microorganism or synthetic culture can be modified in a number of other ways, including, but not limited to, gene silencing or any other form of genetic modification, expressing a modified form of the polypeptides or one or more polypeptides that exhibits increased or decreased solubility in the microorganism or synthetic culture, expressing an altered form of the polypeptides or one or more polypeptides that lacks a domain through which the activity of the enzyme is inhibited, expressing a modified form of the polypeptides that has a higher or lower k_{cat} or a lower or higher Kₘ for a substrate, or expressing an altered form of the enzyme or one or more polypeptides or protein product of the one or more genes native to the microorganism or synthetic culture that is more or less affected by feed-back or feed-forward regulation by another molecule in the pathway.

In some embodiments, the enzymes or one or more polypeptides or one or more genes native to the microorganism or synthetic culture are modified. It will be recognized by one skilled in the art that absolute identity to the enzymes or one or more polypeptides or one or more genes native to the microorganism or synthetic culture is not strictly necessary. For example, changes in a particular gene or polynucleotide comprising a sequence encoding a polypeptide or an enzyme can be performed and screened for activity. Such modified or mutated polynucleotides and polypeptides can be screened for expression or function using methods known in the art.

Those of skill in the art will recognize that, due to the degenerate nature of the genetic code, a variety of polynucleotides differing in their nucleotide sequences can be used to encode one or more genes native to the microorganism or synthetic culture or a given enzyme or one or more polypeptides of the disclosure. Due to the inherent degeneracy of the genetic code, other polynucleotides, which encode substantially the same or functionally equivalent polypeptides, can also be used. The disclosure includes polynucleotides of any sequence that encode the amino acid sequences of the polypeptides and proteins of the enzymes or one or more polypeptides utilized in the methods of the disclosure.

In similar fashion, a polypeptide can typically tolerate one or more amino acid substitutions, deletions, and insertions in its amino acid sequence without loss or significant loss of a desired activity. The disclosure includes such one or more polypeptides with different amino acid sequences than the specific proteins described herein so long as the modified or variant polypeptides have an activity that is identical or similar to the referenced polypeptide. Accordingly, the amino acid sequences encoded by the DNA sequences shown herein merely illustrate embodiments of the disclosure.

The disclosure also includes one or more polypeptides with different amino acid sequences than the specific proteins described herein if the modified or variant polypeptides have an activity that is desirable yet different from referenced polypeptide. In some embodiments, an enzyme may be altered by modifying the gene that encodes the enzyme so that the expressed protein is more or less active than the wild type version. As an example, any of the expressed methane monooxygenases, malonyl-CoA reductases, acetyl-CoA carboxylase, methanol dehydrogenase (“MDH”), 3-hexulo-6-phosphate synthase, and/or 6-phospho-3-hexuloisomerase proteins may be more or less active according to substitutions.

As will be understood by those of skill in the art, it can be advantageous to modify a coding sequence to enhance expression in a particular host, such as, without limitation, Escherichia coli. The genetic code is redundant with 64 possible codons, but most organisms typically use a subset of these codons. Codons can be substituted, without any resultant change to the amino acid sequence of the corresponding protein, to increase or decrease the translation rate of the sequence, in a process sometimes called “codon optimization”.

Optimized coding sequences can be prepared, for example, to increase the rate of translation or to produce recombinant RNA transcripts having desirable properties, such as a longer half-life, as compared with transcripts produced from a non-optimized sequence. Translation stop codons can also be modified to reflect host preference.

In addition, homologs of enzymes or the one or more polypeptides or the proteins encoded by the one or more genes native to the microorganism or synthetic culture useful for the compositions and methods provided herein are encompassed by the disclosure. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

It is recognized that residue positions that are not identical often differ by conservative amino acid substitutions. In cases where two or more amino acid sequences differ from each other by conservative substitutions, the percent sequence identity or degree of homology may practically be adjusted upwards to correct for the conservative nature of the substitution. Means for making this adjustment are well known to those of skill in the art (See, e.g., Pearson W. R., 1994, Methods in Mol Biol 25: 365-89).

Sequence homology and sequence identity for polypeptides is typically measured using sequence analysis software. A typical algorithm used to compare a molecular sequence to a database containing a large number of sequences from different organisms is the computer program BLAST. When searching a database containing sequences from a large number of different organisms, it is typical to compare amino acid sequences.

Furthermore, any of the one or more genes native to the microorganism or synthetic culture or genes encoding the enzymes or one or more polypeptides or genes native to the microorganism or synthetic culture (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in yeast, bacteria, or any other suitable cell or organism.

For example, amino acid sequence variants of the protein(s) can be prepared by mutations in the DNA. Methods for mutagenesis and nucleotide sequence alterations include, for example, Kunkel, (1985) Proc Natl Acad Sci USA 82:488-92; Kunkel, et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff, et al., (1978) Atlas of Protein Sequence and Structure (Natl Biomed Res Found, Washington, D.C.).

Techniques known to those skilled in the art may be suitable to identify additional homologous genes and homologous enzymes. Generally, analogous genes and/or analogous enzymes can be identified by functional analysis and will have functional similarities. As an example, to identify homologous or analogous biosynthetic pathway genes, proteins, or enzymes, techniques may include, but are not limited to, cloning a gene by PCR using primers based on a published sequence of a gene/enzyme of interest or by degenerate PCR using degenerate primers designed to amplify a conserved region among a gene of interest.

Further, one skilled in the art can use techniques to identify homologous or analogous genes, proteins, or enzymes with functional homology or similarity. Techniques include examining a cell or cell culture for the catalytic activity of an enzyme through in vitro enzyme assays for the activity (e.g. as described herein or in Kiritani, K., Branched-Chain Amino Acids Methods Enzymology, 1970), then isolating the enzyme with the activity through purification, determining the protein sequence of the enzyme through techniques such as Edman degradation, design of PCR primers to the likely nucleic acid sequence, amplification of the DNA sequence through PCR, and cloning of the nucleic acid sequence. To identify homologous or similar genes and/or homologous or similar proteins, analogous genes and/or analogous proteins, techniques also include comparison of data concerning a candidate gene or enzyme with databases such as BRENDA, KEGG, or MetaCYC. The candidate gene or proteins may be identified within the above-mentioned databases in accordance with the teachings herein.

In some embodiments, the microorganism or synthetic culture expressing one or more polypeptides has one or more genes native to the microorganism or synthetic culture that have been genetically modified, deleted, or whose expression has been reduced or eliminated. In some embodiments, the araBAD genes have been deleted. In some embodiments, the frmA gene and/or the gshA gene has been deleted. In some embodiments, the pgi gene and/or the gnd gene has been deleted. In some embodiments, the glpK gene has been deleted. In some embodiments, the lrp gene has been deleted.

Reduction or elimination of expression may occur through any method known to one skilled in the art and all ways of genetically modifying, deleting, and/or of reducing or eliminating expression of genes native to the microorganism or synthetic culture are provided herein. In particular, one skilled in the art will understand that any form of genetic alteration or genetic engineering or genetic modification, such as those set forth above related to expression, may be used as an alternative to deletion. In some embodiments, other forms of genetic modification that may be used as an alternative to deletion include, for example, without limitation, gene knockouts, mutation, gene targeting, homologous recombination, gene knockdown, gene silencing, gene addition, molecular cloning, gene attenuation, genome editing, CRISPR intereference, or any technique that may be used to suppress or alter or enhance a particular phenotype.

In some embodiments, the one or more genes native to the microorganism or synthetic culture can be altered in other ways, including, but not limited to, expressing a modified form where the modified form exhibits increased or decreased solubility in the microorganism or synthetic culture, expressing an altered form that lacks a domain through which activity is inhibited, or expressing an altered form that is more or less affected by feed-back or feed-forward regulation by another molecule in a pathway expressed in the microorganism or synthetic culture. In some embodiments, the strength of the promoter, enhancer, or operator to which the nucleotide sequence for the one or more genes native to the microorganism or synthetic culture is operably linked may also be manipulated, decreased or increased or different promoters, enhancers, or operators may be introduced.

### E. Cells

Some embodiments disclose a synthetic culture. As used herein, the term “synthetic culture” is intended to mean at least one microorganism, or group of microorganisms, that has been manipulated into a form not normally found in nature.

Some embodiments include a microorganism that exists as a microscopic cell that is included within the domains of archaea, bacteria, or eukarya. In some embodiments, the microorganism is at least one of Escherichia coli, Bacillus subtilis, Bacillus methanolicus, Pseudomonas putida, Saccharomyces cerevisiae, Pichia pastoris, Pichia methanolica, Salmonella enterica, Corynebacterium glutamicum, Klebsiella oxytoca, Anaerobiospirillum succiniciproducens, Actinobacillus succinogenes, Mannheimia succiniciproducens, Rhizobium etli, Gluconobacter oxydans, Zymomonas mobilis, Lactococcus lactis, Lactobacillus plantarum, Streptomyces coelicolor, Clostridium acetobutylicum, Pseudomonas fluorescens, Schizosaccharomyces pombe, Kluyveromyces lactis, Kluyveromyces marxianus, Aspergillus terreus, Aspergillus niger, Yarrowia lipolytica, Hansenula polymorpha, Issatchenkia orientalis, Candida sonorensis, Candida methanosorbosa, and Candida utilis. In some embodiments, the microorganism is Escherichia coli.

In some embodiments, conversion of methane into methanol is catalyzed in one microorganism and conversion of methanol into 3-hydroxypropionate is catalyzed in a second, genetically distinct microorganism. In some embodiments, conversion of methane into methanol and conversion of methanol into 3-hydroxypropionate are both catalyzed in a single microorganism. In some embodiments, the single microorganism comprises the enzymes methane monooxygenase, methanol dehydrogenase, 3-hexulose-6-phosphate synthase, 6-phospho-3-hexuloisomerase, and malonyl-CoA reductase. In some embodiments, the single microorganism further comprises the enzyme acetyl-CoA carboxylase. In some embodiments, the single microorganism is Escherichia coli.

### F. Sequences

TABLE S
Sequences

| | | Region | |
|---|---|---|---|
| SEQ | | and/or | |
| ID | Mol- | Desig- | |
| NO | ecule | nation | Sequence |
| 1. | accA | pNH241 | MSLNFLDFEQPIAELEAKIDSLTAVSRQ |
| | | | DEKLDINIDEEVHRLREKSVELTRKIFA |
| | | | DLGAWQIAQLARHPQRPYTLDYVRLAFD |
| | | | EFDELAGDRAYADDKAIVGGIARLDGRP |
| | | | VMIIGHQKGRETKEKIRRNFGMPAPEGY |
| | | | RKALRLMQMAERFKMPIITFIDTPGAYP |
| | | | GVGAEERGQSEAIARNLREMSRLGVPVV |
| | | | CTVIGEGGSGGALAIGVGDKVNMLQYST |
| | | | YSVISPEGCASILWKSADKAPLAAEAMG |
| | | | IIAPRLKELKLIDSIIPEPLGGAHRNPE |
| | | | AMAASLKAQLLADLADLDVLSTEDLKNR |
| | | | RYQRLMSYGYA* |
| 2. | accB | pNH241 | MDIRKIKKLIELVEESGISELEISEGEE |
| | | | SVRISRAAPAASFPVMQQAYAAPMMQQP |
| | | | AQSNAAAPATVPSMEAPAAAEISGHIVR |
| | | | SPMVGTFYRTPSPDAKAFIEVGQKVNVG |
| | | | DTLCIVEAMKMMNQIEADKSGTVKAILV |
| | | | ESGQPVEFDEPLVVIE* |
| 3. | accC | pNH241 | MLDKIVIANRGEIALRILRACKELGIKT |
| | | | VAVHSSADRDLKHVLLADETVCIGPAPS |
| | | | VKSYLNIPAIISAAEITGAVAIHPGYGF |
| | | | LSENANFAEQVERSGFIFIGPKAETIRL |
| | | | MGDKVSAIAAMKKAGVPCVPGSDGPLGD |
| | | | DMDKNRAIAKRIGYPVIIKASGGGGGRG |
| | | | MRVVRGDAELAQSISMTRAEAKAAFSND |
| | | | MVYMEKYLENPRHVEIQVLADGQGNAIY |
| | | | LAERDCSMQRRHQKVVEEAPAPGITPEL |
| | | | RRYIGERCAKACVDIGYRGAGTFEFLFE |
| | | | NGEFYFIEMNTRIQVEHPVTEMITGVDL |
| | | | IKEQLRIAAGQPLSIKQEEVHVRGHAVE |
| | | | CRINAEDPNTFLPSPGKITRFHAPGGFG |
| | | | VRWESHIYAGYTVPPYYDSMIGKLICYG |
| | | | ENRDVAIARMKNALQELIIDGIKTNVDL |
| | | | QIRIMNDENFQHGGTNIHYLEKKLGLQE |
| | | | K* |
| 4. | accD | pNH241 | MSWIERIKSNITPTRKASIPEGVWTKCD |
| | | | SCGQVLYRAELERNLEVCPKCDHHMRMT |
| | | | ARNRLHSLLDEGSLVELGSELEPKDVLK |
| | | | FRDSKKYKDRLASAQKETGEKDALVVMK |
| | | | GTLYGMPVVAAAFEFAFMGGSMGSVVGA |
| | | | RFVRAVEQALEDNCPLICFSASGGARMQ |
| | | | EALMSLMQMAKTSAALAKMQERGLPYIS |
| | | | VLTDPTMGGVSASFAMLGDLNIAEPKAL |
| | | | IGFAGPRVIEQTVREKLPPGFQRSEFLI |
| | | | EKGAIDMIVRRPEMRLKLASILAKLMNL |
| | | | PAPNPEAPREGVVVPPVPDQEPEA* |
| 5. | mcrN | pNH243 | MSGTGRLAGKIALITGGAGNIGSELTRR |
| | | | FLAEGATVIISGRNRAKLTALAERMQAE |
| | | | AGVPAKRIDLEVMDGSDPVAVRAGIEAI |
| | | | VARHGQIDILVNNAGSAGAQRRLAEIPL |
| | | | TEAELGPGAEETLHASIANLLGMGWHLM |
| | | | RIAAPHMPVGSAVINVSTIFSRAEYYGR |
| | | | IPYVTPKAALNALSQLAARELGARGIRV |
| | | | NTIFPGPIESDRIRTVFQRMDQLKGRPE |
| | | | GDTAHHFLNTMRLCRANDQGALERRFPS |
| | | | VGDVADAAVFLASAESAALSGETIEVTH |
| | | | GMELPACSETSLLARTDLRTIDASGRTT |
| | | | LICAGDQIEEVMALTGMLRTCGSEVIIG |
| | | | FRSAAALAQFEQAVNESRRLAGADFTPP |
| | | | IALPLDPRDPATIDAVFDWGAGENTGGI |
| | | | HAAVILPATSHEPAPCVIEVDDERVLNF |
| | | | LADEITGTIVIASRLARYWQSQRLTPGA |
| | | | RARGPRVIFLSNGADQNGNVYGRIQSAA |
| | | | IGQLIRVWRHEAELDYQRASAAGDHVLP |
| | | | PVWANQIVRFANRSLEGLEFACAWTAQL |
| | | | LHSQRHINEITLNIPANI* |
| 6. | mcrC3 | pNH243 | MSATTGARSASVGWAESLIGLHLGKVAL |
| | | | ITGGSAGIGGQIGRLLALSGARVMLAAR |
| | | | DRHKLEQMQAMIQSELAEVGYTDVEDRV |
| | | | HIAPGCDVSSEAQLADLVERTLSAFGTV |
| | | | DYLINNAGIAGVEEMVIDMPVEGWRHTL |
| | | | FANLISNYSLMRKLAPLMKKQGSGYILN |
| | | | VSSYFGGEKDAAIPYPNRADYAVSKAGQ |
| | | | RAMAEVFARFLGPEIQINAIAPGPVEGD |
| | | | RLRGTGERPGLFARRARLILENKRLNEL |
| | | | HAALIAAARTDERSMHELVELLLPNDVA |
| | | | ALEQNPAAPTALRELARRFRSEGDPAAS |
| | | | SSSALLNRSIAAKLLARLHNGGYVLPAD |
| | | | IFANLPNPPDPFFTRAQIDREARKVRDG |
| | | | IMGMLYLQRMPTEFDVAMATVYYLADRV |
| | | | VSGETFHPSGGLRYERTPTGGELFGLPS |
| | | | PERLAELVGSTVYLIGEHLTEHLNLLAR |
| | | | AYLERYGARQVVMIVETETGAETMRRLL |
| | | | HDHVEAGRLMTIVAGDQIEAAIDQAITR |
| | | | YGRPGPVVCTPFRPLPTVPLVGRKDSDW |
| | | | STVLSEAEFAELCEHQLTHHFRVARWIA |
| | | | LSDGARLALVTPETTATSTTEQFALANF |
| | | | IKTTLHAFTATIGVESERTAQRILINQV |
| | | | DLTRRARAEEPRDPHERQQELERFIEAV |
| | | | LLVTAPLPPEADTRYAGRIHRGRAITV* |
| 7. | mcrC0 | | MSATTGARSASVGWAESLIGLHLGKVAL |
| | | | ITGGSAGIGGQIGRLLALSGARVMLAAR |
| | | | DRHKLEQMQAMIQSELAEVGYTDVEDRV |
| | | | HIAPGCDVSSEAQLADLVERTLSAFGTV |
| | | | DYLINNAGIAGVEEMVIDMPVEGWRHTL |
| | | | FANLISNYSLMRKLAPLMKKQGSGYILN |
| | | | VSSYFGGEKDAAIPYPNRADYAVSKAGQ |
| | | | RAMAEVFARFLGPEIQINAIAPGPVEGD |
| | | | RLRGTGERPGLFARRARLILENKRLNEL |
| | | | HAALIAAARTDERSMHELVELLLPNDVA |
| | | | ALEQNPAAPTALRELARRFRSEGDPAAS |
| | | | SSSALLNRSIAAKLLARLHNGGYVLPAD |
| | | | IFANLPNPPDPFFTRAQIDREARKVRDG |
| | | | IMGMLYLQRMPTEFDVAMATVYYLADRN |
| | | | VSGETFHPSGGLRYERTPTGGELFGLPS |
| | | | PERLAELVGSTVYLIGEHLTEHLNLLAR |
| | | | AYLERYGARQVVMIVETETGAETMRRLL |
| | | | HDHVEAGRLMTIVAGDQIEAAIDQAITR |
| | | | YGRPGPVVCTPFRPLPTVPLVGRKDSDW |
| | | | STVLSEAEFAELCEHQLTHHFRVARKIA |
| | | | LSDGASLALVTPETTATSTTEQFALANF |
| | | | IKTTLHAFTATIGVESERTAQRILINQV |
| | | | DLTRRARAEEPRDPHERQQELERFIEAV |
| | | | LLVTAPLPPEADTRYAGRIHRGRAITV* |
| 8. | mcr | | MSGTGRLAGKIALITGGAGNIGSELTRR |
| | | | FLAEGATVIISGRNRAKLTALAERMQAE |
| | | | AGVPAKRIDLEVMDGSDPVAVRAGIEAI |
| | | | VARHGQIDILVNNAGSAGAQRRLAEIPL |
| | | | TEAELGPGAEETLHASIANLLGMGWHLM |
| | | | RIAAPHMPVGSAVINVSTIFSRAEYYGR |
| | | | IPYVTPKAALNALSQLAARELGARGIRV |
| | | | NTIFPGPIESDRIRTVFQRMDQLKGRPE |
| | | | GDTAHHFLNTMRLCRANDQGALERRFPS |
| | | | VGDVADAAVFLASAESAALSGETIEVTH |
| | | | GMELPACSETSLLARTDLRTIDASGRTT |
| | | | LICAGDQIEEVMALTGMLRTCGSEVIIG |
| | | | FRSAAALAQFEQAVNESRRLAGADFTPP |
| | | | IALPLDPRDPATIDAVFDWGAGENTGGI |
| | | | HAAVILPATSHEPAPCVIEVDDERVLNF |
| | | | LADEITGTIVIASRLARYWQSQRLTPGA |
| | | | RARGPRVIFLSNGADQNGNVYGRIQSAA |
| | | | IGQLIRVWRHEAELDYQRASAAGDHVLP |
| | | | PVWANQIVRFANRSLEGLEFACAWTAQL |
| | | | LHSQRHINEITLNIPANISATTGARSAS |
| | | | VGWAESLIGLHLGKVALITGGSAGIGGQ |
| | | | IGRLLALSGARVMLAARDRHKLEQMQAM |
| | | | IQSELAEVGYTDVEDRVHIAPGCDVSSE |
| | | | AQLADLVERTLSAFGTVDYLINNAGIAG |
| | | | VEEMVIDMPVEGWRHTLFANLISNYSLM |
| | | | RKLAPLMKKQGSGYILNVSSYFGGEKDA |
| | | | AIPYPNRADYAVSKAGQRAMAEVFARFL |
| | | | GPEIQINAIAPGPVEGDRLRGTGERPGL |
| | | | FARRARLILENKRLNELHAALIAAARTD |
| | | | ERSMHELVELLLPNDVAALEQNPAAPTA |
| | | | LRELARRFRSEGDPAASSSSALLNRSIA |
| | | | AKLLARLHNGGYVLPADIFANLPNPPDP |
| | | | FFTRAQIDREARKVRDGIMGMLYLQRMP |
| | | | TEFDVAMATVYYLADRNVSGETFHPSGG |
| | | | LRYERTPTGGELFGLPSPERLAELVGST |
| | | | VYLIGEHLTEHLNLLARAYLERYGARQV |
| | | | VMIVETETGAETMRRLLHDHVEAGRLMT |
| | | | IVAGDQIEAAIDQAITRYGRPGPVVCTP |
| | | | FRPLPTVPLVGRKDSDWSTVLSEAEFAE |
| | | | LCEHQLTHHFRVARKIALSDGASLALVT |
| | | | PETTATSTTEQFALANFIKTTLHAFTAT |
| | | | IGVESERTAQRILINQVDLTRRARAEEP |
| | | | RDPHERQQELERFIEAVLLVTAPLPPEA |
| | | | DTRYAGRIHRGRAITV* |
| 9. | mmoX | pNH265 | MALSTATKAATDALAANRAPTSVNAQEV |
| | | | HRWLQSFNWDFKNNRTKYATKYKMANET |
| | | | KEQFKLIAKEYARMEAVKDERQFGSLQD |
| | | | ALTRLNAGVRVHPKWNETMKVVSNFLEV |
| | | | GEYNAIAATGMLWDSAQAAEQKNGYLAQ |
| | | | VLDEIRHTHQCAYVNYYFAKNGQDPAGH |
| | | | NDARRTRTIGPLWKGMKRVFSDGFISGD |
| | | | AVECSLNLQLVGEACFTNPLIVAVTEWA |
| | | | AANGDEITPTVFLSIETDELRHMANGYQ |
| | | | TVVSIANDPASAKYLNTDLNNAFWTQQK |
| | | | YFTPVLGMLFEYGSKFKVEPWVKTWNRW |
| | | | VYEDWGGIWIGRLGKYGVESPRSLKDAK |
| | | | QDAYWAHHDLYLLAYALWPTGFFRLALP |
| | | | DQEEMEWFEANYPGWYDHYGKIYEEWRA |
| | | | RGCEDPSSGFIPLMWFIENNHPIYIDRV |
| | | | SQVPFCPSLAKGASTLRVHEYNGQMHTF |
| | | | SDQWGERMWLAEPERYECQNIFEQYEGR |
| | | | ELSEVIAELHGLRSDGKTLIAQPHVRGD |
| | | | KLWTLDDIKRLNCVFKNPVKAFN* |
| 10. | mmoY | pNH265 | MSMLGERRRGLTDPEMAAVILKALPEAP |
| | | | LDGNNKMGYFVTPRWKRLTEYEALTVYA |
| | | | QPNADWIAGGLDWGDWTQKFHGGRPSWG |
| | | | NETTELRTVDWFKHRDPLRRWHAPYVKD |
| | | | KAEEWRYTDRFLQGYSADGQIRAMNPTW |
| | | | RDEFINRYWGAFLFNEYGLFNAHSQGAR |
| | | | EALSDVTRVSLAFWGFDKIDIAQMIQLE |
| | | | RGFLAKIVPGFDESTAVPKAEWTNGEVY |
| | | | KSARLAVEGLWQEVFDWNESAFSVHAVY |
| | | | DALFGQFVRREFFQRLAPRFGDNLTPFF |
| | | | INQAQTYFQIAKQGVQDLYYNCLGDDPE |
| | | | FSDYNRTVMRNWTGKWLEPTIAALRDFM |
| | | | GLFAKLPAGTTDKEEITASLYRVVDDWI |
| | | | EDYASRIDFKADRDQIVKAVLAGLK* |
| 11. | mmoB | pNH265 | MSVNSNAYDAGIMGLKGKDFADQFFADE |
| | | | NQVVHESDTVVLVLKKSDEINTFIEEIL |
| | | | LTDYKKNVNPTVNVEDRAGYWWIKANGK |
| | | | IEVDCDEISELLGRQFNVYDFLVDVSST |
| | | | IGRAYTLGNKFTITSELMGLDRKLEDYH |
| | | | A* |
| 12. | mmoZ | pNH265 | MAKLGIHSNDTRDAWVNKIAQLNTLEKA |
| | | | AEMLKQFRMDHTTPFRNSYELDNDYLWI |
| | | | EAKLEEKVAVLKARAFNEVDFRHKTAFG |
| | | | EDAKSVLDGTVAKMNAAKDKWEAEKIMT |
| | | | GFRQAYKPPIMPVNYFLDGERQLGTRLM |
| | | | ELRNLNYYDTPLEELRKQRGVRVVHLQS |
| | | | PH* |
| 13. | mmoC | pNH265 | MQRVHTITAVTEDGESLRFECRSDEDVI |
| | | | TAALRQNIFLMSSCREGGCATCKALCSE |
| | | | GDYDLKGCSVQALPPEEEEEGLVLLCRT |
| | | | YPKTDLEIELPYTHCRISFGEVGSFEAE |
| | | | VVGLNWVSSNTVQFLLQKRPDECGNRGV |
| | | | KFEPGQFMDLTIPGTDVSRSYSPANLPN |
| | | | PEGRLEFLIRVLPEGRFSDYLRNDARVG |
| | | | QVLSVKGPLGVFGLKERGMAPRYFVAGG |
| | | | TGLAPVVSMVRQMQEWTAPNETRIYFGV |
| | | | NTEPELFYIDELKSLERSMRNLTVKACV |
| | | | WHPSGDWEGEQGSPIDALREDLESSDAN |
| | | | PDIYLCGPPGMIDAACELVRSRGIPGEQ |
| | | | VFFEKFLPSGAA* |
| 14. | mmoD | pNH265 | MVESAFQPFSGDADEWFEEPRPQAGFFP |
| | | | SADWHLLKRDETYAAYAKDLDFMWRWVI |
| | | | VREERIVQEGCSISLESSIRAVTHVLNY |
| | | | FGMTEQRAPAEDRTGGVQH* |
| 15. | groEL- | pNH265 | MAKEVVYRGSARQRMMQGIEILARAAIP |
| | 2 | | TLGATGPSVMIQHRADGLPPISTRDGVT |
| | | | VANSIVLKDRVANLGARLLRDVAGTMSR |
| | | | EAGDGTTTAIVLARHIAREMFKSLAVGA |
| | | | DPIALKRGIDRAVARVSEDIGARAWRGD |
| | | | KESVILGVAAVATKGEPGVGRLLLEALD |
| | | | AVGVHGAVSIELGQRREDLLDVVDGYRW |
| | | | EKGYLSPYFVTDRARELAELEDVYLLMT |
| | | | DREVVDFIDLVPLLEAVTEAGGSLLIAA |
| | | | DRVHEKALAGLLLNHVRGVFKAVAVTAP |
| | | | GFGDKRPNRLLDLAALTGGRAVLEAQGD |
| | | | RLDRVTLADLGRVRRAVVSADDTALLGI |
| | | | PGTEASRARLEGLRLEAEQYRALKPGQG |
| | | | SATGRLHELEEIEARIVGLSGKSAVYRV |
| | | | GGVTDVEMKERMVRIENAYRSVVSALEE |
| | | | GVLPGGGVGFLGSMPVLAELEARDADEA |
| | | | RGIGIVRSALTEPLRIIGENSGLSGEAV |
| | | | VAKVMDHANPGWGYDQESGSFCDLHARG |
| | | | IWDAAKVLRLALEKAASVAGTFLTTEAV |
| | | | VLEIPDTDAFAGFSAEWAAATREDPRV* |
| 16. | groES_ | pNH265 | VKIRPLHDRVIIKRLEEERTSAGGIVIP |
| | mc | | DSAAEKPMRGEILAVGNGKVLDNGEVRA |
| | | | LQVKVGDKVLFGKYAGTEVKVDGEDVVV |
| | | | MREDDILAVLES* |
| 17. | groES_ | pNH265 | MNIRPLHDRVIVKRKEVETKSAGGIVLT |
| | ec | | GSAAAKSTRGEVLAVGNGRILENGEVKP |
| | | | LDVKVGDIVIFNDGYGVKSEKIDNEEVL |
| | | | IMSESDILAIVEA* |
| 18. | groEL_ | pNH265 | MAAKDVKFGNDARVKMLRGVNVLADAVK |
| | ec | | VTLGPKGRNVVLDKSFGAPTITKDGVSV |
| | | | AREIELEDKFENMGAQMVKEVASKANDA |
| | | | AGDGTTTATVLAQAIITEGLKAVAAGMN |
| | | | PMDLKRGIDKAVTAAVEELKALSVPCSD |
| | | | SKAIAQVGTISANSDETVGKLIAEAMDK |
| | | | VGKEGVITVEDGTGLQDELDVVEGMQFD |
| | | | RGYLSPYFINKPETGAVELESPFILLAD |
| | | | KKISNIREMLPVLEAVAKAGKPLLIIAE |
| | | | DVEGEALATLVVNTMRGIVKVAAVKAPG |
| | | | FGDRRKAMLQDIATLTGGTVISEEIGME |
| | | | LEKATLEDLGQAKRVVINKDTTTIIDGV |
| | | | GEEAAIQGRVAQIRQQIEEATSDYDREK |
| | | | LQERVAKLAGGVAVIKVGAATEVEMKEK |
| | | | KARVEDALHATRAAVEEGVVAGGGVALI |
| | | | RVASKLADLRGQNEDQNVGIKVALRAME |
| | | | APLRQIVLNCGEEPSVVANTVKGGDGNY |
| | | | GYNAATEEYGNMIDMGILDPTKVTRSAL |
| | | | QYAASVAGLMITTECMVTDLPKNDAADL |
| | | | GAAGGMGGMM* |
| 19. | HPS | pLC130 | MELQLALDLVNIEEAKQVVAEVQEYVDI |
| | | | VEIGTPVIKIWGLQAVKAVKDAFPHLQV |
| | | | LADMKTMDAAAYEVAKAAEHGADIVTIL |
| | | | AAAEDVSIKGAVEEAKKLGKKILVDMIA |
| | | | VKNLEERAKQVDEMGVDYICVHAGYDLQ |
| | | | AVGKNPLDDLKRIKAVVKNAKTAIAGGI |
| | | | KLETLPEVIKAEPDLVIVGGGIANQTDK |
| | | | KAAAEKINKLVKQGL* |
| 20. | PHI | pLC130 | MISMLTTEFLAEIVKELNSSVNQIADEE |
| | | | AEALVNGILQSKKVFVAGAGRSGFMAKS |
| | | | FAMRMMHMGIDAYVVGETVTPNYEKEDI |
| | | | LIIGSGSGETKSLVSMAQKAKSIGGTIA |
| | | | AVTINPESTIGQLADIVIKMPGSPKDKS |
| | | | EARETIQPMGSLFEQTLLLFYDAVILRF |
| | | | MEKKGLDTKTMYGRHANLE* |
| 21. | mdh2_ | pLC130 | MTNTQSAFFMPSVNLFGAGSVNEVGTRL |
| | Bm | | ADLGVKKALLVTDAGLHGLGLSEKISSI |
| | | | IRAAGVEVSIFPKAEPNPTDKNVAEGLE |
| | | | AYNAENCDSIVTLGGGSSHDAGKAIALV |
| | | | AANGGKIHDYEGVDVSKEPMVPLTAINT |
| | | | TAGTGSELTKFTIITDTERKVKMAIVDK |
| | | | HVTPTLSINDPELMVGMPPSLTAATGLD |
| | | | ALTHAIEAYVSTGATPITDALAIQAIKI |
| | | | ISKYLPRAVANGKDIEAREQMAFAQSLA |
| | | | GMAFNNAGLGYVHAIAHQLGGFYNFPHG |
| | | | VCNAVLLPYVCRFNLISKVERYAEIAAF |
| | | | LGENVDGLSTYDAAEKAIKAIERMAKDL |
| | | | NIPKGFKELGAKEEDIETLAKNAMKDAC |
| | | | ALTNPRKPKLEEVIQIIKNAM* |
| 22. | HPS | pLC158 | MELQLALDLVNIEEAKQVVAEVQEYVDI |
| | | | VEIGTPVIKIWGLQAVKAVKDAFPHLQV |
| | | | LADMKTMDAAAYEVAKAAEHGADIVTIL |
| | | | AAAEDVSIKGAVEEAKKLGKKILVDMIA |
| | | | VKNLEERAKQVDEMGVDYICVHAGYDLQ |
| | | | AVGKNPLDDLKRIKAVVKNAKTAIAGGI |
| | | | KLETLPEVIKAEPDLVIVGGGIANQTDK |
| | | | KAAAEKINKLVKQGL* |
| 23. | PHI | pLC158 | MISMLTTEFLAEIVKELNSSVNQIADEE |
| | | | AEALVNGILQSKKVFVAGAGRSGFMAKS |
| | | | FAMRMMHMGIDAYVVGETVTPNYEKEDI |
| | | | LIIGSGSGETKSLVSMAQKAKSIGGTIA |
| | | | AVTINPESTIGQLADIVIKMPGSPKDKS |
| | | | EARETIQPMGSLFEQTLLLFYDAVILRF |
| | | | MEKKGLDTKTMYGRHANLE* |
| 24. | adhA_ | pLC158 | MTTAAPQEFTAAVVEKFGHDVTVKDIDL |
| | CG | | PKPGPHQALVKVLTSGICHTDLHALEGD |
| | | | WPVKPEPPFVPGHEGVGEVVELGPGEHD |
| | | | VKVGDIVGNAWLWSACGTCEYCITGRET |
| | | | QCNEAEYGGYTQNGSFGQYMLVDTRYAA |
| | | | RIPDGVDYLEAAPILCAGVTVYKALKVS |
| | | | ETRPGQFMVISGVGGLGHIAVQYAAAMG |
| | | | MRVIAVDIADDKLELARKHGAEFTVNAR |
| | | | NEDSGEAVQKYTNGGAHGVLVTAVHEAA |
| | | | FGQALDMARRAGTIVFNGLPPGEFPASV |
| | | | FNIVFKGLTIRGSLVGTRQDLAEALDFF |
| | | | ARGLIKPTVSECSLDEVNGVLDRMRNGK |
| | | | IDGRVAIRY* |
| 25. | mdh2_ | pBZ27 | MTNTQSAFFMPSVNLFGAGSVNEVGTRL |
| | Bm | | ADLGVKKALLVTDAGLHGLGLSEKISSI |
| | | | IRAAGVEVSIFPKAEPNPTDKNVAEGLE |
| | | | AYNAENCDSIVTLGGGSSHDAGKAIALV |
| | | | AANGGKIHDYEGVDVSKEPMVPLTAINT |
| | | | TAGTGSELTKFTIITDTERKVKMAIVDK |
| | | | HVTPTLSINDPELMVGMPPSLTAATGLD |
| | | | ALTHAIEAYVSTGATPITDALAIQAIKI |
| | | | ISKYLPRAVANGKDIEAREQMAFAQSLA |
| | | | GMAFNNAGLGYVHAIAHQLGGFYNFPHG |
| | | | VCNAVLLPYVCRFNLISKVERYAEIAAF |
| | | | LGENVDGLSTYDAAEKAIKAIERMAKDL |
| | | | NIPKGFKELGAKEEDIETLAKNAMKDAC |
| | | | ALTNPRKPKLEEVIQIIKNAM* |
| 26. | mdh_Bm | pBZ27 | MTTNFFIPPASVIGRGAVKEVGTRLKQI |
| | | | GAKKALIVTDAFLHSTGLSEEVAKNIRE |
| | | | AGVDVAIFPKAQPDPADTQVHEGVDVFK |
| | | | QENCDSLVSIGGGSSHDTAKAIGLVAAN |
| | | | GGRINDYQGVNSVEKPVVPVVAITTTAG |
| | | | TGSETTSLAVITDSARKVKMPVIDEKIT |
| | | | PTVAIVDPELMVKKPAGLTIATGMDALS |
| | | | HAIEAYVAKGATPVTDAFAIQAMKLINE |
| | | | YLPKAVANGEDIEAREKMAYAQYMAGVA |
| | | | FNNGGLGLVHSISHQVGGVYKLQHGICN |
| | | | SVNMPHVCAFNLIAKTERFAHIAELLGE |
| | | | NVAGLSTAAAAERAIVALERINKSFGIP |
| | | | SGYAEMGVKEEDIELLAKNAYEDVCTQS |
| | | | NPRVPTVQDIAQIIKNAM* |
| 27. | HPS | pBZ27 | MELQLALDLVNIEEAKQVVAEVQEYVDI |
| | | | VEIGTPVIKIWGLQAVKAVKDAFPHLQV |
| | | | LADMKTMDAAAYEVAKAAEHGADIVTIL |
| | | | AAAEDVSIKGAVEEAKKLGKKILVDMIA |
| | | | VKNLEERAKQVDEMGVDYICVHAGYDLQ |
| | | | AVGKNPLDDLKRIKAVVKNAKTAIAGGI |
| | | | KLETLPEVIKAEPDLVIVGGGIANQTDK |
| | | | KAAAEKINKLVKQGL* |
| 28. | PHI | pBZ27 | MISMLTTEFLAEIVKELNSSVNQIADEE |
| | | | AEALVNGILQSKKVFVAGAGRSGFMAKS |
| | | | FAMRMMHMGIDAYVVGETVTPNYEKEDI |
| | | | LIIGSGSGETKSLVSMAQKAKSIGGTIA |
| | | | AVTINPESTIGQLADIVIKMPGSPKDKS |
| | | | EARETIQPMGSLFEQTLLLFYDAVILRF |
| | | | MEKKGLDTKTMYGRHANLE* |
| 29. | rpeP | pBZ27 | MIKIAPSILSANFARLEEEIKDVERGGA |
| | | | DYIHVDVMDGHFVPNITIGPLIVEAIRP |
| | | | VTNLPLDVHLMIENPDQYIGTFAKAGAD |
| | | | ILSVHVEACTHLHRTIQYIKSEGIKAGV |
| | | | VLNPHTPVSMIEHVIEDVDLVLLMTVNP |
| | | | GFGGQSFIHSVLPKIKQVANIVKEKNLQ |
| | | | VEIEVDGGVNPETAKLCVEAGANVLVAG |
| | | | SAIYNQEDRSQAIAKIRN* |
| 30. | glpXP | pBZ27 | MRELKSEKRVQSLAMEFLSVAQQAALAS |
| | | | YPWIGKGNKNEVDRAGTEAMRNRLNLID |
| | | | MSGLIVIGEGEMDEAPMLYIGEELGTGK |
| | | | GPQLDIAVDPVDGTGLMAKGMDNSIAVI |
| | | | AASTRGSLLHAPDMYMEKIAVGPKAKGC |
| | | | VNLDASLTENMKSVAKALGKDLRELTVM |
| | | | IQDRPRHDHLIQQVRDVGARLKLFSDGD |
| | | | VTRAIGTALEEVDVDILVGTGGAPEGVI |
| | | | AATALKCLGGDFQGRLAPQNEEEFDRCI |
| | | | TMGITDPRKIFTIDEIVKSDDCFFVATG |
| | | | ITDGLLINGIRKKEDGLMQTHSFLTIGG |
| | | | SSVKYQFIEAYH* |
| 31. | fbaP | pBZ27 | MPLVSMKDMLNHGKENGYAVGQFNINNL |
| | | | EFGQAILQAAEEEKSPVIIGVSVGAANY |
| | | | MGGFKLIVDMVKSLMDSYNVTVPVAIHL |
| | | | DHGPSLEKCVQAIHAGFTSVMIDGSHLP |
| | | | LEENIELTKRVVEIAHSVGVSVEAELGR |
| | | | IGGQEDDVVAESFYAIPSECEQLVRETG |
| | | | VDCFAPALGSVHGPYKGEPKLGFDRMEE |
| | | | IMKLTGVPLVLHGGTGIPTKDIQKAISL |
| | | | GTAKINVNTESQIAATKAVREVLNNDAK |
| | | | LFDPRKFLAPAREAIKETIKGKMREFGS |
| | | | SGKA* |
| 32. | tktP | pBZ27 | VLQQKIDIDQLSIQTIRTLSIDAIEKVG |
| | | | SGHPGMPMGAAPMAYTLWTKFMNYNPSN |
| | | | PNWFNRDRFVLSAGHGSMLLYSLLHLTG |
| | | | YDLSLEDLKNFRQWGSKTPGHPEFGHTP |
| | | | GVDATTGPLGQGIAMAVGMAMAERHLAS |
| | | | KYNRYKFNIIDHYTYSICGDGDLMEGVS |
| | | | AEAASLAGHLKLGRLIVLYDSNDISLDG |
| | | | DLHMSFSESVQDRFKAYGWQVLRVEDGN |
| | | | DIDSIAKAIAEAKNNEDQPTLIEVKTII |
| | | | GYGSPNKGGKSDAHGSPLGKEEIKLVKE |
| | | | HYNWKYDEDFYIPEEVKEYFRELKEAAE |
| | | | KKEQAWNELFAQYKEAYPALAKELEQAI |
| | | | NGELPEGWDADVPVYRVGEDKLATRSSS |
| | | | GAVLNALAKNVPQLLGGSADLASSNKTL |
| | | | LKGEANFSATDYSGRNIWFGVREFGMGA |
| | | | AVNGMALHGGVKVFGATFFVFSDYLRPA |
| | | | IRLSALMKLPVIYVFTHDSVAVGEDGPT |
| | | | HEPIEQLASLRAMPGISTIRPADGNETA |
| | | | AAWKLALESKDEPTALILSRQDLPTLVD |
| | | | SEKAYEGVKKGAYVISEAKGEVAGLLLA |
| | | | SGSEVALAVEAQAALEKEGIYVSVVSMP |
| | | | SWDRFEKQSDAYKESVLPKNVKARLGIE |
| | | | MGASLGWSKYVGDNGNVLAIDQFGSSAP |
| | | | GDKIIEEYGFTVENVVSHFKKLL* |
| 33. | pfkP | pBZ27 | MNKIAVLTSGGDAPGMNAAIRAVVRRGI |
| | | | FKGLDVYGVKNGYKGLMNGNFVSMNLGS |
| | | | VGDIIHRGGTILQTTRCKEFKTAEGQQQ |
| | | | ALAQLKKEGIDGLIVIGGDGTFEGARKL |
| | | | TAQEFPTIGIPATIDNDIAGTEYTIGFD |
| | | | TAVNTAVEAIDKIRDTAASHDRIYVVEV |
| | | | MGRNAGDIALWAGMCAGAESIIIPEADH |
| | | | DVEDVIDRIKQGYQRGKTHSIIVVAEGA |
| | | | FNGVGAIEIGRAIKEKTGFDTKVTILGH |
| | | | IQRGGSPSAYDRMMSSQMGAKAVDLLVE |
| | | | GKKGLMVGLKNGQLIHTPFEEAAKDKHT |
| | | | VDLSIYHLARSLSL* |
| 34. | | pNH241 | TAATGTGTAAAACATGTACATGCAGATT |
| | | | GCTGGGGGTGCAGGGGGCGGAGCCACCC |
| | | | TGTCCATGCGGGGTGTGGGGCTTGCCCC |
| | | | GCCGGTACAGACAGTGAGCACCGGGGCA |
| | | | CCTAGTCGCGGATACCCCCCCTAGGTAT |
| | | | CGGACACGTAACCCTCCCATGTCGATGC |
| | | | AAATCTTTAACATTGAGTACGGGTAAGC |
| | | | TGGCACGCATAGCCAAGCTAGGCGGCCA |
| | | | CCAAACACCACTAAAAATTAATAGTCCC |
| | | | TAGACAAGACAAACCCCCGTGCGAGCTA |
| | | | CCAACTCATATGCACGGGGGCCACATAA |
| | | | CCCGAAGGGGTTTCAATTGACAACCATA |
| | | | GCACTAGCTAAGACAACGGGCACAACAC |
| | | | CCGCACAAACTCGCACTGCGCAACCCCG |
| | | | CACAACATCGGGTCTAGGTAACACTGAA |
| | | | ATAGAAGTGAACACCTCTAAGGAACCGC |
| | | | AGGTCAATGAGGGTTCTAAGGTCACTCG |
| | | | CGCTAGGGCGTGGCGTAGGCAAAACGTC |
| | | | ATGTACAAGATCACCAATAGTAAGGCTC |
| | | | TGGCGGGGTGCCATAGGTGGCGCAGGGA |
| | | | CGAAGCTGTTGCGGTGTCCTGGTCGTCT |
| | | | AACGGTGCTTCGCAGTTTGAGGGTCTGC |
| | | | AAAACTCTCACTCTCGCTGGGGGTCACC |
| | | | TCTGGCTGAATTGGAAGTCATGGGCGAA |
| | | | CGCCGCATTGAGCTGGCTATTGCTACTA |
| | | | AGAATCACTTGGCGGCGGGTGGCGCGCT |
| | | | CATGATGTTTGTGGGCACTGTTCGACAC |
| | | | AACCGCTCACAGTCATTTGCGCAGGTTG |
| | | | AAGCGGGTATTAAGACTGCGTACTCTTC |
| | | | GATGGTGAAAACATCTCAGTGGAAGAAA |
| | | | GAACGTGCACGGTACGGGGTGGAGCACA |
| | | | CCTATAGTGACTATGAGGTCACAGACTC |
| | | | TTGGGCGAACGGTTGGCACTTGCACCGC |
| | | | AACATGCTGTTGTTCTTGGATCGTCCAC |
| | | | TGTCTGACGATGAACTCAAGGCGTTTGA |
| | | | GGATTCCATGTTTTCCCGCTGGTCTGCT |
| | | | GGTGTGGTTAAGGCCGGTATGGACGCGC |
| | | | CACTGCGTGAGCACGGGGTCAAACTTGA |
| | | | TCAGGTGTCTACCTGGGGTGGAGACGCT |
| | | | GCGAAAATGGCAACCTACCTCGCTAAGG |
| | | | GCATGTCTCAGGAACTGACTGGCTCCGC |
| | | | TACTAAAACCGCGTCTAAGGGGTCGTAC |
| | | | ACGCCGTTTCAGATGTTGGATATGTTGG |
| | | | CCGATCAAAGCGACGCCGGCGAGGATAT |
| | | | GGACGCTGTTTTGGTGGCTCGGTGGCGT |
| | | | GAGTATGAGGTTGGTTCTAAAAACCTGC |
| | | | GTTCGTCCTGGTCACGTGGGGCTAAGCG |
| | | | TGCTTTGGGCATTGATTACATAGACGCT |
| | | | GATGTACGTCGTGAAATGGAAGAAGAAC |
| | | | TGTACAAGCTCGCCGGTCTGGAAGCACC |
| | | | GGAACGGGTCGAATCAACCCGCGTTGCT |
| | | | GTTGCTTTGGTGAAGCCCGATGATTGGA |
| | | | AACTGATTCAGTCTGATTTCGCGGTTAG |
| | | | GCAGTACGTTCTAGATTGCGTGGATAAG |
| | | | GCTAAGGACGTGGCCGCTGCGCAACGTG |
| | | | TCGCTAATGAGGTGCTGGCAAGTCTGGG |
| | | | TGTGGATTCCACCCCGTGCATGATCGTT |
| | | | ATGGATGATGTGGACTTGGACGCGGTTC |
| | | | TGCCTACTCATGGGGACGCTACTAAGCG |
| | | | TGATCTGAATGCGGCGGTGTTCGCGGGT |
| | | | AATGAGCAGACTATTCTTCGCACCCACT |
| | | | AAAAGCGGCATAAACCCCGTTCGATATT |
| | | | TTGTGCGATGAATTTATGGTCAATGTCG |
| | | | CGGGGGCAAACTATGATGGGTCTTGTTG |
| | | | TTGCAGCCGAACGACCTAGCGCAGCGAG |
| | | | TCAGTGAGCGAGGAAGCGGAAGAGCGCC |
| | | | TGATGCGGTATTTTCTCCTTACGCATCT |
| | | | GTGCGGTATTTCACACCGCATATGGTGC |
| | | | ACTCTCAGTACAATCTGCTCTGATGCCG |
| | | | CATAGTTAAGCCAGTATACACTCCGCTA |
| | | | TCGCTACGTGACTGGGTCATGGCTGCGC |
| | | | CCCGACACCCGCCAACACCCGCTGACGC |
| | | | GCCCTGACGGGCTTGTCTGCTCCCGGCA |
| | | | TCCGCTTACAGACAAGCTGTGACCGTCT |
| | | | CCGGGAGCTGCATGTGTCAGAGGTTTTC |
| | | | ACCGTCATCACCGAAACGCGCGAGGCAG |
| | | | CAGATCAATTCGCGCGCGAAGGCGAAGC |
| | | | GGCATGCATAATGTGCCTGTCAAATGGA |
| | | | CGAAGCAGGGATTCTGCAAACCCTATGC |
| | | | TACTCCGTCAAGCCGTCAATTGTCTGAT |
| | | | TCGTTACCAATTATGACAACTTGACGGC |
| | | | TACATCATTCACTTTTTCTTCACAACCG |
| | | | GCACGGAACTCGCTCGGGCTGGCCCCGG |
| | | | TGCATTTTTTAAATACCCGCGAGAAATA |
| | | | GAGTTGATCGTCAAAACCAACATTGCGA |
| | | | CCGACGGTGGCGATAGGCATCCGGGTGG |
| | | | TGCTCAAAAGCAGCTTCGCCTGGCTGAT |
| | | | ACGTTGGTCCTCGCGCCAGCTTAAGACG |
| | | | CTAATCCCTAACTGCTGGCGGAAAAGAT |
| | | | GTGACAGACGCGACGGCGACAAGCAAAC |
| | | | ATGCTGTGCGACGCTGGCGATATCAAAA |
| | | | TTGCTGTCTGCCAGGTGATCGCTGATGT |
| | | | ACTGACAAGCCTCGCGTACCCGATTATC |
| | | | CATCGGTGGATGGAGCGACTCGTTAATC |
| | | | GCTTCCATGCGCCGCAGTAACAATTGCT |
| | | | CAAGCAGATTTATCGCCAGCAGCTCCGA |
| | | | ATAGCGCCCTTCCCCTTGCCCGGCGTTA |
| | | | ATGATTTGCCCAAACAGGTCGCTGAAAT |
| | | | GCGGCTGGTGCGCTTCATCCGGGCGAAA |
| | | | GAACCCCGTATTGGCAAATATTGACGGC |
| | | | CAGTTAAGCCATTCATGCCAGTAGGCGC |
| | | | GCGGACGAAAGTAAACCCACTGGTGATA |
| | | | CCATTCGCGAGCCTCCGGATGACGACCG |
| | | | TAGTGATGAATCTCTCCTGGCGGGAACA |
| | | | GCAAAATATCACCCGGTCGGCAAACAAA |
| | | | TTCTCGTCCCTGATTTTTCACCACCCCC |
| | | | TGACCGCGAATGGTGAGATTGAGAATAT |
| | | | AACCTTTCATTCCCAGCGGTCGGTCGAT |
| | | | AAAAAAATCGAGATAACCGTTGGCCTCA |
| | | | ATCGGCGTTAAACCCGCCACCAGATGGG |
| | | | CATTAAACGAGTATCCCGGCAGCAGGGG |
| | | | ATCATTTTGCGCTTCAGCCATACTTTTC |
| | | | ATACTCCCGCCATTCAGAGAAGAAACCA |
| | | | ATTGTCCATATTGCATCAGACATTGCCG |
| | | | TCACTGCGTCTTTTACTGGCTCTTCTCG |
| | | | CTAACCAAACCGGTAACCCCGCTTATTA |
| | | | AAAGCATTCTGTAACAAAGCGGGACCAA |
| | | | AGCCATGACAAAAACGCGTAACAAAAGT |
| | | | GTCTATAATCACGGCAGAAAAGTCCACA |
| | | | TTGATTATTTGCACGGCGTCACACTTTG |
| | | | CTATGCCATAGCATTTTTATCCATAAGA |
| | | | TTAGCGGATCCTACCTGACGCTTTTTAT |
| | | | CGCAACTCTCTACTGTTTCTCCATACCC |
| | | | GTTTTTTTGGGATCTCGAGGGTGTTTTC |
| | | | ACGAGCAATTGACCAACAAGGACAGGAG |
| | | | GCCTAATGAGCTGGATTGAACGAATTAA |
| | | | AAGCAACATTACTCCCACCCGCAAGGCG |
| | | | AGCATTCCTGAAGGGGTGTGGACTAAGT |
| | | | GTGATAGCTGCGGTCAGGTTTTATACCG |
| | | | CGCTGAGCTGGAACGTAATCTTGAGGTC |
| | | | TGTCCGAAGTGTGACCATCACATGCGTA |
| | | | TGACAGCGCGTAATCGCCTGCATAGCCT |
| | | | GTTAGATGAAGGAAGCCTTGTGGAGCTG |
| | | | GGTAGCGAGCTTGAGCCGAAAGATGTGC |
| | | | TGAAGTTTCGTGACTCCAAGAAGTATAA |
| | | | AGACCGTCTGGCATCTGCGCAGAAAGAA |
| | | | ACCGGCGAAAAAGATGCGCTGGTGGTGA |
| | | | TGAAAGGCACTCTGTATGGAATGCCGGT |
| | | | TGTCGCTGCGGCATTCGAGTTCGCCTTT |
| | | | ATGGGCGGTTCAATGGGGTCTGTTGTGG |
| | | | GTGCACGTTTCGTGCGTGCCGTTGAGCA |
| | | | GGCGCTGGAAGATAACTGCCCGCTGATC |
| | | | TGCTTCTCCGCCTCTGGTGGCGCACGTA |
| | | | TGCAGGAAGCACTGATGTCGCTGATGCA |
| | | | GATGGCGAAAACCTCTGCGGCACTGGCA |
| | | | AAAATGCAGGAGCGCGGCTTGCCGTACA |
| | | | TCTCCGTGCTGACCGACCCGACGATGGG |
| | | | CGGTGTTTCTGCAAGTTTCGCCATGCTG |
| | | | GGCGATCTCAACATCGCTGAACCGAAAG |
| | | | CGTTAATCGGCTTTGCCGGTCCGCGTGT |
| | | | TATCGAACAGACCGTTCGCGAAAAACTG |
| | | | CCGCCTGGATTCCAGCGCAGTGAATTCC |
| | | | TGATCGAGAAAGGCGCGATCGACATGAT |
| | | | CGTCCGTCGTCCGGAAATGCGCCTGAAA |
| | | | CTGGCGAGCATTCTGGCGAAGTTGATGA |
| | | | ATCTGCCAGCGCCGAATCCTGAAGCGCC |
| | | | GCGTGAAGGCGTAGTGGTACCCCCGGTA |
| | | | CCGGATCAGGAACCTGAGGCCTGATTAG |
| | | | GAGGTTAATATGAGTCTGAATTTCCTTG |
| | | | ATTTTGAACAGCCGATTGCAGAGCTGGA |
| | | | AGCGAAAATCGATTCTCTGACTGCGGTT |
| | | | AGCCGTCAGGATGAGAAACTGGATATTA |
| | | | ACATCGATGAAGAAGTGCATCGTCTGCG |
| | | | TGAAAAAAGCGTAGAACTGACACGTAAA |
| | | | ATCTTCGCCGATCTCGGTGCATGGCAGA |
| | | | TTGCGCAACTGGCACGCCATCCACAGCG |
| | | | TCCTTATACCCTGGATTACGTTCGCCTG |
| | | | GCATTTGATGAATTTGACGAACTGGCTG |
| | | | GCGACCGCGCGTATGCAGACGATAAAGC |
| | | | TATCGTCGGTGGTATCGCCCGTCTCGAT |
| | | | GGTCGTCCGGTGATGATCATTGGTCATC |
| | | | AAAAAGGTCGTGAAACCAAAGAAAAAAT |
| | | | TCGCCGTAACTTTGGTATGCCAGCGCCA |
| | | | GAAGGTTACCGCAAAGCACTGCGTCTGA |
| | | | TGCAAATGGCTGAACGCTTTAAGATGCC |
| | | | TATCATCACCTTTATCGACACCCCGGGG |
| | | | GCTTATCCTGGCGTGGGCGCAGAAGAGC |
| | | | GTGGTCAGTCTGAAGCCATTGCACGCAA |
| | | | CCTGCGTGAAATGTCTCGCCTCGGCGTA |
| | | | CCGGTAGTTTGTACGGTTATCGGTGAAG |
| | | | GTGGTTCTGGCGGTGCGCTGGCGATTGG |
| | | | CGTGGGCGATAAAGTGAATATGCTGCAA |
| | | | TACAGCACCTATTCCGTTATCTCGCCGG |
| | | | AAGGTTGTGCGTCCATTCTGTGGAAGAG |
| | | | CGCCGACAAAGCGCCGCTGGCGGCTGAA |
| | | | GCGATGGGTATCATTGCTCCGCGTCTGA |
| | | | AAGAACTGAAACTGATCGACTCCATCAT |
| | | | CCCGGAACCACTGGGTGGTGCTCACCGT |
| | | | AACCCGGAAGCGATGGCGGCATCGTTGA |
| | | | AAGCGCAACTGCTGGCGGATCTGGCCGA |
| | | | TCTCGACGTGTTAAGCACTGAAGATTTA |
| | | | AAAAATCGTCGTTATCAGCGCCTGATGA |
| | | | GCTACGGTTACGCGTAAAAAGGAGAATA |
| | | | TATGGATATTCGTAAGATTAAAAAACTG |
| | | | ATCGAGCTGGTTGAAGAATCAGGCATCT |
| | | | CCGAACTGGAAATTTCTGAAGGCGAAGA |
| | | | GTCAGTACGCATTAGCCGTGCAGCTCCT |
| | | | GCCGCAAGTTTCCCTGTGATGCAACAAG |
| | | | CTTACGCTGCACCAATGATGCAGCAGCC |
| | | | AGCTCAATCTAACGCAGCCGCTCCGGCG |
| | | | ACCGTTCCTTCCATGGAAGCGCCAGCAG |
| | | | CAGCGGAAATCAGTGGTCACATCGTACG |
| | | | TTCCCCGATGGTTGGTACTTTCTACCGC |
| | | | ACCCCAAGCCCGGACGCAAAAGCGTTCA |
| | | | TCGAAGTGGGTCAGAAAGTCAACGTGGG |
| | | | CGATACCCTGTGCATCGTTGAAGCCATG |
| | | | AAAATGATGAACCAGATCGAAGCGGACA |
| | | | AATCCGGTACCGTGAAAGCAATTCTGGT |
| | | | CGAAAGTGGACAACCGGTAGAATTTGAC |
| | | | GAGCCGCTGGTCGTCATCGAGTAACGAG |
| | | | GCGAACATGCTGGATAAAATTGTTATTG |
| | | | CCAACCGCGGCGAGATTGCATTGCGTAT |
| | | | TCTTCGTGCCTGTAAAGAACTGGGCATC |
| | | | AAGACTGTCGCTGTGCACTCCAGCGCGG |
| | | | ATCGCGATCTAAAACACGTATTACTGGC |
| | | | AGATGAAACGGTCTGTATTGGCCCTGCT |
| | | | CCGTCAGTAAAAAGTTATCTGAACATCC |
| | | | CGGCAATCATCAGCGCCGCTGAAATCAC |
| | | | CGGCGCAGTAGCAATCCATCCGGGTTAC |
| | | | GGCTTCCTCTCCGAGAACGCCAACTTTG |
| | | | CCGAGCAGGTTGAACGCTCCGGCTTTAT |
| | | | CTTCATTGGCCCGAAAGCAGAAACCATT |
| | | | CGCCTGATGGGCGACAAAGTATCCGCAA |
| | | | TCGCGGCGATGAAAAAAGCGGGCGTCCC |
| | | | TTGCGTACCGGGTTCTGACGGCCCGCTG |
| | | | GGCGACGATATGGATAAAAACCGTGCCA |
| | | | TTGCTAAACGCATTGGTTATCCGGTGAT |
| | | | TATCAAAGCCTCCGGCGGCGGCGGCGGT |
| | | | CGCGGTATGCGCGTAGTGCGCGGCGACG |
| | | | CTGAACTGGCACAATCCATCTCCATGAC |
| | | | CCGTGCGGAAGCGAAAGCTGCTTTCAGC |
| | | | AACGATATGGTTTACATGGAGAAATACC |
| | | | TGGAAAATCCTCGCCACGTCGAGATTCA |
| | | | GGTACTGGCTGACGGTCAGGGCAACGCT |
| | | | ATCTATCTGGCGGAACGTGACTGCTCCA |
| | | | TGCAACGCCGCCACCAGAAAGTGGTCGA |
| | | | AGAAGCGCCAGCACCGGGCATTACCCCG |
| | | | GAACTGCGTCGCTACATCGGCGAACGTT |
| | | | GCGCTAAAGCGTGTGTTGATATCGGCTA |
| | | | TCGCGGTGCAGGTACTTTCGAGTTCCTG |
| | | | TTCGAAAACGGCGAGTTCTATTTCATCG |
| | | | AAATGAACACCCGTATTCAGGTAGAACA |
| | | | CCCGGTTACAGAAATGATCACCGGCGTT |
| | | | GACCTGATCAAAGAACAGCTGCGTATCG |
| | | | CTGCCGGTCAACCGCTGTCGATCAAGCA |
| | | | AGAAGAAGTTCACGTTCGCGGCCATGCG |
| | | | GTGGAATGTCGTATCAACGCCGAAGATC |
| | | | CGAACACCTTCCTGCCAAGTCCGGGCAA |
| | | | AATCACCCGTTTCCACGCACCTGGCGGT |
| | | | TTTGGCGTACGTTGGGAGTCTCATATCT |
| | | | ACGCGGGCTACACCGTACCGCCGTACTA |
| | | | TGACTCAATGATCGGTAAGCTGATTTGC |
| | | | TACGGTGAAAACCGTGACGTGGCGATTG |
| | | | CCCGCATGAAGAATGCGCTGCAGGAGCT |
| | | | GATCATCGACGGTATCAAAACCAACGTT |
| | | | GATCTGCAGATCCGCATCATGAATGACG |
| | | | AGAACTTCCAGCATGGTGGCACTAACAT |
| | | | CCACTATCTGGAGAAAAAACTCGGTCTT |
| | | | CAGGAAAAATAAGACTGCTAAAGCGTCA |
| | | | AAAGGCCGGATTTTCCGGCCTTTTTTAT |
| | | | TACTGGGGATCGACAACCCCCATAAGGT |
| | | | ACAATCCCCGCTTTCTTCACCCATCAGG |
| | | | GACGCTCGGTCGCCTTTCACATTCCGCG |
| | | | AAAATTCATACCGTCGAGTTACGCCCGT |
| | | | TCTGCTTGACCTGGTAAAGTTACAACCA |
| | | | ATTAACCAATTCTGATTAGAAAAACTCA |
| | | | TCGAGCATCAAATGAAACTGCAATTTAT |
| | | | TCATATCAGGATTATCAATACCATATTT |
| | | | TTGAAAAAGCCGTTTCTGTAATGAAGGA |
| | | | GAAAACTCACCGAGGCAGTTCCATAGGA |
| | | | TGGCAAGATCCTGGTATCGGTCTGCGAT |
| | | | TCCGACTCGTCCAACATCAATACAACCT |
| | | | ATTAATTTCCCCTCGTCAAAAATAAGGT |
| | | | TATCAAGTGAGAAATCACCATGAGTGAC |
| | | | GACTGAATCCGGTGAGAATGGCAAAAGC |
| | | | TTATGCATTTCTTTCCAGACTTGTTCAA |
| | | | CAGGCCAGCCATTACGCTCGTCATCAAA |
| | | | ATCACTCGCATCAACCAAACCGTTATTC |
| | | | ATTCGTGATTGCGCCTGAGCGAGACGAA |
| | | | ATACGCGATCGCTGTTAAAAGGACAATT |
| | | | ACAAACAGGAATCGAATGCAACCGGCGC |
| | | | AGGAACACTGCCAGCGCATCAACAATAT |
| | | | TTTCACCTGAATCAGGATATTCTTCTAA |
| | | | TACCTGGAATGCTGTTTTCCCGGGGATC |
| | | | GCAGTGGTGAGTAACCATGCATCATCAG |
| | | | GAGTACGGATAAAATGCTTGATGGTCGG |
| | | | AAGAGGCATAAATTCCGTCAGCCAGTTT |
| | | | AGTCTGACCATCTCATCTGTAACATCAT |
| | | | TGGCAACGCTACCTTTGCCATGTTTCAG |
| | | | AAACAACTCTGGCGCATCGGGCTTCCCA |
| | | | TACAATCGATAGATTGTCGCACCTGATT |
| | | | GCCCGACATTATCGCGAGCCCATTTATA |
| | | | CCCATATAAATCAGCATCCATGTTGGAA |
| | | | TTTAATCGCGGCCTCGAGCAAGACGTTT |
| | | | CCCGTTGAATATGGCTCATAACACCCCT |
| | | | TGTATTACTGTTTATGTAAGCAGACAGT |
| | | | TTTATTGTTCATGATGATATATTTTTAT |
| | | | CTTGTGCAATGTAACATCAGAGATTTTG |
| | | | AGACACAACGTGGCTTTGTTGAATAAAT |
| | | | CGAACTTTTGCTGAGTTGAAGGATCAGA |
| | | | TCACGCATCTTCCCGACAACGCAGACCG |
| | | | TTCCGTGGCAAAGCAAAAGTTCAAAATC |
| | | | ACCAACTGGTCCACCTACAACAAAGCTC |
| | | | TCATCAACCGTGGCTCCCTCACTTTCTG |
| | | | GCTGGATGATGGGGCGATTCAGGCCTGG |
| | | | TATGAGTCAGCAACACCTTCTTCACGAG |
| | | | GCAGACCTCAGCGCTAGCGGAGTGTATA |
| | | | CTGGCTTACTATGTTGGCACTGATGAGG |
| | | | GTGTCAGTGAAGTGCTTCATGTGGCAGG |
| | | | AGAAAAAAGGCTGCACCGGTGCGTCAGC |
| | | | AGAATATGTGATACAGGATATATTCCGC |
| | | | TTCCTCGCTCACTGACTCGCTACGCTCG |
| | | | GTCGTTCGACTGCGGCGAGCGGAAATGG |
| | | | CTTACGAACGGGGCGGAGATTTCCTGGA |
| | | | AGATGCCAGGAAGATACTTAACAGGGAA |
| | | | GTGAGAGGGCCGCGGCAAAGCCGTTTTT |
| | | | CCATAGGCTCCGCCCCCCTGACAAGCAT |
| | | | CACGAAATCTGACGCTCAAATCAGTGGT |
| | | | GGCGAAACCCGACAGGACTATAAAGATA |
| | | | CCAGGCGTTTCCCCCTGGCGGCTCCCTC |
| | | | GTGCGCTCTCCTGTTCCTGCCTTTCGGT |
| | | | TTACCGGTGTCATTCCGCTGTTATGGCC |
| | | | GCGTTTGTCTCATTCCACGCCTGACACT |
| | | | CAGTTCCGGGTAGGCAGTTCGCTCCAAG |
| | | | CTGGACTGTATGCACGAACCCCCCGTTC |
| | | | AGTCCGACCGCTGCGCCTTATCCGGTAA |
| | | | CTATCGTCTTGAGTCCAACCCGGAAAGA |
| | | | CATGCAAAAGCACCACTGGCAGCAGCCA |
| | | | CTGGTAATTGATTTAGAGGAGTTAGTCT |
| | | | TGAAGTCATGCGCCGGTTAAGGCTAAAC |
| | | | TGAAAGGACAAGTTTTGGTGACTGCGCT |
| | | | CCTCCAAGCCAGTTACCTCGGTTCAAAG |
| | | | AGTTGGTAGCTCAGAGAACCTTCGAAAA |
| | | | ACCGCCCTGCAAGGCGGTTTTTTCGTTT |
| | | | TCAGAGCAAGAGATTACGCGCAGACCAA |
| | | | AACGATCTCAAGAAGATCATCTTATTAA |
| | | | GGGGTCTGACGCTCAGTGGAACGAAAAC |
| | | | TCACGTTAAGGGATTTTGGTCATGAGAT |
| | | | TATCAAAAAGGATCTTCACCTAGATCCT |
| | | | TTTAAATTAAAAATGAAGTTTTAAATCA |
| | | | ATCTAAAGTATATATGAGTAAACTTGGT |
| | | | CTGACAGGTGAGCTGATACCGCTCGCCG |
| | | | CATGCACATGCAGTCATGTCGTGC |
| 35. | | pNH243 | ACCAGCAAATCGCGCTGTTAGCGGGCCC |
| | | | ATTAAGTTCTGTCTCGGCGCGTCTGCGT |
| | | | CTGGCTGGCTGGCATAAATATCTCACTC |
| | | | GCAATCAAATTCAGCCGATAGCGGAACG |
| | | | GGAAGGCGACTGGAGTGCCATGTCCGGT |
| | | | TTTCAACAAACCATGCAAATGCTGAATG |
| | | | AGGGCATCGTTCCCACTGCGATGCTGGT |
| | | | TGCCAACGATCAGATGGCGCTGGGCGCA |
| | | | ATGCGCGCCATTACCGAGTCCGGGCTGC |
| | | | GCGTTGGTGCGGATATTTCGGTAGTGGG |
| | | | ATACGACGATACCGAAGACAGCTCATGT |
| | | | TATATCCCGCCGTTAACCACCATCAAAC |
| | | | AGGATTTTCGCCTGCTGGGGCAAACCAG |
| | | | CGTGGACCGCTTGCTGCAACTCTCTCAG |
| | | | GGCCAGGCGGTGAAGGGCAATCAGCTGT |
| | | | TGCCCGTCTCACTGGTGAAAAGAAAAAC |
| | | | CACCCTGGCGCCCAATACGCAAACCGCC |
| | | | TCTCCCCGCGCGTTGGCCGATTCATTAA |
| | | | TGCAGCTGGCACGACAGGTTTCCCGACT |
| | | | GGAAAGCGGGCAGTGAGCGCAACGCAAT |
| | | | TAATGTAAGTTAGCTCACTCATTAGGCA |
| | | | CAATTCTCATGTTTGACAGCTTATCATC |
| | | | GACTGCACGGTGCACCAATGCTTCTGGC |
| | | | GTCAGGCAGCCATCGGAAGCTGTGGTAT |
| | | | GGCTGTGCAGGTCGTAAATCACTGCATA |
| | | | ATTCGTGTCGCTCAAGGCGCACTCCCGT |
| | | | TCTGGATAATGTTTTTTGCGCCGACATC |
| | | | ATAACGGTTCTGGCAAATATTCTGAAAT |
| | | | GAGCTGTTGACAATTAATCATCGGCTCG |
| | | | TATAATGTGTGGAATTGTGAGCGGATAA |
| | | | CAATTTCACACAGGAAACAGCCAGTCCG |
| | | | TTTAGGTGTTTTCACGAGCAATTGACCA |
| | | | ACAAGGACAGGAGGTATTAATGTCGGCG |
| | | | ACGACGGGCGCACGTAGCGCCTCTGTTG |
| | | | GATGGGCAGAATCACTGATTGGGTTGCA |
| | | | TTTGGGCAAGGTCGCCCTGATTACGGGT |
| | | | GGATCTGCCGGCATTGGTGGGCAGATTG |
| | | | GTCGCTTATTGGCTTTATCTGGTGCACG |
| | | | TGTGATGCTGGCGGCACGTGATCGCCAC |
| | | | AAATTGGAACAGATGCAAGCTATGATTC |
| | | | AGAGTGAATTAGCGGAAGTTGGCTACAC |
| | | | AGATGTGGAGGATCGCGTTCATATCGCA |
| | | | CCAGGGTGCGACGTTTCAAGTGAGGCCC |
| | | | AACTTGCAGACTTGGTTGAACGCACATT |
| | | | GTCAGCTTTCGGTACCGTTGACTATTTA |
| | | | ATCAATAACGCCGGCATTGCGGGTGTAG |
| | | | AGGAGATGGTTATCGACATGCCCGTGGA |
| | | | GGGTTGGCGTCATACGCTGTTCGCAAAC |
| | | | CTCATCTCGAACTACAGCCTTATGCGTA |
| | | | AGCTGGCACCACTGATGAAGAAGCAGGG |
| | | | GTCGGGGTACATCCTCAACGTAAGCTCG |
| | | | TATTTCGGAGGGGAGAAAGACGCAGCAA |
| | | | TTCCGTATCCCAACCGTGCCGACTATGC |
| | | | TGTTAGTAAAGCCGGCCAGCGCGCTATG |
| | | | GCTGAAGTCTTTGCGCGCTTTTTGGGAC |
| | | | CCGAGATTCAGATCAATGCTATTGCACC |
| | | | TGGACCCGTAGAGGGAGATCGTCTGCGC |
| | | | GGAACTGGTGAGCGTCCTGGATTGTTCG |
| | | | CTCGCCGTGCACGCCTTATCTTGGAGAA |
| | | | CAAGCGTTTAAATGAGCTTCATGCTGCG |
| | | | TTAATCGCGGCAGCTCGTACCGATGAAC |
| | | | GTTCGATGCATGAGTTGGTTGAATTGTT |
| | | | GTTGCCGAATGACGTCGCGGCGCTTGAA |
| | | | CAAAACCCCGCTGCCCCTACCGCACTTC |
| | | | GTGAGCTGGCGCGTCGCTTCCGCAGTGA |
| | | | GGGAGACCCTGCAGCGTCTTCGTCCAGT |
| | | | GCTTTATTAAATCGCTCCATTGCGGCGA |
| | | | AATTACTTGCTCGTTTGCACAATGGTGG |
| | | | ATATGTTCTGCCAGCAGACATTTTTGCC |
| | | | AATTTGCCGAACCCACCAGACCCTTTTT |
| | | | TCACCCGCGCCCAGATCGACCGCGAGGC |
| | | | TCGTAAGGTACGCGATGGAATTATGGGA |
| | | | ATGCTTTATCTTCAGCGCATGCCTACGG |
| | | | AATTTGATGTTGCGATGGCGACGGTTTA |
| | | | TTATTTGGCGGACCGTGTTGTCTCAGGC |
| | | | GAGACTTTCCATCCGTCTGGAGGTTTGC |
| | | | GCTACGAACGCACCCCGACAGGGGGAGA |
| | | | ATTGTTTGGCCTGCCTTCGCCGGAACGT |
| | | | TTAGCAGAGCTTGTCGGCTCCACAGTCT |
| | | | ATCTGATCGGTGAACATTTAACTGAGCA |
| | | | TTTAAACTTGCTTGCACGCGCTTACTTA |
| | | | GAGCGCTACGGGGCACGCCAGGTAGTTA |
| | | | TGATCGTAGAAACGGAAACTGGAGCTGA |
| | | | AACCATGCGTCGTTTACTGCACGACCAC |
| | | | GTAGAGGCAGGACGCTTGATGACGATTG |
| | | | TGGCTGGTGACCAGATCGAGGCCGCGAT |
| | | | TGACCAAGCGATTACTCGTTACGGACGT |
| | | | CCAGGTCCCGTAGTCTGTACCCCTTTTC |
| | | | GTCCATTGCCCACTGTTCCTCTTGTAGG |
| | | | CCGCAAGGACAGCGATTGGTCTACCGTC |
| | | | TTGAGCGAGGCAGAATTCGCTGAGTTAT |
| | | | GTGAGCATCAGTTAACGCATCACTTCCG |
| | | | TGTAGCACGTTGGATCGCCCTGTCTGAC |
| | | | GGTGCACGTCTTGCCTTAGTCACGCCCG |
| | | | AGACTACGGCAACTAGCACAACCGAGCA |
| | | | GTTCGCCTTGGCCAACTTCATTAAGACA |
| | | | ACGCTTCATGCTTTCACCGCAACGATCG |
| | | | GTGTAGAGTCTGAACGCACAGCGCAGCG |
| | | | CATCCTGATCAATCAAGTCGATTTAACT |
| | | | CGTCGTGCTCGCGCAGAGGAGCCGCGTG |
| | | | ACCCTCACGAACGTCAACAGGAATTGGA |
| | | | GCGCTTCATCGAAGCCGTGCTGTTAGTT |
| | | | ACCGCACCGTTGCCACCCGAAGCGGACA |
| | | | CTCGTTATGCCGGACGTATCCACCGCGG |
| | | | TCGCGCGATTACGGTATAATTAAGAAAG |
| | | | GAGGTACTCAATGTCAGGAACAGGGCGC |
| | | | TTGGCGGGAAAAATTGCCTTGATTACGG |
| | | | GAGGTGCCGGCAATATTGGCTCCGAATT |
| | | | AACCCGTCGCTTCCTGGCAGAGGGTGCG |
| | | | ACAGTCATTATCTCTGGACGTAATCGTG |
| | | | CTAAGCTGACTGCGCTGGCCGAGCGTAT |
| | | | GCAAGCTGAGGCCGGCGTCCCCGCTAAA |
| | | | CGCATTGACTTGGAAGTTATGGACGGAA |
| | | | GCGACCCAGTTGCCGTGCGCGCAGGCAT |
| | | | TGAGGCTATTGTTGCTCGCCACGGCCAG |
| | | | ATTGACATTCTTGTCAACAACGCCGGTA |
| | | | GCGCTGGTGCTCAGCGTCGTTTAGCGGA |
| | | | AATCCCTTTAACTGAAGCAGAATTGGGT |
| | | | CCCGGAGCAGAGGAAACATTGCACGCTA |
| | | | GTATCGCCAATCTTCTTGGAATGGGCTG |
| | | | GCATCTGATGCGCATTGCGGCCCCGCAC |
| | | | ATGCCCGTTGGATCAGCTGTTATCAATG |
| | | | TGTCCACCATCTTTTCTCGTGCCGAATA |
| | | | CTACGGACGTATCCCATATGTGACCCCC |
| | | | AAAGCCGCACTGAATGCGTTAAGCCAGC |
| | | | TGGCTGCGCGCGAACTGGGCGCGCGTGG |
| | | | AATTCGCGTAAATACAATCTTTCCGGGT |
| | | | CCCATCGAATCGGACCGCATTCGCACTG |
| | | | TATTTCAACGTATGGATCAGCTGAAGGG |
| | | | CCGTCCTGAGGGTGACACGGCGCACCAT |
| | | | TTCTTGAACACGATGCGCCTGTGTCGCG |
| | | | CAAACGACCAAGGGGCATTAGAACGCCG |
| | | | CTTCCCTTCCGTCGGCGATGTCGCCGAT |
| | | | GCCGCGGTCTTCTTGGCTTCTGCTGAGT |
| | | | CTGCAGCATTATCTGGGGAAACGATTGA |
| | | | GGTGACCCATGGAATGGAGCTGCCGGCC |
| | | | TGTTCGGAAACGAGTCTGTTAGCGCGCA |
| | | | CTGATCTGCGCACTATCGATGCCTCTGG |
| | | | CCGTACCACCCTTATCTGTGCCGGGGAT |
| | | | CAGATTGAGGAGGTAATGGCTTTAACGG |
| | | | GTATGCTGCGCACATGCGGATCGGAGGT |
| | | | AATTATTGGTTTTCGTAGCGCCGCCGCA |
| | | | TTAGCACAGTTTGAACAGGCAGTAAATG |
| | | | AGTCGCGTCGTTTAGCTGGCGCAGACTT |
| | | | TACCCCGCCGATCGCCTTACCTCTTGAT |
| | | | CCGCGTGACCCTGCTACGATCGACGCTG |
| | | | TATTCGATTGGGGAGCTGGAGAAAACAC |
| | | | TGGGGGTATTCATGCGGCGGTTATCCTG |
| | | | CCCGCGACATCCCATGAGCCCGCACCGT |
| | | | GTGTAATTGAAGTAGATGACGAACGTGT |
| | | | GCTGAACTTTCTTGCGGACGAAATCACC |
| | | | GGGACGATTGTCATTGCTAGCCGTCTTG |
| | | | CTCGCTACTGGCAGTCACAACGCCTGAC |
| | | | GCCTGGGGCGCGCGCCCGCGGACCACGT |
| | | | GTGATCTTTCTGTCTAATGGGGCAGATC |
| | | | AGAACGGAAATGTTTATGGCCGCATTCA |
| | | | GTCGGCAGCCATCGGTCAATTAATTCGT |
| | | | GTTTGGCGCCACGAAGCCGAGCTGGACT |
| | | | ATCAACGCGCGTCTGCCGCGGGCGACCA |
| | | | TGTTCTGCCGCCCGTATGGGCAAACCAA |
| | | | ATCGTACGTTTTGCAAACCGCTCGCTGG |
| | | | AGGGTTTGGAGTTCGCCTGCGCATGGAC |
| | | | CGCTCAACTTTTACACTCCCAACGCCAT |
| | | | ATTAACGAAATCACCCTGAACATCCCAG |
| | | | CCAATATCTAAATGACTTAGGAGCGCTC |
| | | | TCCTGAGTAGGACAAATCCGCCGGGAGC |
| | | | GGATTTGAACGTTGCGAAGCAACGGCCC |
| | | | GGAGGGTGGCGGGCAGGACGCCCGCCAT |
| | | | AAACTGCCAGGCATCAAATTAAGCAGAA |
| | | | GGCCATCCTGACGGATGGCCTTTTTGCG |
| | | | TTTCTACAAACTCTTTCGGTCCGTTGTT |
| | | | TATTTTTCTAAATACATTCAAATATGTA |
| | | | TCCGCTCATGAGACAATAACCCTGATAA |
| | | | ATGCTTCAATAATATTGAAAAAGGAAGA |
| | | | GTATGAGTATTCAACATTTCCGTGTCGC |
| | | | CCTTATTCCCTTTTTTGCGGCATTTTGC |
| | | | CTTCCTGTTTTTGCTCACCCAGAAACGC |
| | | | TGGTGAAAGTAAAAGATGCTGAAGATCA |
| | | | GTTGGGTGCACGAGTGGGTTACATCGAA |
| | | | CTGGATCTCAACAGCGGTAAGATCCTTG |
| | | | AGAGTTTTCGCCCCGAAGAACGTTTCCC |
| | | | AATGATGAGCACTTTTAAAGTTCTGCTA |
| | | | TGTGGCGCGGTATTATCCCGTGTTGACG |
| | | | CCGGGCAAGAGCAACTCGGTCGCCGCAT |
| | | | ACACTATTCTCAGAATGACTTGGTTGAG |
| | | | TACTCACCAGTCACAGAAAAGCATCTTA |
| | | | CGGATGGCATGACAGTAAGAGAATTATG |
| | | | CAGTGCTGCCATAACCATGAGTGATAAC |
| | | | ACTGCGGCCAACTTACTTCTGACAACGA |
| | | | TCGGAGGACCGAAGGAGCTAACCGCTTT |
| | | | TTTGCACAACATGGGGGATCATGTAACT |
| | | | CGCCTTGATCGTTGGGAACCGGAGCTGA |
| | | | ATGAAGCCATACCAAACGACGAGCGTGA |
| | | | CACCACGATGCCTGTAGCAATGGCAACA |
| | | | ACGTTGCGCAAACTATTAACTGGCGAAC |
| | | | TACTTACTCTAGCTTCCCGGCAACAATT |
| | | | AATAGACTGGATGGAGGCGGATAAAGTT |
| | | | GCAGGACCACTTCTGCGCTCGGCCCTTC |
| | | | CGGCTGGCTGGTTTATTGCTGATAAATC |
| | | | TGGAGCCGGTGAGCGTGGGTCTCGCGGT |
| | | | ATCATTGCAGCACTGGGGCCAGATGGTA |
| | | | AGCCCTCCCGTATCGTAGTTATCTACAC |
| | | | GACGGGGAGTCAGGCAACTATGGATGAA |
| | | | CGAAATAGACAGATCGCTGAGATAGGTG |
| | | | CCTCACTGATTAAGCATTGGTAACTGTC |
| | | | AGACCAAGTTTACTCATATATACTTTAG |
| | | | ATTGATTTCCTTAGGACTGAGCGTCAAC |
| | | | CCCGTAGAAAAGATCAAAGGATCTTCTT |
| | | | GAGATCCTTTTTTTCTGCGCGTAATCTG |
| | | | CTGCTTGCAAACAAAAAAACCACCGCTA |
| | | | CCAGCGGTGGTTTGTTTGCCGGATCAAG |
| | | | AGCTACCAACTCTTTTTCCGAAGGTAAC |
| | | | TGGCTTCAGCAGAGCGCAGATACCAAAT |
| | | | ACTGTCCTTCTAGTGTAGCCGTAGTTAG |
| | | | GCCACCACTTCAAGAACTCTGTAGCACC |
| | | | GCCTACATACCTCGCTCTGCTAATCCTG |
| | | | TTACCAGTGGCTGCTGCCAGTGGCGATA |
| | | | AGTCGTGTCTTACCGGGTTGGACTCAAG |
| | | | ACGATAGTTACCGGATAAGGCGCAGCGG |
| | | | TCGGGCTGAACGGGGGGTTCGTGCACAC |
| | | | AGCCCAGCTTGGAGCGAACGACCTACAC |
| | | | CGAACTGAGATACCTACAGCGTGAGCTA |
| | | | TGAGAAAGCGCCACGCTTCCCGAAGGGA |
| | | | GAAAGGCGGACAGGTATCCGGTAAGCGG |
| | | | CAGGGTCGGAACAGGAGAGCGCACGAGG |
| | | | GAGCTTCCAGGGGGAAACGCCTGGTATC |
| | | | TTTATAGTCCTGTCGGGTTTCGCCACCT |
| | | | CTGACTTGAGCGTCGATTTTTGTGATGC |
| | | | TCGTCAGGGGGGCGGAGCCTATGGAAAA |
| | | | ACGCCAGCAACGCGGCCTTTTTACGGTT |
| | | | CCTGGCCTTTTGCTGGCCTTTTGCTCAC |
| | | | ATGTTCTTTCCTGCGTTATCCCCTGATT |
| | | | CTGTGGATAACCGTATTACCGCCTTTGA |
| | | | GTGAGCTGATACCGCTCGCCGCAGCCGA |
| | | | ACGACCGAGCGCAGCGAGTCAGTGAGCG |
| | | | AGGAAGCGGAAGAGCGCCTGATGCGGTA |
| | | | TTTTCTCCTTACGCATCTGTGCGGTATT |
| | | | TCACACCGCATATAAGGTGCACTGTGAC |
| | | | TGGGTCATGGCTGCGCCCCGACACCCGC |
| | | | CAACACCCGCTGACGCGCCCTGACGGGC |
| | | | TTGTCTGCTCCCGGCATCCGCTTACAGA |
| | | | CAAGCTGTGACCGTCTCCGGGAGCTGCA |
| | | | TGTGTCAGAGGTTTTCACCGTCATCACC |
| | | | GAAACGCGCGAGGCAGCTGCGGTAAAGC |
| | | | TCATCAGCGTGGTCGTGCAGCGATTCAC |
| | | | AGATGTCTGCCTGTTCATCCGCGTCCAG |
| | | | CTCGTTGAGTTTCTCCAGAAGCGTTAAT |
| | | | GTCTGGCTTCTGATAAAGCGGGCCATGT |
| | | | TAAGGGCGGTTTTTTCCTGTTTGGTCAC |
| | | | TGATGCCTCCGTGTAAGGGGGATTTCTG |
| | | | TTCATGGGGGTAATGATACCGATGAAAC |
| | | | GAGAGAGGATGCTCACGATACGGGTTAC |
| | | | TGATGATGAACATGCCCGGTTACTGGAA |
| | | | CGTTGTGAGGGTAAACAACTGGCGGTAT |
| | | | GGATGCGGCGGGACCAGAGAAAAATCAC |
| | | | TCAGGGTCAATGCCAGCGCTTCGTTAAT |
| | | | ACAGATGTAGGTGTTCCACAGGGTAGCC |
| | | | AGCAGCATCCTGCGATGCAGATCCGGAA |
| | | | CATAATGGTGCAGGGCGCTGACTTCCGC |
| | | | GTTTCCAGACTTTACGAAACACGGAAAC |
| | | | CGAAGACCATTCATGTTGTTGCTCAGGT |
| | | | CGCAGACGTTTTGCAGCAGCAGTCGCTT |
| | | | CACGTTCGCTCGCGTATCGGTGATTCAT |
| | | | TCTGCTAACCAGTAAGGCAACCCCGCCA |
| | | | GCCTAGCCGGGTCCTCAACGACAGGAGC |
| | | | ACGATCATGCGCACCCGTGGCCAGGACC |
| | | | CAACGCTGCCCGAAATTCCGACACCATC |
| | | | GAATGGTGCAAAACCTTTCGCGGTATGG |
| | | | CATGATAGCGCCCGGAAGAGAGTCAATT |
| | | | CAGGGTGGTGAATGTGAAACCAGTAACG |
| | | | TTATACGATGTCGCAGAGTATGCCGGTG |
| | | | TCTCTTATCAGACCGTTTCCCGCGTGGT |
| | | | GAACCAGGCCAGCCACGTTTCTGCGAAA |
| | | | ACGCGGGAAAAAGTGGAAGCGGCGATGG |
| | | | CGGAGCTGAATTACATTCCCAACCGCGT |
| | | | GGCACAACAACTGGCGGGCAAACAGTCG |
| | | | TTGCTGATTGGCGTTGCCACCTCCAGTC |
| | | | TGGCCCTGCACGCGCCGTCGCAAATTGT |
| | | | CGCGGCGATTAAATCTCGCGCCGATCAA |
| | | | CTGGGTGCCAGCGTGGTGGTGTCGATGG |
| | | | TAGAACGAAGCGGCGTCGAAGCCTGTAA |
| | | | AGCGGCGGTGCACAATCTTCTCGCGCAA |
| | | | CGCGTCAGTGGGCTGATCATTAACTATC |
| | | | CGCTGGATGACCAGGATGCCATTGCTGT |
| | | | GGAAGCTGCCTGCACTAATGTTCCGGCG |
| | | | TTATTTCTTGATGTCTCTGACCAGACAC |
| | | | CCATCAACAGTATTATTTTCTCCCATGA |
| | | | AGACGGTACGCGACTGGGCGTGGAGCAT |
| | | | CTGGTCGCATTGGGTC |
| 36. | | pNH265 | TTGTTTCATCAAGCCTTACGGTCACCGT |
| | | | AACCAGCAAATCAATATCACTGTGTGGC |
| | | | TTCAGGCCGCCATCCACTGCGGAGCCGT |
| | | | ACAAATGTACGGCCAGCAACGTCGGTTC |
| | | | GAGATGGCGCTCGATGACGCCAACTACC |
| | | | TCTGATAGTTGAGTCGATACTTCGGCGA |
| | | | TCACCGCTTCCCTCATACTCTTCCTTTT |
| | | | TCAATATTATTGAAGCATTTATCAGGGT |
| | | | TATTGTCTCATGAGCGGATACATATTTG |
| | | | AATGTATTTAGAAAAATAAACAAATAGC |
| | | | TAGCTCACTCGGTCGCTACGCTCCGGGC |
| | | | GTGAGACTGCGGCGGGCGCTGCGGACAC |
| | | | ATACAAAGTTACCCACAGATTCCGTGGA |
| | | | TAAGCAGGGGACTAACATGTGAGGCAAA |
| | | | ACAGCAGGGCCGCGCCGGTGGCGTTTTT |
| | | | CCATAGGCTCCGCCCTCCTGCCAGAGTT |
| | | | CACATAAACAGACGCTTTTCCGGTGCAT |
| | | | CTGTGGGAGCCGTGAGGCTCAACCATGA |
| | | | ATCTGACAGTACGGGCGAAACCCGACAG |
| | | | GACTTAAAGATCCCCACCGTTTCCGGCT |
| | | | GGTCGCTCCCTCTTGCGCTCTCCTGTTC |
| | | | CGACCCTGCCGTTTACCGGATACCTGTT |
| | | | CCGCCTTTCTCCCTTACGGGAAGTGTGG |
| | | | CGCTTTCTCATAGCTCACACACTGGTAT |
| | | | CTCGGCTCGGTGTAGGTCGTTCGCTCCA |
| | | | AGCTGGGCTGTAAGCAAGAACTCCCCGT |
| | | | TCAGCCCGACTGCTGCGCCTTATCCGGT |
| | | | AACTGTTCACTTGAGTCCAACCCGGAAA |
| | | | AGCACGGTAAAACGCCACTGGCAGCAGC |
| | | | CATTGGTAACTGGGAGTTCGCAGAGGAT |
| | | | TTGTTTAGCTAAACACGCGGTTGCTCTT |
| | | | GAAGTGTGCGCCAAAGTCCGGCTACACT |
| | | | GGAAGGACAGATTTGGTTGCTGTGCTCT |
| | | | GCGAAAGCCAGTTACCACGGTTAAGCAG |
| | | | TTCCCCAACTGACTTAACCTTCGATCAA |
| | | | ACCACCTCCCAATGTGGTTTTTTCGTTT |
| | | | ACAGGGCAAAAGATTACGCGCAGAAAAA |
| | | | AAGGATCTCAAGAAGATCCTTTGATCTT |
| | | | TTCTACTGAACCGCTCTAGATTTCAGTG |
| | | | CAATTTATCTCTTCAAATGTAGCACCTG |
| | | | AAGTCAGCCCCATACGATATAAGTTGTA |
| | | | ATTCTCATGTTAGTCATGCCCCGCGCCC |
| | | | ACCGGAAGGAGCTGACTGGGTTGAAGGC |
| | | | TCTCAAGGGCATCGGTCGAGATCCCGGT |
| | | | GCCTAATGAGTGAGCTAACTTTTGACGG |
| | | | CTAGCTCAGTCCTAGGGATAATGCTAGC |
| | | | ACCAGCCTCGAGGGAAACCACGTAAGCT |
| | | | CCGGCGTTTAAACACCCATAACAGATAC |
| | | | GGACTTTCTCAAAGGAGAGTTATCAGTG |
| | | | AAAATCCGCCCGTTACATGACCGTGTCA |
| | | | TCATCAAACGCTTGGAAGAAGAGCGTAC |
| | | | CTCGGCGGGCGGGATTGTCATTCCAGAT |
| | | | AGCGCAGCTGAAAAACCGATGCGTGGTG |
| | | | AAATCCTGGCAGTGGGCAATGGAAAAGT |
| | | | GCTTGATAATGGAGAGGTACGTGCTTTA |
| | | | CAGGTGAAAGTGGGTGATAAAGTGCTCT |
| | | | TTGGGAAATACGCGGGTACGGAGGTTAA |
| | | | AGTAGATGGGGAAGATGTTGTTGTCATG |
| | | | CGTGAAGATGACATTCTGGCTGTGTTAG |
| | | | AATCTTAATCCGCGCACGACACTGAACA |
| | | | TACGAATTTAAGGAATAAAGATAATGGC |
| | | | GAAAGAAGTTGTGTATCGTGGTAGTGCG |
| | | | CGCCAGCGTATGATGCAGGGTATTGAAA |
| | | | TTCTCGCTCGCGCCGCTATTCCAACGCT |
| | | | GGGGGCAACCGGCCCGAGCGTCATGATT |
| | | | CAACATCGCGCCGATGGTCTGCCACCCA |
| | | | TTTCTACACGCGATGGCGTTACCGTAGC |
| | | | GAATTCTATTGTTTTAAAAGACCGTGTC |
| | | | GCGAACCTGGGTGCCCGCCTGCTGCGCG |
| | | | ACGTAGCCGGTACAATGAGCCGTGAAGC |
| | | | CGGCGACGGCACGACGACTGCGATCGTA |
| | | | TTGGCCCGCCACATCGCCCGTGAGATGT |
| | | | TTAAATCGCTGGCCGTGGGTGCAGATCC |
| | | | GATCGCGCTGAAACGTGGTATCGATCGC |
| | | | GCCGTTGCTCGTGTGTCCGAAGATATTG |
| | | | GGGCGCGTGCGTGGCGTGGCGATAAAGA |
| | | | AAGCGTGATCCTGGGTGTCGCTGCTGTG |
| | | | GCGACGAAAGGCGAACCGGGCGTTGGCC |
| | | | GTCTGCTGCTGGAGGCTCTCGATGCAGT |
| | | | GGGTGTTCACGGTGCCGTTTCTATCGAA |
| | | | CTGGGCCAACGTCGTGAAGATCTGCTGG |
| | | | ACGTCGTCGATGGCTATCGCTGGGAAAA |
| | | | AGGTTATTTATCTCCCTACTTTGTCACG |
| | | | GACCGTGCCCGCGAACTCGCGGAACTGG |
| | | | AGGATGTCTACCTGCTCATGACCGACCG |
| | | | CGAAGTGGTTGACTTCATCGACCTTGTA |
| | | | CCTCTGCTGGAGGCCGTGACGGAAGCAG |
| | | | GAGGCTCCCTGCTGATTGCCGCGGATCG |
| | | | TGTGCACGAAAAGGCCTTAGCGGGGCTG |
| | | | CTTCTGAATCACGTGCGCGGTGTCTTCA |
| | | | AGGCCGTGGCCGTAACCGCTCCGGGTTT |
| | | | TGGCGACAAACGCCCGAACCGTTTACTT |
| | | | GACCTGGCCGCGTTAACCGGCGGTCGTG |
| | | | CCGTGCTCGAAGCTCAAGGCGACCGTCT |
| | | | GGACCGTGTTACCCTCGCGGATCTGGGC |
| | | | CGTGTGCGCCGTGCCGTGGTGTCGGCAG |
| | | | ATGATACCGCGCTGCTTGGCATCCCGGG |
| | | | CACCGAAGCTAGCCGTGCACGCCTCGAA |
| | | | GGTCTGCGTTTAGAAGCAGAGCAGTACC |
| | | | GTGCGCTGAAACCAGGGCAGGGTTCTGC |
| | | | CACCGGGCGCCTGCACGAACTTGAAGAA |
| | | | ATTGAAGCGCGCATTGTGGGTCTGTCCG |
| | | | GAAAGAGCGCCGTTTATCGCGTCGGAGG |
| | | | TGTGACCGATGTGGAAATGAAAGAGCGC |
| | | | ATGGTTCGCATCGAAAACGCTTACCGTT |
| | | | CGGTGGTAAGTGCGCTGGAGGAAGGCGT |
| | | | GCTCCCTGGCGGTGGTGTCGGCTTTCTG |
| | | | GGTAGTATGCCGGTGCTTGCGGAATTGG |
| | | | AGGCCCGCGACGCAGATGAAGCTCGCGG |
| | | | GATTGGGATTGTACGCAGCGCCTTAACG |
| | | | GAGCCTCTTCGTATTATCGGCGAAAATA |
| | | | GTGGCTTGAGCGGTGAAGCCGTTGTTGC |
| | | | CAAAGTCATGGATCATGCCAACCCGGGA |
| | | | TGGGGTTACGACCAGGAGTCTGGCTCTT |
| | | | TTTGCGACCTGCATGCGCGTGGGATCTG |
| | | | GGATGCTGCTAAAGTGTTACGTCTCGCG |
| | | | TTGGAGAAGGCAGCCTCTGTTGCTGGGA |
| | | | CCTTTCTGACAACCGAAGCTGTTGTTCT |
| | | | CGAAATTCCGGATACAGATGCGTTCGCA |
| | | | GGGTTCAGTGCAGAATGGGCTGCCGCCA |
| | | | CGCGCGAAGATCCGCGCGTATGAGTTTA |
| | | | AACGCGGCCGCAATTTGAACGCACCCAT |
| | | | AACAGATACGGACTTTCTCAAAGGAGAG |
| | | | TTATCAATGAATATTCGTCCATTGCATG |
| | | | ATCGCGTGATCGTCAAGCGTAAAGAAGT |
| | | | TGAAACTAAATCTGCTGGCGGCATCGTT |
| | | | CTGACCGGCTCTGCAGCGGCTAAATCCA |
| | | | CCCGCGGCGAAGTGCTGGCTGTCGGCAA |
| | | | TGGCCGTATCCTTGAAAATGGCGAAGTG |
| | | | AAGCCGCTGGATGTGAAAGTTGGCGACA |
| | | | TCGTTATTTTCAACGATGGCTACGGTGT |
| | | | GAAATCTGAGAAGATCGACAATGAAGAA |
| | | | GTGTTGATCATGTCCGAAAGCGACATTC |
| | | | TGGCAATTGTTGAAGCGTAATCCGCGCA |
| | | | CGACACTGAACATACGAATTTAAGGAAT |
| | | | AAAGATAATGGCAGCTAAAGACGTAAAA |
| | | | TTCGGTAACGACGCTCGTGTGAAAATGC |
| | | | TGCGCGGCGTAAACGTACTGGCAGATGC |
| | | | AGTGAAAGTTACCCTCGGTCCAAAAGGC |
| | | | CGTAACGTAGTTCTGGATAAATCTTTCG |
| | | | GTGCACCGACCATCACCAAAGATGGTGT |
| | | | TTCCGTTGCTCGTGAAATCGAACTGGAA |
| | | | GACAAGTTCGAAAATATGGGTGCGCAGA |
| | | | TGGTGAAAGAAGTTGCCTCTAAAGCAAA |
| | | | CGACGCTGCAGGCGACGGTACCACCACT |
| | | | GCAACCGTACTGGCTCAGGCTATCATCA |
| | | | CTGAAGGTCTGAAAGCTGTTGCTGCGGG |
| | | | CATGAACCCGATGGACCTGAAACGTGGT |
| | | | ATCGACAAAGCGGTTACCGCTGCAGTTG |
| | | | AAGAACTGAAAGCGCTGTCCGTACCATG |
| | | | CTCTGACTCTAAAGCGATTGCTCAGGTT |
| | | | GGTACCATCTCCGCTAACTCCGACGAAA |
| | | | CCGTAGGTAAACTGATCGCTGAAGCGAT |
| | | | GGACAAAGTCGGTAAAGAAGGCGTTATC |
| | | | ACCGTTGAAGACGGTACCGGTCTGCAGG |
| | | | ACGAACTGGACGTGGTTGAAGGTATGCA |
| | | | GTTCGACCGTGGCTACCTGTCTCCTTAC |
| | | | TTCATCAACAAGCCGGAAACTGGCGCAG |
| | | | TAGAACTGGAAAGCCCGTTCATCCTGCT |
| | | | GGCTGACAAGAAAATCTCCAACATCCGC |
| | | | GAAATGCTGCCGGTTCTGGAAGCTGTTG |
| | | | CCAAAGCAGGCAAACCGCTGCTGATCAT |
| | | | CGCTGAAGATGTAGAAGGCGAAGCGCTG |
| | | | GCAACTCTGGTTGTTAACACCATGCGTG |
| | | | GCATCGTGAAAGTCGCTGCGGTTAAAGC |
| | | | ACCGGGCTTCGGCGATCGTCGTAAAGCT |
| | | | ATGCTGCAGGATATCGCAACCCTGACTG |
| | | | GCGGTACCGTGATCTCTGAAGAGATCGG |
| | | | TATGGAGCTGGAAAAAGCAACCCTGGAA |
| | | | GACCTGGGTCAGGCTAAACGTGTTGTGA |
| | | | TCAACAAAGACACCACCACTATCATCGA |
| | | | TGGCGTGGGTGAAGAAGCTGCAATCCAG |
| | | | GGCCGTGTTGCTCAGATCCGTCAGCAGA |
| | | | TTGAAGAAGCAACTTCTGACTACGACCG |
| | | | TGAAAAACTGCAGGAACGCGTAGCGAAA |
| | | | CTGGCAGGCGGCGTTGCAGTTATCAAAG |
| | | | TGGGTGCTGCTACCGAAGTTGAAATGAA |
| | | | AGAGAAAAAAGCACGCGTTGAAGATGCC |
| | | | CTGCACGCGACCCGTGCTGCGGTAGAAG |
| | | | AAGGCGTGGTTGCTGGTGGTGGTGTTGC |
| | | | GCTGATCCGCGTAGCGTCTAAACTGGCT |
| | | | GACCTGCGTGGTCAGAACGAAGACCAGA |
| | | | ACGTGGGTATCAAAGTTGCACTGCGTGC |
| | | | AATGGAAGCTCCGCTGCGTCAGATCGTA |
| | | | TTGAACTGCGGCGAAGAACCGTCTGTTG |
| | | | TTGCTAACACCGTTAAAGGCGGCGACGG |
| | | | CAACTACGGTTACAACGCAGCAACCGAA |
| | | | GAATACGGCAACATGATCGACATGGGTA |
| | | | TCCTGGATCCAACCAAAGTAACTCGTTC |
| | | | TGCTCTGCAGTACGCAGCTTCTGTGGCT |
| | | | GGCCTGATGATCACCACCGAATGCATGG |
| | | | TTACCGACCTGCCGAAAAACGATGCAGC |
| | | | TGACTTAGGCGCTGCTGGCGGTATGGGC |
| | | | GGCATGATGTAAGTTTAAACGCGGCCGC |
| | | | AATTTGAACGCCAGCACATGGACTCTCG |
| | | | AGTCTACTAGCGCAGCTTAATTAACCTA |
| | | | GGCTGCTGCCACCGCTGAGCAATAACTA |
| | | | GCATAACCCCTTGGGGCCTCTAAACGGG |
| | | | TCTTGAGGGGTTTTTTGCTGAAACCTCA |
| | | | GGCATTTGAGAAGCACACGGTCACACTG |
| | | | CTTCCGGTAGTCAATAAACCGGTAAACC |
| | | | AGCAATAGACATAAGCGGTGCATAATGT |
| | | | GCCTGTCAAATGGACGAAGCAGGGATTC |
| | | | TGCAAACCCTATGCTACTCCGTCAAGCC |
| | | | GTCAATTGTCTGATTCGTTACCAATTAT |
| | | | GACAACTTGACGGCTACATCATTCACTT |
| | | | TTTCTTCACAACCGGCACGGAACTCGCT |
| | | | CGGGCTGGCCCCGGTGCATTTTTTAAAT |
| | | | ACCCGCGAGAAATAGAGTTGATCGTCAA |
| | | | AACCAACATTGCGACCGACGGTGGCGAT |
| | | | AGGCATCCGGGTGGTGCTCAAAAGCAGC |
| | | | TTCGCCTGGCTGATACGTTGGTCCTCGC |
| | | | GCCAGCTTAAGACGCTAATCCCTAACTG |
| | | | CTGGCGGAAAAGATGTGACAGACGCGAC |
| | | | GGCGACAAGCAAACATGCTGTGCGACGC |
| | | | TGGCGATATCAAAATTGCTGTCTGCCAG |
| | | | GTGATCGCTGATGTACTGACAAGCCTCG |
| | | | CGTACCCGATTATCCATCGGTGGATGGA |
| | | | GCGACTCGTTAATCGCTTCCATGCGCCG |
| | | | CAGTAACAATTGCTCAAGCAGATTTATC |
| | | | GCCAGCAGCTCCGAATAGCGCCCTTCCC |
| | | | CTTGCCCGGCGTTAATGATTTGCCCAAA |
| | | | CAGGTCGCTGAAATGCGGCTGGTGCGCT |
| | | | TCATCCGGGCGAAAGAACCCCGTATTGG |
| | | | CAAATATTGACGGCCAGTTAAGCCATTC |
| | | | ATGCCAGTAGGCGCGCGGACGAAAGTAA |
| | | | ACCCACTGGTGATACCATTCGCGAGCCT |
| | | | CCGGATGACGACCGTAGTGATGAATCTC |
| | | | TCCTGGCGGGAACAGCAAAATATCACCC |
| | | | GGTCGGCAAACAAATTCTCGTCCCTGAT |
| | | | TTTTCACCACCCCCTGACCGCGAATGGT |
| | | | GAGATTGAGAATATAACCTTTCATTCCC |
| | | | AGCGGTCGGTCGATAAAAAAATCGAGAT |
| | | | AACCGTTGGCCTCAATCGGCGTTAAACC |
| | | | CGCCACCAGATGGGCATTAAACGAGTAT |
| | | | CCCGGCAGCAGGGGATCATTTTGCGCTT |
| | | | CAGCCATACTTTTCATACTCCCGCCATT |
| | | | CAGAGAAGAAACCAATTGTCCATATTGC |
| | | | ATCAGACATTGCCGTCACTGCGTCTTTT |
| | | | ACTGGCTCTTCTCGCTAACCAAACCGGT |
| | | | AACCCCGCTTATTAAAAGCATTCTGTAA |
| | | | CAAAGCGGGACCAAAGCCATGACAAAAA |
| | | | CGCGTAACAAAAGTGTCTATAATCACGG |
| | | | CAGAAAAGTCCACATTGATTATTTGCAC |
| | | | GGCGTCACACTTTGCTATGCCATAGCAT |
| | | | TTTTATCCATAAGATTAGCGGATCCTAC |
| | | | CTGACGCTTTTTATCGCAACTCTGGACA |
| | | | ATGTCTCCATACCCGTTTTTTTGGGCGA |
| | | | CCTCGTCGGAGGTTGTATGTCCGGTGTT |
| | | | CCGTGACGTCATCGGGCATTCATCATTC |
| | | | ATAGAATGTGTTACGGAGGAAACAAGTA |
| | | | ATGGCACTTAGCACCGCAACCAAGGCCG |
| | | | CGACGGACGCGCTGGCTGCCAATCGGGC |
| | | | ACCCACCAGCGTGAATGCACAGGAAGTG |
| | | | CACCGTTGGCTCCAGAGCTTCAACTGGG |
| | | | ATTTCAAGAACAACCGGACCAAGTACGC |
| | | | CACCAAGTACAAGATGGCGAACGAGACC |
| | | | AAGGAACAGTTCAAGCTGATCGCCAAGG |
| | | | AATATGCGCGCATGGAGGCAGTCAAGGA |
| | | | CGAAAGGCAGTTCGGTAGCCTGCAGGAT |
| | | | GCGCTGACCCGCCTCAACGCCGGTGTTC |
| | | | GCGTTCATCCGAAGTGGAACGAGACCAT |
| | | | GAAAGTGGTTTCGAACTTCCTGGAAGTG |
| | | | GGCGAATACAACGCCATCGCCGCTACCG |
| | | | GGATGCTGTGGGATTCCGCCCAGGCGGC |
| | | | GGAACAGAAGAACGGCTATCTGGCCCAG |
| | | | GTGTTGGATGAAATCCGCCACACCCACC |
| | | | AGTGTGCCTACGTCAACTACTACTTCGC |
| | | | GAAGAACGGCCAGGACCCGGCCGGTCAC |
| | | | AACGATGCTCGCCGCACCCGTACCATCG |
| | | | GTCCGCTGTGGAAGGGCATGAAGCGCGT |
| | | | GTTTTCCGACGGCTTCATTTCCGGCGAC |
| | | | GCCGTGGAATGCTCCCTCAACCTGCAGC |
| | | | TGGTGGGTGAGGCCTGCTTCACCAATCC |
| | | | GCTGATCGTCGCAGTGACCGAATGGGCT |
| | | | GCCGCCAACGGCGATGAAATCACCCCGA |
| | | | CGGTGTTCCTGTCGATCGAGACCGACGA |
| | | | ACTGCGCCACATGGCCAACGGTTACCAG |
| | | | ACCGTCGTTTCCATCGCCAACGATCCGG |
| | | | CTTCCGCCAAGTATCTCAACACGGACCT |
| | | | GAACAACGCCTTCTGGACCCAGCAGAAG |
| | | | TACTTCACGCCGGTGTTGGGCATGCTGT |
| | | | TCGAGTATGGCTCCAAGTTCAAGGTCGA |
| | | | GCCGTGGGTCAAGACGTGGAACCGCTGG |
| | | | GTGTACGAGGACTGGGGCGGCATCTGGA |
| | | | TCGGCCGTCTGGGCAAGTACGGGGTGGA |
| | | | GTCGCCGCGCAGCCTCAAGGACGCCAAG |
| | | | CAGGACGCTTACTGGGCTCACCACGACC |
| | | | TGTATCTGCTGGCTTATGCGCTGTGGCC |
| | | | GACCGGCTTCTTCCGTCTGGCGCTGCCG |
| | | | GATCAGGAAGAAATGGAGTGGTTCGAGG |
| | | | CCAACTACCCCGGCTGGTACGACCACTA |
| | | | CGGCAAGATCTACGAGGAATGGCGCGCC |
| | | | CGCGGTTGCGAGGATCCGTCCTCGGGCT |
| | | | TCATCCCGCTGATGTGGTTCATCGAAAA |
| | | | CAACCATCCCATCTACATCGATCGCGTG |
| | | | TCGCAAGTGCCGTTCTGCCCGAGCTTGG |
| | | | CCAAGGGCGCCAGCACCCTGCGCGTGCA |
| | | | CGAGTACAACGGCCAGATGCACACCTTC |
| | | | AGCGACCAGTGGGGCGAGCGCATGTGGC |
| | | | TGGCCGAGCCGGAGCGCTACGAGTGCCA |
| | | | GAACATCTTCGAACAGTACGAAGGACGC |
| | | | GAACTGTCGGAAGTGATCGCCGAACTGC |
| | | | ACGGGCTGCGCAGTGATGGCAAGACCCT |
| | | | GATCGCCCAGCCGCATGTCCGTGGCGAC |
| | | | AAGCTGTGGACGTTGGACGATATCAAAC |
| | | | GCCTGAACTGCGTCTTCAAGAACCCGGT |
| | | | GAAGGCATTCAATTGAAACGGGTGTCGG |
| | | | GCTCCGTCACAGGGCGGGGCCCGACGCA |
| | | | CGATCGTTCGATCAACCTCAAACCAAAA |
| | | | AGGAACATCGATATGAGCATGTTAGGAG |
| | | | AAAGACGCCGCGGTCTGACCGATCCGGA |
| | | | AATGGCGGCCGTCATTTTGAAGGCGCTT |
| | | | CCTGAAGCTCCGCTGGACGGCAACAACA |
| | | | AGATGGGTTATTTCGTCACCCCCCGCTG |
| | | | GAAACGCTTGACGGAATATGAAGCCCTG |
| | | | ACCGTTTATGCGCAGCCCAACGCCGACT |
| | | | GGATCGCCGGCGGCCTGGACTGGGGCGA |
| | | | CTGGACCCAGAAATTCCACGGCGGCCGC |
| | | | CCTTCCTGGGGCAACGAGACCACGGAGC |
| | | | TGCGCACCGTCGACTGGTTCAAGCACCG |
| | | | TGACCCGCTCCGCCGTTGGCATGCGCCG |
| | | | TACGTCAAGGACAAGGCCGAGGAATGGC |
| | | | GCTACACCGACCGCTTCCTGCAGGGTTA |
| | | | CTCCGCCGACGGTCAGATCCGGGCGATG |
| | | | AACCCGACCTGGCGGGACGAGTTCATCA |
| | | | ACCGGTATTGGGGCGCCTTCCTGTTCAA |
| | | | CGAATACGGATTGTTCAACGCTCATTCG |
| | | | CAGGGCGCCCGGGAGGCGCTGTCGGACG |
| | | | TAACCCGCGTCAGCCTGGCTTTCTGGGG |
| | | | CTTCGACAAGATCGACATCGCCCAGATG |
| | | | ATCCAACTCGAACGGGGTTTCCTCGCCA |
| | | | AGATCGTACCCGGTTTCGACGAGTCCAC |
| | | | AGCGGTGCCGAAGGCCGAATGGACGAAC |
| | | | GGGGAGGTCTACAAGAGCGCCCGTCTGG |
| | | | CCGTGGAAGGGCTGTGGCAGGAGGTGTT |
| | | | CGACTGGAACGAGAGCGCTTTCTCGGTG |
| | | | CACGCCGTCTATGACGCGCTGTTCGGTC |
| | | | AGTTCGTCCGCCGCGAGTTCTTTCAGCG |
| | | | GCTGGCTCCCCGCTTCGGCGACAATCTG |
| | | | ACGCCATTCTTCATCAACCAGGCCCAGA |
| | | | CATACTTCCAGATCGCCAAGCAGGGCGT |
| | | | ACAGGATCTGTATTACAACTGTCTGGGT |
| | | | GACGATCCGGAGTTCAGCGATTACAACC |
| | | | GTACCGTGATGCGCAACTGGACCGGCAA |
| | | | GTGGCTGGAGCCCACGATCGCCGCTCTG |
| | | | CGCGACTTCATGGGGCTGTTTGCGAAGC |
| | | | TGCCGGCGGGCACCACTGACAAGGAAGA |
| | | | AATCACCGCGTCCCTGTACCGGGTGGTC |
| | | | GACGACTGGATCGAGGACTACGCCAGCA |
| | | | GGATCGACTTCAAGGCGGACCGCGATCA |
| | | | GATCGTTAAAGCGGTTCTGGCAGGATTG |
| | | | AAATAATAGAGGAACTATTACGATGAGC |
| | | | GTAAACAGCAACGCATACGACGCCGGCA |
| | | | TCATGGGCCTGAAAGGCAAGGACTTCGC |
| | | | CGATCAGTTCTTTGCCGACGAAAACCAA |
| | | | GTGGTCCATGAAAGCGACACGGTCGTTC |
| | | | TGGTCCTCAAGAAGTCGGACGAGATCAA |
| | | | TACCTTTATCGAGGAGATCCTTCTGACG |
| | | | GACTACAAGAAGAACGTCAATCCGACGG |
| | | | TAAACGTGGAAGACCGCGCGGGTTACTG |
| | | | GTGGATCAAGGCCAACGGCAAGATCGAG |
| | | | GTCGATTGCGACGAGATTTCCGAGCTGT |
| | | | TGGGGCGGCAGTTCAACGTCTACGACTT |
| | | | CCTCGTCGACGTTTCCTCCACCATCGGC |
| | | | CGGGCCTATACCCTGGGCAACAAGTTCA |
| | | | CCATTACCAGTGAGCTGATGGGCCTGGA |
| | | | CCGCAAGCTCGAAGACTATCACGCTTAA |
| | | | GGAGAATGACATGGCGAAACTGGGTATA |
| | | | CACAGCAACGACACCCGCGACGCCTGGG |
| | | | TGAACAAGATCGCGCAGCTCAACACCCT |
| | | | GGAAAAAGCGGCCGAGATGCTGAAGCAG |
| | | | TTCCGGATGGACCACACCACGCCGTTCC |
| | | | GCAACAGCTACGAACTGGACAACGACTA |
| | | | CCTCTGGATCGAGGCCAAGCTCGAAGAG |
| | | | AAGGTCGCCGTCCTCAAGGCACGCGCCT |
| | | | TCAACGAGGTGGACTTCCGTCATAAGAC |
| | | | CGCTTTCGGCGAGGATGCCAAGTCCGTT |
| | | | CTGGACGGCACCGTCGCGAAGATGAACG |
| | | | CGGCCAAGGACAAGTGGGAGGCGGAGAA |
| | | | GATCCATATCGGTTTCCGCCAGGCCTAC |
| | | | AAGCCGCCGATCATGCCGGTGAACTATT |
| | | | TCCTGGACGGCGAGCGTCAGTTGGGGAC |
| | | | CCGGCTGATGGAACTGCGCAACCTCAAC |
| | | | TACTACGACACGCCGCTGGAAGAACTGC |
| | | | GCAAACAGCGCGGTGTGCGGGTGGTGCA |
| | | | TCTGCAGTCGCCGCACTGAAGGGAGGAA |
| | | | GTCTCGCCCTGGACGCGACGGCATCGCC |
| | | | GTGAAGTCCAGGGGGCAGGGATGCCGTT |
| | | | CCGGGCCGGCAGGCTGGCCCGGAATCTC |
| | | | TGGTTTTCAGGGGGCGTGCCGGTCCACG |
| | | | GCTCCCCCCTCCATCTTTCGTAAGGAAA |
| | | | TCACCATGGTCGAATCGGCATTTCAGCC |
| | | | ATTTTCGGGCGACGCAGACGAATGGTTC |
| | | | GAGGAACCACGGCCCCAGGCCGGTTTCT |
| | | | TCCCTTCCGCGGACTGGCATCTGCTCAA |
| | | | ACGGGACGAGACCTACGCAGCCTATGCC |
| | | | AAGGATCTCGATTTCATGTGGCGGTGGG |
| | | | TCATCGTCCGGGAAGAAAGGATCGTCCA |
| | | | GGAGGGTTGCTCGATCAGCCTGGAGTCG |
| | | | TCGATCCGCGCCGTGACGCACGTACTGA |
| | | | ATTATTTTGGTATGACCGAACAACGCGC |
| | | | CCCGGCAGAGGACCGGACCGGCGGAGTT |
| | | | CAACATTGAACAGGTAAGTTTATGCAGC |
| | | | GAGTTCACACTATCACGGCGGTGACGGA |
| | | | GGATGGCGAATCGCTCCGCTTCGAATGC |
| | | | CGTTCGGACGAGGACGTCATCACCGCCG |
| | | | CCCTGCGCCAGAACATCTTTCTGATGTC |
| | | | GTCCTGCCGGGAGGGCGGCTGTGCGACC |
| | | | TGCAAGGCCTTGTGCAGCGAAGGGGACT |
| | | | ACGACCTCAAGGGCTGCAGCGTTCAGGC |
| | | | GCTGCCGCCGGAAGAGGAGGAGGAAGGG |
| | | | TTGGTGTTGTTGTGCCGGACCTACCCGA |
| | | | AGACCGACCTGGAAATCGAACTGCCCTA |
| | | | TACCCATTGCCGCATCAGTTTTGGTGAG |
| | | | GTCGGCAGTTTCGAGGCGGAGGTCGTCG |
| | | | GCCTCAACTGGGTTTCGAGCAACACCGT |
| | | | CCAGTTTCTTTTGCAGAAGCGGCCCGAC |
| | | | GAGTGCGGCAACCGTGGCGTGAAATTCG |
| | | | AACCCGGTCAGTTCATGGACCTGACCAT |
| | | | CCCCGGCACCGATGTCTCCCGCTCCTAC |
| | | | TCGCCGGCGAACCTTCCTAATCCCGAAG |
| | | | GCCGCCTGGAGTTCCTGATCCGCGTGTT |
| | | | ACCGGAGGGACGGTTTTCGGACTACCTG |
| | | | CGCAATGACGCGCGTGTCGGACAGGTCC |
| | | | TCTCGGTCAAAGGGCCACTGGGCGTGTT |
| | | | CGGTCTCAAGGAGCGGGGCATGGCGCCG |
| | | | CGCTATTTCGTGGCCGGCGGCACCGGGT |
| | | | TGGCGCCGGTGGTCTCGATGGTGCGGCA |
| | | | GATGCAGGAGTGGACCGCGCCGAACGAG |
| | | | ACCCGCATCTATTTCGGTGTGAACACCG |
| | | | AGCCGGAATTGTTCTACATCGACGAGCT |
| | | | CAAATCCCTGGAACGATCGATGCGCAAT |
| | | | CTCACCGTGAAGGCCTGTGTCTGGCACC |
| | | | CGAGCGGGGACTGGGAAGGCGAGCAGGG |
| | | | CTCGCCCATCGATGCGTTGCGGGAAGAC |
| | | | CTGGAGTCCTCCGACGCCAACCCGGACA |
| | | | TTTATTTGTGCGGTCCGCCGGGCATGAT |
| | | | CGATGCCGCCTGCGAGCTGGTACGCAGC |
| | | | CGCGGTATCCCCGGCGAACAGGTCTTCT |
| | | | TCGAAAAATTCCTGCCGTCCGGGGCGGC |
| | | | CTGAACCGGGGAAGTACCGTGACCACCG |
| | | | AGCAGTTCCCGCCCCAATTCCTGCGTGA |
| | | | AATGATCGAGCAGCTGGACGCCAGCATC |
| | | | CAGGAGCTCGCACGCAAGGAAAAGGGAC |
| | | | TTGCGGCATCCCTGGGCACGGGCCGGGT |
| | | | CGCCGAGCTCAAGGAATACTGGGACCAC |
| | | | GTTGTTACAACCAATTAACCAATTCTGA |
| | | | CTATTTAACGACCCTGCCCTGAACCGAC |
| | | | GACCGGGTCATCGTGGCCGGATCTTGCG |
| | | | GCCCCTCGGCTTGAACGAATTGTTAGAC |
| | | | ATTATTTGCCGACTACCTTGGTGATCTC |
| | | | GCCTTTCACGTAGTGGACAAATTCTTCC |
| | | | AACTGATCTGCGCGCGAGGCCAAGCGAT |
| | | | CTTCTTCTTGTCCAAGATAAGCCTGTCT |
| | | | AGCTTCAAGTATGACGGGCTGATACTGG |
| | | | GCCGGCAGGCGCTCCATTGCCCAGTCGG |
| | | | CAGCGACATCCTTCGGCGCGATTTTGCC |
| | | | GGTTACTGCGCTGTACCAAATGCGGGAC |
| | | | AACGTAAGCACTACATTTCGCTCATCGC |
| | | | CAGCCCAGTCGGGCGGCGAGTTCCATAG |
| | | | CGTTAAGGTTTCATTTAGCGCCTCAAAT |
| | | | AGATCCTGTTCAGGAACCGGATCAAAGA |
| | | | GTTCCTCCGCCGCTGGACCTACCAAGGC |
| | | | AACGCTATGTTCTCTTGCTTTTGTCAGC |
| | | | AAGATAGCCAGATCAATGTCGATCGTGG |
| | | | CTGGCTCGAAGATACCTGCAAGAATGTC |
| | | | ATTGCGCTGCCATTCTCCAAATTGCAGT |
| | | | TCGCGCTTAGCTGGATAACGCCACGGAA |
| | | | TGATGTCGTCGTGCACAACAATGGTGAC |
| | | | TTCTACAGCGCGGAGAATCTCGCTCTCT |
| | | | CCAGGGGAAGCCGAAGTTTCCAAAAGGT |
| | | | CGTTGATCAAAGCTCGCCGCG |
| 37. | | pLC130 | GGCGGGTCGCTCCCTCTTGCGCTCTCCT |
| | | | GTTCCGACCCTGCCGTTTACCGGATACC |
| | | | TGTTCCGCCTTTCTCCCTTACGGGAAGT |
| | | | GTGGCGCTTTCTCATAGCTCACACACTG |
| | | | GTATCTCGGCTCGGTGTAGGTCGTTCGC |
| | | | TCCAAGCTGGGCTGTAAGCAAGAACTCC |
| | | | CCGTTCAGCCCGACTGCTGCGCCTTATC |
| | | | CGGTAACTGTTCACTTGAGTCCAACCCG |
| | | | GAAAAGCACGGTAAAACGCCACTGGCAG |
| | | | CAGCCATTGGTAACTGGGAGTTCGCAGA |
| | | | GGATTTGTTTAGCTAAACACGCGGTTGC |
| | | | TCTTGAAGTGTGCGCCAAAGTCCGGCTA |
| | | | CACTGGAAGGACAGATTTGGTTGCTGTG |
| | | | CTCTGCGAAAGCCAGTTACCACGGTTAA |
| | | | GCAGTTCCCCAACTGACTTAACCTTCGA |
| | | | TCAAACCACCTCCCCAGGTGGTTTTTTC |
| | | | GTTTACAGGGCAAAAGATTACGCGCAGA |
| | | | AAAAAAGGATCTCAAGAAGATCCTTTGA |
| | | | TCTTTTCTACTGAACCGCTCTAGATTTC |
| | | | AGTGCAATTTATCTCTTCAAATGTAGCA |
| | | | CCTGAAGTCAGCCCCATACGATATAAGT |
| | | | TGTAATTCTCATGTTAGTCATGCCCCGC |
| | | | GCCCACCGGAAGGAGCTGACTGGGTTGA |
| | | | AGGCTCTCAAGGGCATCGGTCGAGATCC |
| | | | CGGTGCCTAATGAGTGAGCTAACTTCGT |
| | | | CAGGATGGCCTTCTGCTTAATTTGATGC |
| | | | CTGGCAGTTTATGGCGGGCGTCCTGCCC |
| | | | GCCACCCTCCGGGCCGTTGCTTCGCAAC |
| | | | GTTCAAATCCGCTCCCGGCGGATTTGTC |
| | | | CTACTCAGGAGAGCGTTCACCGACAAAC |
| | | | AACAGATAAAACGAAAGGCCCAGTCTTT |
| | | | CGACTGAGCCTTTCGTTTTATTTGATGC |
| | | | CTGGCAGTTCCCTACTCTCGCATGGGGA |
| | | | GACCCCACACTACCATCGGCGCTACGGC |
| | | | GTTTCACTTCTGAGTTCGGCATGGGGTC |
| | | | AGGTGGGACCACCGCGCTACTGCCGCCA |
| | | | GGCAAATTCTGTTTTATCAGACCGCTTC |
| | | | TGCGTTCTGATTTAATCTGTATCAGGCT |
| | | | TTACATCGCATTTTTAATAATTTGGATG |
| | | | ACTTCTTCTAACTTAGGTTTACGAGGAT |
| | | | TTGTTAATGCACATGCATCTTTCATCGC |
| | | | ATTCTTAGCTAAAGTCTCAATGTCTTCT |
| | | | TCTTTAGCACCTAGTTCTTTAAAGCCTT |
| | | | TTGGAATGTTAAGGTCTTTAGCCATTCT |
| | | | TTCGATCGCTTTAATAGCTTTTTCAGCT |
| | | | GCATCGTACGTACTTAGACCGTCGACAT |
| | | | TTTCACCAAGAAAAGCAGCGATTTCTGC |
| | | | ATAACGTTCCACTTTAGAAATTAAGTTA |
| | | | AATCGACATACATATGGCAGAAGGACCG |
| | | | CATTGCAAACGCCATGAGGGAAGTTGTA |
| | | | GAATCCTCCTAATTGGTGTGCAATCGCA |
| | | | TGAACATAGCCTAAACCCGCGTTATTGA |
| | | | ATGCCATGCCAGCTAATGATTGAGCGAA |
| | | | GGCCATTTGTTCACGTGCTTCAATGTCT |
| | | | TTTCCATTTGCAACTGCACGCGGCAAGT |
| | | | ATTTAGAAATGATTTTGATCGCCTGAAT |
| | | | TGCAAGTGCATCTGTAATTGGAGTAGCA |
| | | | CCAGTTGAAACATATGCTTCAATTGCAT |
| | | | GAGTTAATGCATCTAATCCAGTAGCAGC |
| | | | AGTTAAGGACGGAGGCATTCCAACCATT |
| | | | AGCTCTGGGTCGTTGATTGAAAGTGTAG |
| | | | GTGTTACATGTTTATCCACAATGGCCAT |
| | | | TTTCACTTTGCGTTCAGTATCTGTGATG |
| | | | ATTGTGAATTTAGTTAATTCACTGCCTG |
| | | | TACCAGCTGTTGTATTAATCGCAATTAG |
| | | | CGGGACCATTGGTTCTTTTGATACATCG |
| | | | ACACCTTCATAATCGTGAATTTTTCCAC |
| | | | CATTAGCAGCTACTAATGCAATGGCTTT |
| | | | TCCGGCATCATGTGAACTTCCGCCGCCC |
| | | | AGAGTGACAATGCTGTCACAGTTTTCAG |
| | | | CGTTATACGCTTCTAAACCTTCTGCGAC |
| | | | GTTTTTATCGGTTGGATTTGGTTCGGCT |
| | | | TTTGGAAAAATGGATACTTCCACACCAG |
| | | | CTGCACGAATAATACTGGAAATTTTTTC |
| | | | AGAAAGACCTAAACCGTGAAGACCAGCA |
| | | | TCTGTAACTAATAAAGCTTTTTTCACAC |
| | | | CAAGATCAGCTAATCGAGTTCCAACCTC |
| | | | ATTAACTGATCCTGCACCAAATAGATTG |
| | | | ACTGAAGGCATAAAAAATGCACTTTGAG |
| | | | TGTTTGTCATTAATATCCTCCTTATTGT |
| | | | AACCTCTGAAGAAACCGGCAACTTACTC |
| | | | CAGATTCGCATGGCGACCATACATCGTT |
| | | | TTGGTATCCAGGCCTTTCTTTTCCATAA |
| | | | AACGCAGAATAACCGCGTCATAGAAGAG |
| | | | CAGCAGCGTTTGTTCAAATAAGCTACCC |
| | | | ATCGGCTGAATTGTCTCACGCGCTTCAG |
| | | | ATTTATCTTTCGGGCTACCCGGCATCTT |
| | | | GATGACGATGTCAGCGAGCTGCCCAATC |
| | | | GTGCTTTCGGGGTTGATGGTCACGGCTG |
| | | | CAATCGTTCCTCCGATACTCTTGGCTTT |
| | | | CTGGGCCATGCTCACCAGGCTTTTGGTT |
| | | | TCGCCAGAACCGCTACCAATAATCAAAA |
| | | | TGTCCTCTTTTTCGTAGTTGGGCGTCAC |
| | | | AGTTTCTCCAACCACGTATGCATCGATT |
| | | | CCCATGTGCATCATACGCATCGCGAAAC |
| | | | TCTTTGCCATGAAGCCAGAGCGGCCAGC |
| | | | GCCAGCAACGAAAACTTTTTTCGACTGC |
| | | | AGGATCCCGTTCACCAGCGCTTCTGCTT |
| | | | CTTCATCCGCAATCTGGTTTACACTGCT |
| | | | GTTCAGTTCCTTTACAATTTCCGCCAAA |
| | | | AACTCTGTAGTCAACATACTAATCATTA |
| | | | TTATCCTCCTATATCCTATAACGGTACA |
| | | | GCTTCAGGCTAGTTACAGCCCTTGCTTA |
| | | | ACCAGTTTGTTAATCTTTTCGGCCGCTG |
| | | | CCTTCTTGTCCGTTTGATTTGCGATCCC |
| | | | GCCGCCTACAATGACCAAATCCGGTTCA |
| | | | GCTTTGATAACCTCTGGCAGGGTTTCGA |
| | | | GCTTAATGCCGCCCGCGATGGCCGTTTT |
| | | | GGCATTTTTCACCACGGCCTTGATGCGT |
| | | | TTCAGGTCATCCAACGGGTTTTTCCCCA |
| | | | CCGCTTGAAGATCGTAACCCGCGTGCAC |
| | | | ACAAATATAATCCACGCCCATTTCGTCG |
| | | | ACCTGTTTCGCGCGTTCCTCCAGGTTTT |
| | | | TCACCGCGATCATGTCTACTAAGATCTT |
| | | | CTTGCCCAGTTTTTTTGCTTCTTCAACC |
| | | | GCACCTTTAATGGAAACATCCTCCGCTG |
| | | | CAGCTAAAATGGTCACAATATCCGCACC |
| | | | GTGTTCCGCCGCTTTAGCAACTTCGTAC |
| | | | GCCGCCGCATCCATCGTCTTCATATCGG |
| | | | CCAGAACCTGCAGATGCGGAAAGGCGTC |
| | | | CTTCACCGCTTTCACGGCCTGCAGGCCC |
| | | | CAGATCTTAATCACCGGTGTACCAATCT |
| | | | CGACAATATCCACATACTCCTGCACTTC |
| | | | GGCCACGACCTGTTTTGCTTCTTCGATG |
| | | | TTAACTAAGTCTAACGCTAACTGAAGTT |
| | | | CCATTATATTCCTCCTTTATGGCCCTCG |
| | | | CGAGTACAGTTATGCCCAAAAAAACGGG |
| | | | TATGGAGAAACAGTAGAGAGTTGCGATA |
| | | | AAAAGCGTCAGGTAGGATCCGCTAATCT |
| | | | TATGGATAAAAATGCTATGGCATAGCAA |
| | | | AGTGTGACGCCGTGCAAATAATCAATGT |
| | | | GGACTTTTCTGCCGTGATTATAGACACT |
| | | | TTTGTTACGCGTTTTTGTCATGGCTTTG |
| | | | GTCCCGCTTTGTTACAGAATGCTTTTAA |
| | | | TAAGCGGGGTTACCGGTTTGGTTAGCGA |
| | | | GAAGAGCCAGTAAAAGACGCAGTGACGG |
| | | | CAATGTCTGATGCAATATGGACAATTGG |
| | | | TTTCTTCTCTGAATGGCGGGAGTATGAA |
| | | | AAGTATGGCTGAAGCGCAAAATGATCCC |
| | | | CTGCTGCCGGGATACTCGTTTAATGCCC |
| | | | ATCTGGTGGCGGGTTTAACGCCGATTGA |
| | | | GGCCAACGGTTATCTCGATTTTTTTATC |
| | | | GACCGACCGCTGGGAATGAAAGGTTATA |
| | | | TTCTCAATCTCACCATTCGCGGTCAGGG |
| | | | GGTGGTGAAAAATCAGGGACGAGAATTT |
| | | | GTTTGCCGACCGGGTGATATTTTGCTGT |
| | | | TCCCGCCAGGAGAGATTCATCACTACGG |
| | | | TCGTCATCCGGAGGCTCGCGAATGGTAT |
| | | | CACCAGTGGGTTTACTTTCGTCCGCGCG |
| | | | CCTACTGGCATGAATGGCTTAACTGGCC |
| | | | GTCAATATTTGCCAATACGGGGTTCTTT |
| | | | CGCCCGGATGAAGCGCACCAGCCGCATT |
| | | | TCAGCGACCTGTTTGGGCAAATCATTAA |
| | | | CGCCGGGCAAGGGGAAGGGCGCTATTCG |
| | | | GAGCTGCTGGCGATAAATCTGCTTGAGC |
| | | | AATTGTTACTGCGGCGCATGGAAGCGAT |
| | | | TAACGAGTCGCTCCATCCACCGATGGAT |
| | | | AATCGGGTACGCGAGGCTTGTCAGTACA |
| | | | TCAGCGATCACCTGGCAGACAGCAATTT |
| | | | TGATATCGCCAGCGTCGCACAGCATGTT |
| | | | TGCTTGTCGCCGTCGCGTCTGTCACATC |
| | | | TTTTCCGCCAGCAGTTAGGGATTAGCGT |
| | | | CTTAAGCTGGCGCGAGGACCAACGTATC |
| | | | AGCCAGGCGAAGCTGCTTTTGAGCACCA |
| | | | CCCGGATGCCTATCGCCACCGTCGGTCG |
| | | | CAATGTTGGTTTTGACGATCAACTCTAT |
| | | | TTCTCGCGGGTATTTAAAAAATGCACCG |
| | | | GGGCCAGCCCGAGCGAGTTCCGTGCCGG |
| | | | TTGTGAAGAAAAAGTGAATGATGTAGCC |
| | | | GTCAAGTTGTCATAATTGGTAACGAATC |
| | | | AGACAATTGACGGCTTGACGGAGTAGCA |
| | | | TAGGGTTTGCAGAATCCCTGCTTCGTCC |
| | | | ATTTGACAGGCACATTATGCATGCCGCT |
| | | | TCGCCTTCGCGCGCGAATTGATCTGCTG |
| | | | CCTCGCGCGTTTCGGTGATGACGGTGAA |
| | | | AACCTCTGACACATGCAGCTCCCGGAGA |
| | | | CGGTCACAGCTTGTCTGTAAGCGGATGC |
| | | | CGGGAGCAGACAAGCCCGTCAGGGCGCG |
| | | | TCAGCGGGTGTTGGCGGGTGTCGGGGCG |
| | | | CAGCCATGACCCAGTCACGTAGCGATAG |
| | | | CGGAGTGTATACTGGCTTAACTATGCGG |
| | | | CATCAGAGCAGATTGTACTGAGAGTGCA |
| | | | CCATATGCGGTGTGAAATACCGCACAGA |
| | | | TGCGTAAGGAGAGTCTACTAGCGCAGCT |
| | | | TAATTAACCTAGGCTGCTGCCACCGCTG |
| | | | AGCAATAACTAGCATAACCCCTTGGGGC |
| | | | CTCTAAACGGGTCTTGAGGGGTTTTTTG |
| | | | CTGAAACCTCAGGCATTTGAGAAGCACA |
| | | | CGGTCACACTGCTTCCGGTAGTCAATAA |
| | | | ACCGGTAAACCAGCAATAGACATAAGCG |
| | | | GCTATTTAACGACCCTGCCCTGAACCGA |
| | | | CGACCGGGTCATCGTGGCCGGATCTTGC |
| | | | GGCCCCTCGGCTTGAACGAATTGTTAGA |
| | | | CATTATTTGCCGACTACCTTGGTGATCT |
| | | | CGCCTTTCACGTAGTGGACAAATTCTTC |
| | | | CAACTGATCTGCGCGCGAGGCCAAGCGA |
| | | | TCTTCTTCTTGTCCAAGATAAGCCTGTC |
| | | | TAGCTTCAAGTATGACGGGCTGATACTG |
| | | | GGCCGGCAGGCGCTCCATTGCCCAGTCG |
| | | | GCAGCGACATCCTTCGGCGCGATTTTGC |
| | | | CGGTTACTGCGCTGTACCAAATGCGGGA |
| | | | CAACGTAAGCACTACATTTCGCTCATCG |
| | | | CCAGCCCAGTCGGGCGGCGAGTTCCATA |
| | | | GCGTTAAGGTTTCATTTAGCGCCTCAAA |
| | | | TAGATCCTGTTCAGGAACCGGATCAAAG |
| | | | AGTTCCTCCGCCGCTGGACCTACCAAGG |
| | | | CAACGCTATGTTCTCTTGCTTTTGTCAG |
| | | | CAAGATAGCCAGATCAATGTCGATCGTG |
| | | | GCTGGCTCGAAGATACCTGCAAGAATGT |
| | | | CATTGCGCTGCCATTCTCCAAATTGCAG |
| | | | TTCGCGCTTAGCTGGATAACGCCACGGA |
| | | | ATGATGTCGTCGTGCACAACAATGGTGA |
| | | | CTTCTACAGCGCGGAGAATCTCGCTCTC |
| | | | TCCAGGGGAAGCCGAAGTTTCCAAAAGG |
| | | | TCGTTGATCAAAGCTCGCCGCGTTGTTT |
| | | | CATCAAGCCTTACGGTCACCGTAACCAG |
| | | | CAAATCAATATCACTGTGTGGCTTCAGG |
| | | | CCGCCATCCACTGCGGAGCCGTACAAAT |
| | | | GTACGGCCAGCAACGTCGGTTCGAGATG |
| | | | GCGCTCGATGACGCCAACTACCTCTGAT |
| | | | AGTTGAGTCGATACTTCGGCGATCACCG |
| | | | CTTCCCTCATACTCTTCCTTTTTCAATA |
| | | | TTATTGAAGCATTTATCAGGGTTATTGT |
| | | | CTCATGAGCGGATACATATTTGAATGTA |
| | | | TTTAGAAAAATAAACAAATAGCTAGCTC |
| | | | ACTCGGTCGCTACGCTCCGGGCGTGAGA |
| | | | CTGCGGCGGGCGCTGCGGACACATACAA |
| | | | AGTTACCCACAGATTCCGTGGATAAGCA |
| | | | GGGGACTAACATGTGAGGCAAAACAGCA |
| | | | GGGCCGCGCCGGTGGCGTTTTTCCATAG |
| | | | GCTCCGCCCTCCTGCCAGAGTTCACATA |
| | | | AACAGACGCTTTTCCGGTGCATCTGTGG |
| | | | GAGCCGTGAGGCTCAACCATGAATCTGA |
| | | | CAGTACGGGCGAAACCCGACAGGACTTA |
| | | | AAGATCCCCACCGTTTCC |
| 38. | | pLC158 | TCTCCTTACGCATCTGTGCGGTATTTCA |
| | | | CACCGCATATGGTGCACTCTCAGTACAA |
| | | | TCTGCTCTGATGCCGCATAGTTAAGCCA |
| | | | GTATACACTCCGCTATCGCTACGTGACT |
| | | | GGGTCATGGCTGCGCCCCGACACCCGCC |
| | | | AACACCCGCTGACGCGCCCTGACGGGCT |
| | | | TGTCTGCTCCCGGCATCCGCTTACAGAC |
| | | | AAGCTGTGACCGTCTCCGGGAGCTGCAT |
| | | | GTGTCAGAGGTTTTCACCGTCATCACCG |
| | | | AAACGCGCGAGGCAGCAGATCAATTCGC |
| | | | GCGCGAAGGCGAAGCGGCATGCATAATG |
| | | | TGCCTGTCAAATGGACGAAGCAGGGATT |
| | | | CTGCAAACCCTATGCTACTCCGTCAAGC |
| | | | CGTCAATTGTCTGATTCGTTACCAATTA |
| | | | TGACAACTTGACGGCTACATCATTCACT |
| | | | TTTTCTTCACAACCGGCACGGAACTCGC |
| | | | TCGGGCTGGCCCCGGTGCATTTTTTAAA |
| | | | TACCCGCGAGAAATAGAGTTGATCGTCA |
| | | | AAACCAACATTGCGACCGACGGTGGCGA |
| | | | TAGGCATCCGGGTGGTGCTCAAAAGCAG |
| | | | CTTCGCCTGGCTGATACGTTGGTCCTCG |
| | | | CGCCAGCTTAAGACGCTAATCCCTAACT |
| | | | GCTGGCGGAAAAGATGTGACAGACGCGA |
| | | | CGGCGACAAGCAAACATGCTGTGCGACG |
| | | | CTGGCGATATCAAAATTGCTGTCTGCCA |
| | | | GGTGATCGCTGATGTACTGACAAGCCTC |
| | | | GCGTACCCGATTATCCATCGGTGGATGG |
| | | | AGCGACTCGTTAATCGCTTCCATGCGCC |
| | | | GCAGTAACAATTGCTCAAGCAGATTTAT |
| | | | CGCCAGCAGCTCCGAATAGCGCCCTTCC |
| | | | CCTTGCCCGGCGTTAATGATTTGCCCAA |
| | | | ACAGGTCGCTGAAATGCGGCTGGTGCGC |
| | | | TTCATCCGGGCGAAAGAACCCCGTATTG |
| | | | GCAAATATTGACGGCCAGTTAAGCCATT |
| | | | CATGCCAGTAGGCGCGCGGACGAAAGTA |
| | | | AACCCACTGGTGATACCATTCGCGAGCC |
| | | | TCCGGATGACGACCGTAGTGATGAATCT |
| | | | CTCCTGGCGGGAACAGCAAAATATCACC |
| | | | CGGTCGGCAAACAAATTCTCGTCCCTGA |
| | | | TTTTTCACCACCCCCTGACCGCGAATGG |
| | | | TGAGATTGAGAATATAACCTTTCATTCC |
| | | | CAGCGGTCGGTCGATAAAAAAATCGAGA |
| | | | TAACCGTTGGCCTCAATCGGCGTTAAAC |
| | | | CCGCCACCAGATGGGCATTAAACGAGTA |
| | | | TCCCGGCAGCAGGGGATCATTTTGCGCT |
| | | | TCAGCCATACTTTTCATACTCCCGCCAT |
| | | | TCAGAGAAGAAACCAATTGTCCATATTG |
| | | | CATCAGACATTGCCGTCACTGCGTCTTT |
| | | | TACTGGCTCTTCTCGCTAACCAAACCGG |
| | | | TAACCCCGCTTATTAAAAGCATTCTGTA |
| | | | ACAAAGCGGGACCAAAGCCATGACAAAA |
| | | | ACGCGTAACAAAAGTGTCTATAATCACG |
| | | | GCAGAAAAGTCCACATTGATTATTTGCA |
| | | | CGGCGTCACACTTTGCTATGCCATAGCA |
| | | | TTTTTATCCATAAGATTAGCGGATCCTA |
| | | | CCTGACGCTTTTTATCGCAACTCTCTAC |
| | | | TGTTTCTCCATACCCGTTTTTTTGGGCA |
| | | | TAACTGTACTCGCGAGGGCCATAAAGGA |
| | | | GGAATATAATGGAACTTCAGTTAGCGTT |
| | | | AGACTTAGTTAACATCGAAGAAGCAAAA |
| | | | CAGGTCGTGGCCGAAGTGCAGGAGTATG |
| | | | TGGATATTGTCGAGATTGGTACACCGGT |
| | | | GATTAAGATCTGGGGCCTGCAGGCCGTG |
| | | | AAAGCGGTGAAGGACGCCTTTCCGCATC |
| | | | TGCAGGTTCTGGCCGATATGAAGACGAT |
| | | | GGATGCGGCGGCGTACGAAGTTGCTAAA |
| | | | GCGGCGGAACACGGTGCGGATATTGTGA |
| | | | CCATTTTAGCTGCAGCGGAGGATGTTTC |
| | | | CATTAAAGGTGCGGTTGAAGAAGCAAAA |
| | | | AAACTGGGCAAGAAGATCTTAGTAGACA |
| | | | TGATCGCGGTGAAAAACCTGGAGGAACG |
| | | | CGCGAAACAGGTCGACGAAATGGGCGTG |
| | | | GATTATATTTGTGTGCACGCGGGTTACG |
| | | | ATCTTCAAGCGGTGGGGAAAAACCCGTT |
| | | | GGATGACCTGAAACGCATCAAGGCCGTG |
| | | | GTGAAAAATGCCAAAACGGCCATCGCGG |
| | | | GCGGCATTAAGCTCGAAACCCTGCCAGA |
| | | | GGTTATCAAAGCTGAACCGGATTTGGTC |
| | | | ATTGTAGGCGGCGGGATCGCAAATCAAA |
| | | | CGGACAAGAAGGCAGCGGCCGAAAAGAT |
| | | | TAACAAACTGGTTAAGCAAGGGCTGTAA |
| | | | CTAGCCTGAAGCTGTACCGTTATAGGAT |
| | | | ATAGGAGGATAATAATGATTAGTATGTT |
| | | | GACTACAGAGTTTTTGGCGGAAATTGTA |
| | | | AAGGAACTGAACAGCAGTGTAAACCAGA |
| | | | TTGCGGATGAAGAAGCAGAAGCGCTGGT |
| | | | GAACGGGATCCTGCAGTCGAAAAAAGTT |
| | | | TTCGTTGCTGGCGCTGGCCGCTCTGGCT |
| | | | TCATGGCAAAGAGTTTCGCGATGCGTAT |
| | | | GATGCACATGGGAATCGATGCATACGTG |
| | | | GTTGGAGAAACTGTGACGCCCAACTACG |
| | | | AAAAAGAGGACATTTTGATTATTGGTAG |
| | | | CGGTTCTGGCGAAACCAAAAGCCTGGTG |
| | | | AGCATGGCCCAGAAAGCCAAGAGTATCG |
| | | | GAGGAACGATTGCAGCCGTGACCATCAA |
| | | | CCCCGAAAGCACGATTGGGCAGCTCGCT |
| | | | GACATCGTCATCAAGATGCCGGGTAGCC |
| | | | CGAAAGATAAATCTGAAGCGCGTGAGAC |
| | | | AATTCAGCCGATGGGTAGCTTATTTGAA |
| | | | CAAACGCTGCTGCTCTTCTATGACGCGG |
| | | | TTATTCTGCGTTTTATGGAAAAGAAAGG |
| | | | CCTGGATACCAAAACGATGTATGGTCGC |
| | | | CATGCGAATCTGGAGTAAGTTGCCGGTT |
| | | | TCTTCAGAGGTTACAATAAGGAGGATAT |
| | | | TAATGACCACTGCTGCACCCCAAGAATT |
| | | | TACTGCTGCTGTTGTTGAAAAATTCGGT |
| | | | CATGACGTGACCGTGAAGGATATTGACC |
| | | | TTCCAAAGCCAGGGCCACACCAGGCATT |
| | | | GGTGAAGGTACTCACCTCCGGCATCTGC |
| | | | CACACCGACCTCCACGCCTTGGAGGGCG |
| | | | ATTGGCCAGTAAAGCCGGAACCACCATT |
| | | | CGTACCAGGACACGAAGGTGTAGGTGAA |
| | | | GTTGTTGAGCTCGGACCAGGTGAACACG |
| | | | ATGTGAAGGTCGGCGATATTGTCGGCAA |
| | | | TGCGTGGCTCTGGTCAGCGTGTGGCACC |
| | | | TGCGAATACTGCATCACCGGCAGGGAAA |
| | | | CTCAGTGCAACGAAGCTGAGTATGGTGG |
| | | | CTACACCCAAAATGGATCCTTCGGCCAG |
| | | | TACATGCTGGTGGATACCCGTTACGCCG |
| | | | CTCGCATCCCAGACGGCGTGGACTACCT |
| | | | CGAAGCAGCACCAATTCTGTGTGCAGGC |
| | | | GTGACTGTCTACAAGGCACTCAAAGTCT |
| | | | CTGAAACCCGCCCGGGCCAATTCATGGT |
| | | | GATCTCCGGTGTCGGCGGACTTGGCCAC |
| | | | ATCGCAGTCCAATACGCAGCGGCGATGG |
| | | | GCATGCGTGTCATTGCGGTAGATATTGC |
| | | | CGATGACAAGCTGGAACTTGCCCGTAAG |
| | | | CACGGTGCGGAATTTACCGTGAATGCGC |
| | | | GTAATGAAGATTCAGGCGAAGCTGTACA |
| | | | GAAGTACACCAACGGTGGCGCACACGGC |
| | | | GTGCTTGTGACTGCAGTTCACGAGGCAG |
| | | | CATTCGGCCAGGCACTGGATATGGCTCG |
| | | | ACGTGCAGGAACAATTGTGTTCAACGGT |
| | | | CTGCCACCGGGAGAGTTCCCAGCATCCG |
| | | | TGTTCAACATCGTATTCAAGGGCCTGAC |
| | | | CATCCGTGGATCCCTCGTGGGAACCCGC |
| | | | CAAGACTTGGCCGAAGCGCTCGATTTCT |
| | | | TTGCACGCGGACTAATCAAGCCAACCGT |
| | | | GAGTGAGTGCTCCCTCGATGAGGTCAAT |
| | | | GGTGTGCTTGACCGCATGCGAAACGGCA |
| | | | AGATTGATGGTCGTGTGGCAATTCGCTA |
| | | | CTAAAGCCTGATACAGATTAAATCAGAA |
| | | | CGCAGAAGCGGTCTGATAAAACAGAATT |
| | | | TGCCTGGCGGCAGTAGCGCGGTGGTCCC |
| | | | ACCTGACCCCATGCCGAACTCAGAAGTG |
| | | | AAACGCCGTAGCGCCGATGGTAGTGTGG |
| | | | GGTCTCCCCATGCGAGAGTAGGGAACTG |
| | | | CCAGGCATCAAATAAAACGAAAGGCTCA |
| | | | GTCGAAAGACTGGGCCTTTCGTTTTATC |
| | | | TGTTGTTTGTCGGTGAACGCTCTCCTGA |
| | | | GTAGGACAAATCCGCCGGGAGCGGATTT |
| | | | GAACGTTGCGAAGCAACGGCCCGGAGGG |
| | | | TGGCGGGCAGGACGCCCGCCATAAACTG |
| | | | CCAGGCATCAAATTAAGCAGAAGGCCAT |
| | | | CCTGACGAAGTTAGCTCACTCATTAGGC |
| | | | ACCGGGATCTCGACCGATGCCCTTGAGA |
| | | | GCCTTCAACCCAGTCAGCTCCTTCCGGT |
| | | | GGGCGCGGGGCATGACTAACATGAGAAT |
| | | | TACAACTTATATCGTATGGGGCTGACTT |
| | | | CAGGTGCTACATTTGAAGAGATAAATTG |
| | | | CACTGAAATCTAGAGCGGTTCAGTAGAA |
| | | | AAGATCAAAGGATCTTCTTGAGATCCTT |
| | | | TTTTTCTGCGCGTAATCTTTTGCCCTGT |
| | | | AAACGAAAAAACCACCTGGGGAGGTGGT |
| | | | TTGATCGAAGGTTAAGTCAGTTGGGGAA |
| | | | CTGCTTAACCGTGGTAACTGGCTTTCGC |
| | | | AGAGCACAGCAACCAAATCTGTCCTTCC |
| | | | AGTGTAGCCGGACTTTGGCGCACACTTC |
| | | | AAGAGCAACCGCGTGTTTAGCTAAACAA |
| | | | ATCCTCTGCGAACTCCCAGTTACCAATG |
| | | | GCTGCTGCCAGTGGCGTTTTACCGTGCT |
| | | | TTTCCGGGTTGGACTCAAGTGAACAGTT |
| | | | ACCGGATAAGGCGCAGCAGTCGGGCTGA |
| | | | ACGGGGAGTTCTTGCTTACAGCCCAGCT |
| | | | TGGAGCGAACGACCTACACCGAGCCGAG |
| | | | ATACCAGTGTGTGAGCTATGAGAAAGCG |
| | | | CCACACTTCCCGTAAGGGAGAAAGGCGG |
| | | | AACAGGTATCCGGTAAACGGCAGGGTCG |
| | | | GAACAGGAGAGCGCAAGAGGGAGCGACC |
| | | | CGCCGGAAACGGTGGGGATCTTTAAGTC |
| | | | CTGTCGGGTTTCGCCCGTACTGTCAGAT |
| | | | TCATGGTTGAGCCTCACGGCTCCCACAG |
| | | | ATGCACCGGAAAAGCGTCTGTTTATGTG |
| | | | AACTCTGGCAGGAGGGCGGAGCCTATGG |
| | | | AAAAACGCCACCGGCGCGGCCCTGCTGT |
| | | | TTTGCCTCACATGTTAGTCCCCTGCTTA |
| | | | TCCACGGAATCTGTGGGTAACTTTGTAT |
| | | | GTGTCCGCAGCGCCCGCCGCAGTCTCAC |
| | | | GCCCGGAGCGTAGCGACCGAGTGAGCTA |
| | | | GCTATTTGTTTATTTTTCTAAATACATT |
| | | | CAAATATGTATCCGCTCATGAGACAATA |
| | | | ACCCTGATAAATGCTTCAATAATATTGA |
| | | | AAAAGGAAGAGTATGAGGGAAGCGGTGA |
| | | | TCGCCGAAGTATCGACTCAACTATCAGA |
| | | | GGTAGTTGGCGTCATCGAGCGCCATCTC |
| | | | GAACCGACGTTGCTGGCCGTACATTTGT |
| | | | ACGGCTCCGCAGTGGATGGCGGCCTGAA |
| | | | GCCACACAGTGATATTGATTTGCTGGTT |
| | | | ACGGTGACCGTAAGGCTTGATGAAACAA |
| | | | CGCGGCGAGCTTTGATCAACGACCTTTT |
| | | | GGAAACTTCGGCTTCCCCTGGAGAGAGC |
| | | | GAGATTCTCCGCGCTGTAGAAGTCACCA |
| | | | TTGTTGTGCACGACGACATCATTCCGTG |
| | | | GCGTTATCCAGCTAAGCGCGAACTGCAA |
| | | | TTTGGAGAATGGCAGCGCAATGACATTC |
| | | | TTGCAGGTATCTTCGAGCCAGCCACGAT |
| | | | CGACATTGATCTGGCTATCTTGCTGACA |
| | | | AAAGCAAGAGAACATAGCGTTGCCTTGG |
| | | | TAGGTCCAGCGGCGGAGGAACTCTTTGA |
| | | | TCCGGTTCCTGAACAGGATCTATTTGAG |
| | | | GCGCTAAATGAAACCTTAACGCTATGGA |
| | | | ACTCGCCGCCCGACTGGGCTGGCGATGA |
| | | | GCGAAATGTAGTGCTTACGTTGTCCCGC |
| | | | ATTTGGTACAGCGCAGTAACCGGCAAAA |
| | | | TCGCGCCGAAGGATGTCGCTGCCGACTG |
| | | | GGCAATGGAGCGCCTGCCGGCCCAGTAT |
| | | | CAGCCCGTCATACTTGAAGCTAGACAGG |
| | | | CTTATCTTGGACAAGAAGAAGATCGCTT |
| | | | GGCCTCGCGCGCAGATCAGTTGGAAGAA |
| | | | TTTGTCCACTACGTGAAAGGCGAGATCA |
| | | | CCAAGGTAGTCGGCAAATAATGTCTAAC |
| | | | AATTCGTTCAAGCCGAGGGGCCGCAAGA |
| | | | TCCGGCCACGATGACCCGGTCGTCGGTT |
| | | | CAGGGCAGGGTCGTTAAATAGCCGCTTA |
| | | | TGTCTATTGCTGGTTTACCGGTTTATTG |
| | | | ACTACCGGAAGCAGTGTGACCGTGTGCT |
| | | | TCTCAAATGCCTGAGGTTTCAGCAAAAA |
| | | | ACCCCTCAAGACCCGTTTAGAGGCCCCA |
| | | | AGGGGTTATGCTAGTTATTGCTCAGCGG |
| | | | TGGCAGCAGCCTAGGTTAATTAAGCTGC |
| | | | GCTAGTAGAC |
| 39. | | pBZ27 | TAATGTGTAAAACATGTACATGCAGATT |
| | | | GCTGGGGGTGCAGGGGGCGGAGCCACCC |
| | | | TGTCCATGCGGGGTGTGGGGCTTGCCCC |
| | | | GCCGGTACAGACAGTGAGCACCGGGGCA |
| | | | CCTAGTCGCGGATACCCCCCCTAGGTAT |
| | | | CGGACACGTAACCCTCCCATGTCGATGC |
| | | | AAATCTTTAACATTGAGTACGGGTAAGC |
| | | | TGGCACGCATAGCCAAGCTAGGCGGCCA |
| | | | CCAAACACCACTAAAAATTAATAGTCCC |
| | | | TAGACAAGACAAACCCCCGTGCGAGCTA |
| | | | CCAACTCATATGCACGGGGGCCACATAA |
| | | | CCCGAAGGGGTTTCAATTGACAACCATA |
| | | | GCACTAGCTAAGACAACGGGCACAACAC |
| | | | CCGCACAAACTCGCACTGCGCAACCCCG |
| | | | CACAACATCGGGTCTAGGTAACACTGAA |
| | | | ATAGAAGTGAACACCTCTAAGGAACCGC |
| | | | AGGTCAATGAGGGTTCTAAGGTCACTCG |
| | | | CGCTAGGGCGTGGCGTAGGCAAAACGTC |
| | | | ATGTACAAGATCACCAATAGTAAGGCTC |
| | | | TGGCGGGGTGCCATAGGTGGCGCAGGGA |
| | | | CGAAGCTGTTGCGGTGTCCTGGTCGTCT |
| | | | AACGGTGCTTCGCAGTTTGAGGGTCTGC |
| | | | AAAACTCTCACTCTCGCTGGGGGTCACC |
| | | | TCTGGCTGAATTGGAAGTCATGGGCGAA |
| | | | CGCCGCATTGAGCTGGCTATTGCTACTA |
| | | | AGAATCACTTGGCGGCGGGTGGCGCGCT |
| | | | CATGATGTTTGTGGGCACTGTTCGACAC |
| | | | AACCGCTCACAGTCATTTGCGCAGGTTG |
| | | | AAGCGGGTATTAAGACTGCGTACTCTTC |
| | | | GATGGTGAAAACATCTCAGTGGAAGAAA |
| | | | GAACGTGCACGGTACGGGGTGGAGCACA |
| | | | CCTATAGTGACTATGAGGTCACAGACTC |
| | | | TTGGGCGAACGGTTGGCACTTGCACCGC |
| | | | AACATGCTGTTGTTCTTGGATCGTCCAC |
| | | | TGTCTGACGATGAACTCAAGGCGTTTGA |
| | | | GGATTCCATGTTTTCCCGCTGGTCTGCT |
| | | | GGTGTGGTTAAGGCCGGTATGGACGCGC |
| | | | CACTGCGTGAGCACGGGGTCAAACTTGA |
| | | | TCAGGTGTCTACCTGGGGTGGAGACGCT |
| | | | GCGAAAATGGCAACCTACCTCGCTAAGG |
| | | | GCATGTCTCAGGAACTGACTGGCTCCGC |
| | | | TACTAAAACCGCGTCTAAGGGGTCGTAC |
| | | | ACGCCGTTTCAGATGTTGGATATGTTGG |
| | | | CCGATCAAAGCGACGCCGGCGAGGATAT |
| | | | GGACGCTGTTTTGGTGGCTCGGTGGCGT |
| | | | GAGTATGAGGTTGGTTCTAAAAACCTGC |
| | | | GTTCGTCCTGGTCACGTGGGGCTAAGCG |
| | | | TGCTTTGGGCATTGATTACATAGACGCT |
| | | | GATGTACGTCGTGAAATGGAAGAAGAAC |
| | | | TGTACAAGCTCGCCGGTCTGGAAGCACC |
| | | | GGAACGGGTCGAATCAACCCGCGTTGCT |
| | | | GTTGCTTTGGTGAAGCCCGATGATTGGA |
| | | | AACTGATTCAGTCTGATTTCGCGGTTAG |
| | | | GCAGTACGTTCTAGATTGCGTGGATAAG |
| | | | GCTAAGGACGTGGCCGCTGCGCAACGTG |
| | | | TCGCTAATGAGGTGCTGGCAAGTCTGGG |
| | | | TGTGGATTCCACCCCGTGCATGATCGTT |
| | | | ATGGATGATGTGGACTTGGACGCGGTTC |
| | | | TGCCTACTCATGGGGACGCTACTAAGCG |
| | | | TGATCTGAATGCGGCGGTGTTCGCGGGT |
| | | | AATGAGCAGACTATTCTTCGCACCCACT |
| | | | AAAAGCGGCATAAACCCCGTTCGATATT |
| | | | TTGTGCGATGAATTTATGGTCAATGTCG |
| | | | CGGGGGCAAACTATGATGGGTCTTGTTG |
| | | | TTGCAGCCGAACGACCTAGCGCAGCGAG |
| | | | TCAGTGAGCGAGGAAGCGGAAGAGCGCC |
| | | | TGATGCGGTATTTTCTCCTTACGCATCT |
| | | | GTGCGGTATTTCACACCGCATATGGTGC |
| | | | ACTCTCAGTACAATCTGCTCTGATGCCG |
| | | | CATAGTTAAGCCAGTATACACTCCGCTA |
| | | | TCGCTACGTGACTGGGTCATGGCTGCGC |
| | | | CCCGACACCCGCCAACACCCGCTGACGC |
| | | | GCCCTGACGGGCTTGTCTGCTCCCGGCA |
| | | | TCCGCTTACAGACAAGCTGTGACCGTCT |
| | | | CCGGGAGCTGCATGTGTCAGAGGTTTTC |
| | | | ACCGTCATCACCGAAACGCGCGAGGCAG |
| | | | CAGATCAATTCGCGCGCGAAGGCGAAGC |
| | | | GGCATGCATAATGTGCCTGTCAAATGGA |
| | | | CGAAGCAGGGATTCTGCAAACCCTATGC |
| | | | TACTCCGTCAAGCCGTCAATTGTCTGAT |
| | | | TCGTTACCAATTATGACAACTTGACGGC |
| | | | TACATCATTCACTTTTTCTTCACAACCG |
| | | | GCACGGAACTCGCTCGGGCTGGCCCCGG |
| | | | TGCATTTTTTAAATACCCGCGAGAAATA |
| | | | GAGTTGATCGTCAAAACCAACATTGCGA |
| | | | CCGACGGTGGCGATAGGCATCCGGGTGG |
| | | | TGCTCAAAAGCAGCTTCGCCTGGCTGAT |
| | | | ACGTTGGTCCTCGCGCCAGCTTAAGACG |
| | | | CTAATCCCTAACTGCTGGCGGAAAAGAT |
| | | | GTGACAGACGCGACGGCGACAAGCAAAC |
| | | | ATGCTGTGCGACGCTGGCGATATCAAAA |
| | | | TTGCTGTCTGCCAGGTGATCGCTGATGT |
| | | | ACTGACAAGCCTCGCGTACCCGATTATC |
| | | | CATCGGTGGATGGAGCGACTCGTTAATC |
| | | | GCTTCCATGCGCCGCAGTAACAATTGCT |
| | | | CAAGCAGATTTATCGCCAGCAGCTCCGA |
| | | | ATAGCGCCCTTCCCCTTGCCCGGCGTTA |
| | | | ATGATTTGCCCAAACAGGTCGCTGAAAT |
| | | | GCGGCTGGTGCGCTTCATCCGGGCGAAA |
| | | | GAACCCCGTATTGGCAAATATTGACGGC |
| | | | CAGTTAAGCCATTCATGCCAGTAGGCGC |
| | | | GCGGACGAAAGTAAACCCACTGGTGATA |
| | | | CCATTCGCGAGCCTCCGGATGACGACCG |
| | | | TAGTGATGAATCTCTCCTGGCGGGAACA |
| | | | GCAAAATATCACCCGGTCGGCAAACAAA |
| | | | TTCTCGTCCCTGATTTTTCACCACCCCC |
| | | | TGACCGCGAATGGTGAGATTGAGAATAT |
| | | | AACCTTTCATTCCCAGCGGTCGGTCGAT |
| | | | AAAAAAATCGAGATAACCGTTGGCCTCA |
| | | | ATCGGCGTTAAACCCGCCACCAGATGGG |
| | | | CATTAAACGAGTATCCCGGCAGCAGGGG |
| | | | ATCATTTTGCGCTTCAGCCATACTTTTC |
| | | | ATACTCCCGCCATTCAGAGAAGAAACCA |
| | | | ATTGTCCATATTGCATCAGACATTGCCG |
| | | | TCACTGCGTCTTTTACTGGCTCTTCTCG |
| | | | CTAACCAAACCGGTAACCCCGCTTATTA |
| | | | AAAGCATTCTGTAACAAAGCGGGACCAA |
| | | | AGCCATGACAAAAACGCGTAACAAAAGT |
| | | | GTCTATAATCACGGCAGAAAAGTCCACA |
| | | | TTGATTATTTGCACGGCGTCACACTTTG |
| | | | CTATGCCATAGCATTTTTATCCATAAGA |
| | | | TTAGCGGATCCTACCTGACGCTTTTTAT |
| | | | CGCAACTCTCTACTGTTTCTCCATACCC |
| | | | GTTTTTTTGGGCGACCTCGTCGGAGGTT |
| | | | GTATGTCCGGTGTTCCGTGACGTCATCG |
| | | | GGCATTCATCATTCATAGAATGTGTTAC |
| | | | GGAGGAAACAAGTAATGACAAACACTCA |
| | | | AAGTGCATTTTTTATGCCTTCAGTCAAT |
| | | | CTATTTGGTGCAGGATCAGTTAATGAGG |
| | | | TTGGAACTCGATTAGCTGATCTTGGTGT |
| | | | GAAAAAAGCTTTATTAGTTACAGATGCT |
| | | | GGTCTTCACGGTTTAGGTCTTTCTGAAA |
| | | | AAATTTCCAGTATTATTCGTGCAGCTGG |
| | | | TGTGGAAGTATCCATTTTTCCAAAAGCC |
| | | | GAACCAAATCCAACCGATAAAAACGTCG |
| | | | CAGAAGGTTTAGAAGCGTATAACGCTGA |
| | | | AAACTGTGACAGCATTGTCACTCTGGGC |
| | | | GGCGGAAGTTCACATGATGCCGGAAAAG |
| | | | CCATTGCATTAGTAGCTGCTAATGGTGG |
| | | | AAAAATTCACGATTATGAAGGTGTCGAT |
| | | | GTATCAAAAGAACCAATGGTCCCGCTAA |
| | | | TTGCGATTAATACAACAGCTGGTACAGG |
| | | | CAGTGAATTAACTAAATTCACAATCATC |
| | | | ACAGATACTGAACGCAAAGTGAAAATGG |
| | | | CCATTGTGGATAAACATGTAACACCTAC |
| | | | ACTTTCAATCAACGACCCAGAGCTAATG |
| | | | GTTGGAATGCCTCCGTCCTTAACTGCTG |
| | | | CTACTGGATTAGATGCATTAACTCATGC |
| | | | AATTGAAGCATATGTTTCAACTGGTGCT |
| | | | ACTCCAATTACAGATGCACTTGCAATTC |
| | | | AGGCGATCAAAATCATTTCTAAATACTT |
| | | | GCCGCGTGCAGTTGCAAATGGAAAAGAC |
| | | | ATTGAAGCACGTGAACAAATGGCCTTCG |
| | | | CTCAATCATTAGCTGGCATGGCATTCAA |
| | | | TAACGCGGGTTTAGGCTATGTTCATGCG |
| | | | ATTGCACACCAATTAGGAGGATTCTACA |
| | | | ACTTCCCTCATGGCGTTTGCAATGCGGT |
| | | | CCTTCTGCCATATGTATGTCGATTTAAC |
| | | | TTAATTTCTAAAGTGGAACGTTATGCAG |
| | | | AAATCGCTGCTTTTCTTGGTGAAAATGT |
| | | | CGACGGTCTAAGTACGTACGATGCAGCT |
| | | | GAAAAAGCTATTAAAGCGATCGAAAGAA |
| | | | TGGCTAAAGACCTTAACATTCCAAAAGG |
| | | | CTTTAAAGAACTAGGTGCTAAAGAAGAA |
| | | | GACATTGAGACTTTAGCTAAGAATGCGA |
| | | | TGAAAGATGCATGTGCATTAACAAATCC |
| | | | TCGTAAACCTAAGTTAGAAGAAGTCATC |
| | | | CAAATTATTAAAAATGCGATGTAAAAAC |
| | | | CAAAAAGGAACATCGATATGACAACAAA |
| | | | CTTTTTCATTCCACCAGCCAGCGTAATT |
| | | | GGACGCGGTGCAGTAAAGGAAGTAGGAA |
| | | | CAAGACTTAAGCAAATTGGAGCTAAGAA |
| | | | AGCGCTTATCGTTACAGATGCATTCCTT |
| | | | CACAGCACAGGTTTATCTGAAGAAGTTG |
| | | | CTAAAAACATTCGTGAAGCTGGCGTTGA |
| | | | TGTTGCGATTTTCCCAAAAGCTCAACCA |
| | | | GATCCAGCAGATACACAAGTTCATGAAG |
| | | | GTGTAGATGTATTCAAACAAGAAAACTG |
| | | | TGATTCACTTGTTTCTATCGGTGGAGGT |
| | | | AGCTCTCACGATACAGCTAAAGCAATCG |
| | | | GTTTAGTTGCAGCAAACGGCGGAAGAAT |
| | | | CAATGACTATCAAGGTGTAAACAGCGTA |
| | | | GAAAAACCAGTCGTTCCAGTAGTTGCAA |
| | | | TCACTACAACAGCTGGTACTGGTAGTGA |
| | | | AACAACATCTCTTGCGGTTATTACAGAC |
| | | | TCTGCACGTAAAGTAAAAATGCCTGTTA |
| | | | TTGATGAGAAAATTACTCCAACTGTAGC |
| | | | AATTGTTGACCCAGAATTAATGGTGAAA |
| | | | AAACCAGCTGGATTAACAATCGCAACTG |
| | | | GTATGGATGCATTGTCCCATGCAATTGA |
| | | | AGCATATGTTGCAAAAGGTGCTACACCA |
| | | | GTTACTGATGCATTTGCTATTCAAGCAA |
| | | | TGAAACTTATCAATGAATACTTACCAAA |
| | | | AGCGGTTGCGAACGGAGAAGACATCGAA |
| | | | GCACGTGAAAAAATGGCTTATGCACAAT |
| | | | ACATGGCAGGAGTGGCATTTAACAACGG |
| | | | TGGTTTAGGACTAGTTCACTCTATTTCT |
| | | | CACCAAGTAGGTGGAGTTTACAAATTAC |
| | | | AACACGGAATCTGTAACTCAGTTAATAT |
| | | | GCCACACGTTTGCGCATTCAACCTAATT |
| | | | GCTAAAACTGAGCGCTTCGCACACATTG |
| | | | CTGAGCTTTTAGGTGAGAATGTTGCTGG |
| | | | CTTAAGCACTGCAGCAGCTGCTGAGAGA |
| | | | GCAATTGTAGCTCTTGAAAGAATCAACA |
| | | | AATCCTTCGGTATCCCATCTGGCTATGC |
| | | | AGAAATGGGCGTGAAAGAAGAGGATATC |
| | | | GAATTATTAGCGAAAAACGCATACGAAG |
| | | | ACGTATGTACTCAAAGCAACCCACGCGT |
| | | | TCCTACTGTTCAAGACATTGCACAAATC |
| | | | ATCAAAAACGCTATGCATCATCACCATC |
| | | | ACCACTGATAGAGGAACTATTACGGGAG |
| | | | AATGACATGGAACTTCAATTAGCTCTAG |
| | | | ATTTGGTAAACATTGAAGAAGCAAAACA |
| | | | AGTAGTAGCTGAGGTTCAGGAGTATGTC |
| | | | GATATCGTAGAAATCGGTACTCCGGTTA |
| | | | TTAAAATTTGGGGTCTTCAAGCTGTAAA |
| | | | AGCAGTTAAAGACGCATTCCCTCATTTA |
| | | | CAAGTTTTAGCTGACATGAAAACTATGG |
| | | | ATGCTGCAGCATATGAAGTTGCGAAAGC |
| | | | AGCTGAGCATGGCGCTGATATCGTAACA |
| | | | ATTCTTGCAGCAGCTGAAGATGTATCAA |
| | | | TTAAAGGTGCTGTAGAAGAAGCGAAAAA |
| | | | ACTTGGCAAAAAAATCCTTGTTGACATG |
| | | | ATCGCAGTTAAAAATTTAGAAGAGCGTG |
| | | | CAAAACAAGTGGATGAAATGGGCGTAGA |
| | | | CTACATTTGCGTGCACGCTGGATACGAT |
| | | | CTTCAAGCAGTAGGTAAAAACCCATTAG |
| | | | ATGATCTTAAGAGAATTAAAGCTGTCGT |
| | | | GAAAAATGCAAAAACTGCTATTGCGGGC |
| | | | GGAATCAAATTAGAAACATTACCTGAAG |
| | | | TTATCAAAGCAGAACCGGATCTTGTCAT |
| | | | TGTTGGCGGCGGTATTGCTAACCAAACT |
| | | | GATAAAAAAGCAGCAGCTGAAAAAATTA |
| | | | ATAAATTAGTTAAACAAGGGTTATGATC |
| | | | AGCATGCTGACAACTGAATTTTTAGCTG |
| | | | AAATTGTAAAAGAATTAAATAGTTCGGT |
| | | | TAACCAAATCGCCGATGAAGAAGCCGAA |
| | | | GCACTGGTTAACGGAATCCTTCAATCAA |
| | | | AGAAAGTTTTTGTAGCCGGTGCAGGAAG |
| | | | ATCCGGTTTTATGGCTAAATCCTTCGCA |
| | | | ATGCGAATGATGCACATGGGTATTGATG |
| | | | CCTATGTCGTTGGCGAAACCGTAACACC |
| | | | TAACTATGAAAAAGAAGACATCTTAATC |
| | | | ATTGGATCCGGCTCAGGAGAAACAAAAA |
| | | | GTCTCGTTTCCATGGCTCAAAAAGCAAA |
| | | | AAGCATTGGCGGAACCATCGCGGCTGTA |
| | | | ACGATCAACCCTGAATCAACAATTGGGC |
| | | | AATTAGCGGATATCGTTATTAAAATGCC |
| | | | AGGTTCGCCTAAAGATAAATCAGAAGCT |
| | | | AGAGAAACCATCCAACCAATGGGATCTC |
| | | | TTTTTGAACAAACCTTATTATTGTTCTA |
| | | | TGATGCTGTCATTTTGAGATTCATGGAG |
| | | | AAAAAGGGCTTGGATACAAAAACAATGT |
| | | | ACGGAAGACATGCTAATCTTGAGTAGTC |
| | | | CATCTTTCGTAAGGAAATCACCATGATC |
| | | | AAGATTGCACCTTCTATTCTTTCAGCTA |
| | | | ATTTTGCACGACTTGAAGAAGAAATAAA |
| | | | AGATGTTGAACGGGGCGGAGCCGATTAC |
| | | | ATTCATGTTGATGTCATGGATGGTCATT |
| | | | TTGTGCCAAATATAACAATTGGCCCATT |
| | | | AATTGTCGAGGCAATTAGACCTGTCACA |
| | | | AACTTACCTTTAGATGTTCATTTAATGA |
| | | | TAGAAAATCCAGATCAATACATTGGGAC |
| | | | GTTTGCCAAAGCAGGTGCTGATATATTA |
| | | | TCTGTCCATGTTGAAGCTTGTACTCATT |
| | | | TGCACAGAACCATTCAATATATTAAATC |
| | | | TGAAGGTATAAAAGCTGGAGTGGTATTA |
| | | | AACCCTCATACTCCCGTTTCAATGATTG |
| | | | AACATGTAATAGAGGATGTTGATCTTGT |
| | | | ATTGCTTATGACGGTTAATCCTGGCTTT |
| | | | GGGGGACAATCATTCATTCATTCTGTCC |
| | | | TACCTAAAATAAAACAAGTTGCTAACAT |
| | | | CGTAAAAGAGAAAAATTTGCAGGTTGAA |
| | | | ATTGAAGTAGACGGTGGAGTAAATCCTG |
| | | | AAACGGCTAAACTTTGCGTAGAAGCAGG |
| | | | AGCCAATGTCCTTGTTGCAGGTTCAGCC |
| | | | ATATATAATCAAGAGGATAGAAGTCAAG |
| | | | CCATTGCAAAAATTAGAAATTGAACAGG |
| | | | TAAGTTTCCAGGCATCAAATAAAACGAA |
| | | | AGGCTCAGTCGAAAGACTGGGCCTTTCG |
| | | | TTTTATCTGTTGTTTGTCGGTGAACGCT |
| | | | CTCCTGAGTAGGACAAATCCGCCGGGAG |
| | | | CGGATTTGAACGTTGCGAAGCAACGGCC |
| | | | CGGAGGGTGGCGGGCAGGACGCCCGCCA |
| | | | TAAACTGCCAGGCATCAAATTAAGCAGA |
| | | | AGGCCATCCTGACGGATGGCCTTTTTTG |
| | | | ACGGCTAGCTCAGTCCTAGGGATAATGC |
| | | | TAGCACCAGCCTCGAGGGAAACCACGTA |
| | | | AGCTCCGGCGTTTAAACACCCATAACAG |
| | | | ATACGGACTTTCTCAAAGGAGAGTTATC |
| | | | AATGAGGGAATTGAAAAGCGAAAAGCGT |
| | | | GTTCAGTCGTTAGCTATGGAATTTCTCT |
| | | | CTGTAGCACAGCAAGCAGCTCTCGCTTC |
| | | | TTATCCTTGGATAGGAAAAGGTAATAAA |
| | | | AACGAAGTTGATAGGGCTGGTACGGAAG |
| | | | CTATGCGCAATCGACTGAACCTCATTGA |
| | | | TATGAGCGGTTTAATTGTTATTGGTGAA |
| | | | GGGGAAATGGACGAAGCTCCTATGCTTT |
| | | | ATATTGGAGAGGAACTCGGAACAGGAAA |
| | | | AGGACCCCAACTCGATATTGCAGTAGAC |
| | | | CCTGTTGATGGAACGGGTTTAATGGCAA |
| | | | AAGGAATGGATAATTCAATAGCAGTAAT |
| | | | TGCTGCATCCACTAGAGGAAGTTTACTG |
| | | | CATGCCCCAGATATGTACATGGAAAAGA |
| | | | TAGCTGTGGGACCAAAAGCAAAAGGCTG |
| | | | CGTAAATCTAGACGCATCTTTAACAGAA |
| | | | AATATGAAATCAGTTGCTAAAGCTTTAG |
| | | | GGAAAGATTTAAGAGAATTAACTGTAAT |
| | | | GATACAGGATAGACCACGTCATGATCAT |
| | | | TTGATCCAACAAGTAAGAGATGTAGGGG |
| | | | CTAGACTCAAATTATTTTCTGATGGTGA |
| | | | CGTTACAAGGGCAATAGGTACTGCACTC |
| | | | GAAGAAGTAGACGTTGATATATTAGTAG |
| | | | GAACTGGCGGTGCTCCAGAAGGAGTAAT |
| | | | TGCTGCAACCGCACTGAAGTGTTTGGGG |
| | | | GGAGATTTCCAAGGAAGACTTGCTCCTC |
| | | | AAAACGAAGAAGAATTTGATCGCTGTAT |
| | | | TACGATGGGAATAACAGATCCAAGAAAA |
| | | | ATTTTCACAATAGATGAAATTGTAAAAT |
| | | | CAGATGATTGCTTTTTTGTAGCAACAGG |
| | | | AATAACTGACGGACTGCTTATAAATGGT |
| | | | ATTCGAAAAAAAGAAGATGGTTTAATGC |
| | | | AAACGCACTCTTTTCTTACAATTGGAGG |
| | | | AAGCAGCGTAAAATACCAATTTATTGAA |
| | | | GCTTATCATTGATAATAAACGTAATAAA |
| | | | TGACGTTTGATGTATCTAATTGAATGCT |
| | | | CTTTTATGTTGATGTTTCGGAACTGTTT |
| | | | CGGAACCCTCCTTTTTCGGTTAATATTC |
| | | | TCAAAATTCAGTTTTATGTCGCAGTAAC |
| | | | GATTAGCAACTTCAATTAGATATAACGA |
| | | | AGAAAGCGATTTCCCGATCTTATCATGT |
| | | | TAGTTTCCTCAGCTTGAAACTTTCCTGA |
| | | | TTATCCGTAAAGGAAATACACTTGTAAA |
| | | | GCAGATGTTAAAGGAAAAATTTCCCTTT |
| | | | GTTAAGTTGTGAACAAGATGGTATCTCA |
| | | | TCCTTGTCCATCTCTGAATGGCAATAAA |
| | | | TTATTCTTGTGTGACAGTGTGAAAACCT |
| | | | TCGTTTCAGAGATTCATTTTCATGAAAG |
| | | | AACATAGGTGGTAAGAAATCCCCAAAAT |
| | | | GATCCTAAGACCATAATCTAGGGATGTC |
| | | | ACAAAAAGTCAACCCCTATGATAGGATG |
| | | | GTTCAGATATTAGACAGCTTAGCTCTCC |
| | | | ACTATCTGAACGATCCTTTATGAAGTTA |
| | | | TCAAAGAGCAATAAATAAACGGAAAATT |
| | | | ACCTCGAAAGAGGATTCTAAAAATACTA |
| | | | TATAAGGAGTGGGAATTATGCCATTAGT |
| | | | TTCAATGAAGGATATGTTAAATCATGGA |
| | | | AAAGAAAATGGATATGCTGTTGGACAGT |
| | | | TTAACATCAATAATCTTGAGTTTGGTCA |
| | | | AGCGATTTTACAAGCTGCAGAGGAAGAG |
| | | | AAGTCTCCTGTTATTATCGGGGTATCTG |
| | | | TAGGTGCTGCTAATTACATGGGTGGATT |
| | | | TAAGTTAATTGTTGATATGGTCAAATCA |
| | | | TTAATGGATTCATATAACGTAACGGTAC |
| | | | CAGTTGCTATTCATCTTGACCATGGTCC |
| | | | AAGTCTTGAGAAATGTGTACAAGCCATC |
| | | | CATGCTGGATTTACATCTGTTATGATCG |
| | | | ATGGTTCCCATCTTCCACTTGAAGAAAA |
| | | | TATTGAATTAACAAAACGTGTGGTTGAA |
| | | | ATAGCACATTCTGTTGGCGTATCTGTTG |
| | | | AGGCAGAGCTAGGTCGTATCGGTGGACA |
| | | | AGAAGATGATGTAGTAGCTGAATCATTT |
| | | | TATGCTATCCCTTCAGAATGTGAGCAAT |
| | | | TAGTTCGTGAAACAGGAGTAGACTGCTT |
| | | | TGCACCTGCGTTAGGTTCTGTCCATGGT |
| | | | CCGTATAAAGGTGAACCAAAACTTGGTT |
| | | | TTGATCGGATGGAGGAAATTATGAAATT |
| | | | AACAGGTGTTCCTCTTGTTCTCCACGGT |
| | | | GGTACAGGTATTCCAACAAAAGATATTC |
| | | | AAAAAGCTATTTCGCTTGGTACAGCAAA |
| | | | AATTAACGTAAATACAGAAAGCCAAATT |
| | | | GCTGCTACAAAAGCCGTTCGAGAAGTTT |
| | | | TAAATAACGATGCTAAGCTGTTTGATCC |
| | | | TCGCAAATTTTTAGCACCGGCTCGGGAA |
| | | | GCGATTAAAGAAACCATTAAAGGTAAAA |
| | | | TGCGTGAATTTGGATCTTCAGGTAAAGC |
| | | | TTAATAAAAAACAGACATTATGGGAGGG |
| | | | GAAATCGTGCTCCAACAAAAAATAGATA |
| | | | TTGATCAGTTATCCATTCAAACTATTAG |
| | | | AACTCTATCAATTGATGCAATTGAAAAG |
| | | | GTTGGATCAGGCCATCCGGGGATGCCAA |
| | | | TGGGGGCTGCCCCGATGGCCTATACACT |
| | | | TTGGACAAAATTTATGAATTACAATCCA |
| | | | AGCAACCCGAATTGGTTTAATCGTGACC |
| | | | GTTTTGTATTGTCAGCGGGACACGGATC |
| | | | CATGTTATTATACAGCCTATTACATTTA |
| | | | ACTGGTTATGATCTATCATTAGAAGATT |
| | | | TGAAAAACTTCCGCCAATGGGGAAGCAA |
| | | | AACACCTGGTCACCCTGAATTTGGCCAT |
| | | | ACACCTGGGGTTGATGCCACAACAGGTC |
| | | | CGTTAGGGCAAGGTATTGCCATGGCAGT |
| | | | TGGGATGGCGATGGCTGAAAGACATTTA |
| | | | GCGTCTAAATACAATCGTTATAAATTTA |
| | | | ATATTATTGATCACTACACATACAGCAT |
| | | | TTGTGGCGATGGGGACTTGATGGAAGGT |
| | | | GTATCTGCAGAGGCAGCTTCACTTGCAG |
| | | | GGCACCTTAAACTTGGTCGCTTAATTGT |
| | | | ATTATACGATTCAAATGATATTTCTCTT |
| | | | GATGGCGATCTTCATATGTCATTTAGTG |
| | | | AGAGTGTTCAAGATCGTTTTAAAGCATA |
| | | | CGGCTGGCAAGTACTTCGTGTTGAGGAC |
| | | | GGCAATGATATCGATTCAATCGCAAAAG |
| | | | CGATAGCTGAAGCGAAAAACAACGAAGA |
| | | | CCAACCAACATTAATTGAAGTCAAAACA |
| | | | ATAATTGGATACGGCTCACCGAATAAAG |
| | | | GTGGAAAGTCTGATGCGCACGGCTCACC |
| | | | ACTTGGAAAAGAGGAAATAAAGCTTGTA |
| | | | AAAGAACATTACAACTGGAAATATGATG |
| | | | AGGATTTTTATATCCCTGAAGAAGTAAA |
| | | | AGAATATTTTAGAGAATTAAAAGAAGCA |
| | | | GCAGAGAAGAAGGAACAAGCATGGAATG |
| | | | AGTTGTTCGCACAATATAAAGAAGCATA |
| | | | TCCAGCACTTGCAAAGGAATTAGAACAA |
| | | | GCGATTAATGGTGAACTACCAGAAGGCT |
| | | | GGGATGCTGATGTTCCTGTTTACCGTGT |
| | | | CGGAGAAGATAAACTTGCTACTCGTTCT |
| | | | TCCAGTGGTGCAGTGTTAAATGCTCTAG |
| | | | CGAAAAATGTTCCGCAACTACTTGGCGG |
| | | | TTCTGCGGATTTAGCTTCATCTAATAAA |
| | | | ACGCTACTAAAAGGGGAAGCAAATTTCA |
| | | | GCGCTACAGATTATAGCGGACGTAATAT |
| | | | TTGGTTTGGTGTTCGTGAATTTGGAATG |
| | | | GGTGCTGCTGTCAACGGAATGGCCCTAC |
| | | | ACGGTGGTGTAAAAGTATTTGGAGCAAC |
| | | | ATTCTTTGTATTCTCTGATTATTTACGT |
| | | | CCGGCCATTCGTCTCTCAGCATTAATGA |
| | | | AACTACCAGTTATTTATGTCTTTACACA |
| | | | TGATAGCGTTGCTGTAGGTGAAGATGGA |
| | | | CCAACACATGAACCAATTGAACAATTGG |
| | | | CATCCTTACGTGCAATGCCTGGTATCTC |
| | | | TACAATTCGCCCGGCTGATGGCAATGAG |
| | | | ACAGCTGCAGCTTGGAAGTTGGCGTTAG |
| | | | AAAGTAAAGACGAACCAACAGCTCTTAT |
| | | | CCTCTCACGTCAAGACTTACCAACACTT |
| | | | GTTGATTCTGAAAAAGCGTATGAGGGTG |
| | | | TTAAAAAAGGTGCATATGTGATCTCTGA |
| | | | AGCAAAAGGTGAAGTTGCTGGTTTGTTA |
| | | | TTAGCATCTGGTTCTGAAGTTGCTTTAG |
| | | | CTGTTGAAGCACAAGCAGCGCTGGAAAA |
| | | | GGAAGGTATTTATGTTTCAGTTGTTAGT |
| | | | ATGCCTAGCTGGGATCGTTTTGAAAAAC |
| | | | AATCTGATGCATACAAAGAAAGTGTACT |
| | | | TCCAAAAAACGTAAAAGCACGTCTTGGT |
| | | | ATTGAAATGGGGGCTTCCTTAGGTTGGA |
| | | | GTAAATATGTTGGTGATAACGGTAACGT |
| | | | CCTCGCCATTGATCAATTTGGATCCTCA |
| | | | GCACCAGGAGATAAAATAATTGAAGAAT |
| | | | ACGGTTTTACAGTCGAAAATGTCGTTTC |
| | | | TCATTTTAAAAAGCTTCTCTAAAAGTCT |
| | | | TGCCCTTGTTTAATCGGCTGTTTTGGCA |
| | | | CTGGAGATCTTGTACAGGAATAGTTCAT |
| | | | AATTTCTGAAAGCAAGCTCCGATAGTGT |
| | | | TTAGCATTTTTTTGAATAAATCCACAGA |
| | | | AGAGTTCAAGACGGCACTTTCCTCTCAA |
| | | | ACGAAATCAAAGCAAACGGTACAATGTG |
| | | | CGCAACTTTTGATGTAGGAAAATGGCCT |
| | | | GTGCCAATTCTGAATTGGTCTCTCACCA |
| | | | ATTTTTTAGCCGTTCTTATTCTTCTGTG |
| | | | AAATCCTCACAAAGTGTTTTACAAACAA |
| | | | CTTCCTATCTTGTTTTAAAGCAGCGAGT |
| | | | GACTGTTCTTTACTGAACTTCACCTGAA |
| | | | CCTACTTGCGAACCTCTTCCAACGCTTT |
| | | | GATGAACGATTGAATCATCATGGAACTC |
| | | | ATCAATGATCACTAACACAATGCGGTAA |
| | | | GGCGGTTTCAATCGCTGTTTTGTATGCT |
| | | | TTCCACATATTGATCACAATTCGATGAG |
| | | | GCGTTCAGGATGAGATATTGTTTCAAGA |
| | | | TCTCAATAACGTCCTCTTTTTTTTTCTG |
| | | | TTTTTGTTCTGTTTTTCTTTTTCAACTT |
| | | | CTTTTTCTCAAACTTGAAAAAGTAAACA |
| | | | AACAAGAGATTATTATCAGAAAATTCGT |
| | | | TCATTTATAGAAGATATGGAGGAAAACA |
| | | | AGAATGAATAAGATTGCAGTATTAACTA |
| | | | GCGGCGGGGATGCACCAGGAATGAACGC |
| | | | TGCTATTCGTGCGGTCGTTCGAAGAGGA |
| | | | ATCTTTAAAGGACTAGATGTTTATGGTG |
| | | | TAAAAAATGGCTACAAAGGTTTAATGAA |
| | | | TGGGAATTTTGTTTCAATGAACCTCGGA |
| | | | AGTGTGGGTGATATTATTCACCGAGGAG |
| | | | GCACTATCTTACAAACTACACGCTGTAA |
| | | | AGAGTTTAAGACAGCTGAAGGGCAACAA |
| | | | CAGGCTTTAGCACAGCTAAAAAAAGAAG |
| | | | GCATTGATGGCTTAATCGTGATTGGTGG |
| | | | AGATGGCACTTTTGAAGGTGCGAGAAAA |
| | | | TTAACTGCCCAAGAGTTTCCAACTATTG |
| | | | GTATTCCGGCAACCATTGACAATGACAT |
| | | | TGCAGGGACGGAATATACAATTGGATTT |
| | | | GATACTGCTGTGAACACAGCAGTGGAAG |
| | | | CAATTGATAAAATTCGTGATACGGCAGC |
| | | | CTCTCATGATCGTATCTATGTCGTTGAA |
| | | | GTAATGGGCCGCAATGCAGGAGACATCG |
| | | | CTCTATGGGCAGGAATGTGTGCGGGAGC |
| | | | AGAATCAATTATTATCCCAGAAGCCGAC |
| | | | CATGATGTGGAAGATGTAATTGATCGTA |
| | | | TTAAACAAGGATATCAGCGAGGAAAAAC |
| | | | GCACAGTATTATTGTGGTTGCAGAAGGG |
| | | | GCATTTAATGGAGTAGGAGCAATAGAAA |
| | | | TTGGTAGAGCAATTAAAGAGAAAACAGG |
| | | | ATTTGACACAAAGGTAACCATACTTGGG |
| | | | CATATTCAACGTGGGGGATCTCCTAGCG |
| | | | CTTACGACCGAATGATGAGCAGTCAGAT |
| | | | GGGTGCAAAAGCCGTGGATTTGCTGGTT |
| | | | GAAGGCAAAAAAGGTCTGATGGTAGGAT |
| | | | TAAAAAATGGTCAACTGATTCATACACC |
| | | | TTTTGAGGAAGCTGCGAAAGATAAGCAT |
| | | | ACGGTTGATTTGTCCATCTACCATTTAG |
| | | | CAAGAAGTCTTTCTTTATAGACCGGGGA |
| | | | AGTACCGTGACCACCGAGCAGTTCCCGC |
| | | | CCCAATTCCTGCGTGAAATGATCGAGCA |
| | | | GCTGGACGCCAGCATCCAGGAGCTCGCA |
| | | | CGCAAGGAAAAGGGACTTGCGGCATCCC |
| | | | TGGGCACGGGCCGGGTCGCCGAGCTCAA |
| | | | GGAATACTGGGACCACGTTGTTACAACC |
| | | | AATTAACCAATTCTGATTAGAAAAACTC |
| | | | ATCGAGCATCAAATGAAACTGCAATTTA |
| | | | TTCATATCAGGATTATCAATACCATATT |
| | | | TTTGAAAAAGCCGTTTCTGTAATGAAGG |
| | | | AGAAAACTCACCGAGGCAGTTCCATAGG |
| | | | ATGGCAAGATCCTGGTATCGGTCTGCGA |
| | | | TTCCGACTCGTCCAACATCAATACAACC |
| | | | TATTAATTTCCCCTCGTCAAAAATAAGG |
| | | | TTATCAAGTGAGAAATCACCATGAGTGA |
| | | | CGACTGAATCCGGTGAGAATGGCAAAAG |
| | | | CTTATGCATTTCTTTCCAGACTTGTTCA |
| | | | ACAGGCCAGCCATTACGCTCGTCATCAA |
| | | | AATCACTCGCATCAACCAAACCGTTATT |
| | | | CATTCGTGATTGCGCCTGAGCGAGACGA |
| | | | AATACGCGATCGCTGTTAAAAGGACAAT |
| | | | TACAAACAGGAATCGAATGCAACCGGCG |
| | | | CAGGAACACTGCCAGCGCATCAACAATA |
| | | | TTTTCACCTGAATCAGGATATTCTTCTA |
| | | | ATACCTGGAATGCTGTTTTCCCGGGGAT |
| | | | CGCAGTGGTGAGTAACCATGCATCATCA |
| | | | GGAGTACGGATAAAATGCTTGATGGTCG |
| | | | GAAGAGGCATAAATTCCGTCAGCCAGTT |
| | | | TAGTCTGACCATCTCATCTGTAACATCA |
| | | | TTGGCAACGCTACCTTTGCCATGTTTCA |
| | | | GAAACAACTCTGGCGCATCGGGCTTCCC |
| | | | ATACAATCGATAGATTGTCGCACCTGAT |
| | | | TGCCCGACATTATCGCGAGCCCATTTAT |
| | | | ACCCATATAAATCAGCATCCATGTTGGA |
| | | | ATTTAATCGCGGCCTCGAGCAAGACGTT |
| | | | TCCCGTTGAATATGGCTCATAACACCCC |
| | | | TTGTATTACTGTTTATGTAAGCAGACAG |
| | | | TTTTATTGTTCATGATGATATATTTTTA |
| | | | TCTTGTGCAATGTAACATCAGAGATTTT |
| | | | GAGACACAACGTGGCTTTGTTGAATAAA |
| | | | TCGAACTTTTGCTGAGTTGAAGGATCAG |
| | | | ATCACGCATCTTCCCGACAACGCAGACC |
| | | | GTTCCGTGGCAAAGCAAAAGTTCAAAAT |
| | | | CACCAACTGGTCCACCTACAACAAAGCT |
| | | | CTCATCAACCGTGGCTCCCTCACTTTCT |
| | | | GGCTGGATGATGGGGCGATTCAGGCCTG |
| | | | GTATGAGTCAGCAACACCTTCTTCACGA |
| | | | GGCAGACCTCAGCGCTAGCGGAGTGTAT |
| | | | ACTGGCTTACTATGTTGGCACTGATGAG |
| | | | GGTGTCAGTGAAGTGCTTCATGTGGCAG |
| | | | GAGAAAAAAGGCTGCACCGGTGCGTCAG |
| | | | CAGAATATGTGATACAGGATATATTCCG |
| | | | CTTCCTCGCTCACTGACTCGCTACGCTC |
| | | | GGTCGTTCGACTGCGGCGAGCGGAAATG |
| | | | GCTTACGAACGGGGCGGAGATTTCCTGG |
| | | | AAGATGCCAGGAAGATACTTAACAGGGA |
| | | | AGTGAGAGGGCCGCGGCAAAGCCGTTTT |
| | | | TCCATAGGCTCCGCCCCCCTGACAAGCA |
| | | | TCACGAAATCTGACGCTCAAATCAGTGG |
| | | | TGGCGAAACCCGACAGGACTATAAAGAT |
| | | | ACCAGGCGTTTCCCCCTGGCGGCTCCCT |
| | | | CGTGCGCTCTCCTGTTCCTGCCTTTCGG |
| | | | TTTACCGGTGTCATTCCGCTGTTATGGC |
| | | | CGCGTTTGTCTCATTCCACGCCTGACAC |
| | | | TCAGTTCCGGGTAGGCAGTTCGCTCCAA |
| | | | GCTGGACTGTATGCACGAACCCCCCGTT |
| | | | CAGTCCGACCGCTGCGCCTTATCCGGTA |
| | | | ACTATCGTCTTGAGTCCAACCCGGAAAG |
| | | | ACATGCAAAAGCACCACTGGCAGCAGCC |
| | | | ACTGGTAATTGATTTAGAGGAGTTAGTC |
| | | | TTGAAGTCATGCGCCGGTTAAGGCTAAA |
| | | | CTGAAAGGACAAGTTTTGGTGACTGCGC |
| | | | TCCTCCAAGCCAGTTACCTCGGTTCAAA |
| | | | GAGTTGGTAGCTCAGAGAACCTTCGAAA |
| | | | AACCGCCCTGCAAGGCGGTTTTTTCGTT |
| | | | TTCAGAGCAAGAGATTACGCGCAGACCA |
| | | | AAACGATCTCAAGAAGATCATCTTATTA |
| | | | AGGGGTCTGACGCTCAGTGGAACGAAAA |
| | | | CTCACGTTAAGGGATTTTGGTCATGAGA |
| | | | TTATCAAAAAGGATCTTCACCTAGATCC |
| | | | TTTTAAATTAAAAATGAAGTTTTAAATC |
| | | | AATCTAAAGTATATATGAGTAAACTTGG |
| | | | TCTGACAGGTGAGCTGATACCGCTCGCC |
| | | | GCATGCACATGCAGTCATGTCGTGC |

### G. Examples

### Example 1: Conversion of Methanol into 3-Hydroxypropionate Using an Engineered Microorganism

3-hydroxypropionate (3HP) was produced from a methanol feedstock via the fermentation of an engineered strain of Escherichia coli. Plasmid pNH243 (SEQ ID NO:35) was designed to contain the malonyl-CoA reductase (mcr) from Chloroflexus aurantiacus in two parts (see Liu et al., “Functional balance between enzymes in malonyl-CoA pathway for 3-hydroxypropionate biosynthesis”, Metabolic Engineering, 2016, Vol. 34., pp. 104-111, a copy of which is incorporated by reference herein including any drawings). The plasmid backbone was derived from a commercially available vector (pMAL-5×-HIS, available from New England Biolabs, Ipswitch, Mass.) to contain the pMB1 origin, CarbR resistance, and the Ptac promoter. The mcr gene was split into two fragments, with three mutations added, as described by Liu et al. These two genes were ordered from a commercial vendor (IDT DNA Technologies, Coralville, Iowa) and cloned into holding vectors. These vectors were sequenced and then used as templates for PCR. The PCR fragments were purified and cloned into the vector via Gibson cloning (New England Biolabs). Colonies were screened by PCR and sequenced. One sequence-verified clone was designated as pNH243.

Plasmid pNH241 (SEQ ID NO:34) was designed to contain the accABCD genes from E. coli, overexpressed from a p15a-KanR plasmid backbone and a pBAD promoter. DNA encoding the genes was amplified from E. coli genomic DNA, gel-purified, and assembled with Phusion polymerase to generate a 3.7 kb fragment encoding a synthetic accABCD operon. This was Gibson-cloned into a vector backbone containing the p15a origin and the gene that confers resistance to kanamycin. The resulting reaction was transformed into electrocompetent cells and plated on LB agar supplemented with kanamycin (50 μg/mL). Colonies were screened by PCR and sequenced. One sequence-verified clone was designated as pNH241.

Plasmid pLC130 (SEQ ID NO:37) was constructed to express the mdh2, hps, and phi genes from Bacillus methanolicus MGA3. The genes were amplified from genomic DNA or plasmid pBM19 and cloned on a vector with a CloDF origin and the gene that confers resistance to spectinomycin.

Plasmid pBZ27 (SEQ ID NO:39) was constructed to express the mdh, mdh2, hps, phi, rpeP, glpXP, fbaP, tktP, and pfkP genes from Bacillus methanolicus MGA3. The genes were amplified from genomic DNA or plasmid pBM19 and Gibson-cloned into a vector with a p15a origin and a gene that confers resistance to kanamycin.

Three strains were constructed that decreased the ability for E. coli to oxidize formaldehyde using its endogenous formaldehyde-detoxification pathway. The deletions should each increase the concentration of formaldehyde inside the cells and thus increase flux through HPS-PHI into central metabolism. MC1061 and BW25113 are standard laboratory strains of Escherichia coli. LC23 is MC1061 with gshA deleted; LC476 is MC1061 with frmA deleted. LC474 is BW25113 with frmA deleted. These strains were constructed using lambda-red homologous recombination. (Datsenko and Wanner, “One-step inactivation of chromosomal genes in Escherichia coli K-12 using PCR products”, PNAS vol. 97, issue 12, p. 6640-5 (2000)).

pNH241, pNH243, pLC130, and pBZ27 were transformed into either LC23, LC476, or LC474 and grown on LB plates supplemented with the appropriate antibiotics to identify transformants. Single colonies were picked for subsequent analysis.

### Bioconversion Description

3-hydroxypropionate bioconversions were performed as follows: single colonies of each strain were inoculated into 2 mL of LB supplemented with appropriate antibiotics overnight at 37° C. with shaking at 280 rpm. From these cultures, 500 μL was transferred into 4.5 mL of fresh LB supplemented with appropriate antibiotics. Arabinose was added to a final concentration of 1 mM to induce expression of the genes and these cultures were incubated at 37° C., shaking at 280 rpm. After 3-5 hours, the cultures were centrifuged at 4000 rpm for 5 min, resuspended in phosphate buffer solution (PBS) to wash the cells and centrifuged again. The pellets were resuspended in PBS supplemented with arabinose (1 mM) or PBS supplemented with arabinose (1 mM) and 5 mM ribose, and either unlabeled or ¹³C-labeled methanol in sealed tubes to a final OD600>1. After 2-3 days incubation at 37° C., the cultures were centrifuged and the supernatant was sent to the QB3 Central California 900 MHz NMR facility for analysis or to the Proteomics and Mass Spectrometry Lab at the Danforth Center at the University of Washington at St. Louis for LC-MS analysis.

### Analytical METHODS

1H NMR spectra were collected at the QB3 Central California 900 MHz NMR Facility at 25° C. on a Bruker Biospin Avance II 900 MHz spectrometer equipped with a CPTCI cryoprobe. 32 μL of 8.3 mM sodium 3-(trimethylsilyl)tetradeuteriopropionate (TSP) was added to 500 μL sample as a reference standard to give a final concentration of 0.5 mM. Spectra were referenced to TSP (0 ppm) and concentration of metabolites was calculated by relative peak integration compared to TSP (9H), correcting for sample dilution by the reference standard. ¹³C isotopic enrichment was determined from the splitting of ¹³C-attached protons. The percent enrichment was calculated as the ¹³C-split peak areas divided by the total peak integration for ¹²C- and ¹³C-attachedprotons: C2 of 3-hydroxypropionate (¹²C: t, 2.44 ppm; ¹³C: t, 2.37 and 2.51 ppm).

The samples for liquid chromatography-mass spectrometry (LC-MS) were filtered and then used without further preparation. One microliter of each sample was injected onto a 0.5×100 mm Proteomix SAX column using 25% methanol (A) and 250 mM (NH₄)₂CO₃ (B) attached to a Q-Exactive mass spectrometer. Data were recorded in negative ion mode from m/z 80-250 at a resolution setting of 70,000 (FWHM at m/z 200). Integrated areas for 3-hydroxypropionic acid and its isotopologues were extracted using the QuanBrowser application of Xcalibur. ¹³C isotopologues areas were reported for the 3-hydroxypropionic acid. In order to determine the contribution of the methanol to the ¹³C-labeled 3HP, the peak areas for 3HP were analyzed (separately quantified for unlabeled, singly-labeled, doubly-labeled, and triply-labeled carbons) from feeding either unlabeled methanol or ¹³C-labeled methanol and subtracted the former (as a baseline control) from the latter (in which the labeled methanol contributes to the labeling of the product). The resulting values correspond to the contribution of the labeled methanol to the different isotopologues of 3HP, as shown in the table below.

The quantities of 3HP were measured using NMR for certain strains in PBS supplemented with 0.5% ¹³C-methanol and ribose (5 mM final concentration), where noted in the TABLE 1 below. The concentration of ¹³C-3-hydroxyproproinate reported is a sum of all ¹³C-labeled 3-hydroxyproproinate species. ND indicates “Not Detected.” The data show that methanol is converted into 3-hydroxyproproinate in various strain backgrounds and fermentation conditions.

TABLE 1

| Base | | | 13C-3HP |
|---|---|---|---|
| strain | Plasmids | Media | (mM) |
| LC23 | pBZ27, pNH243 | PBS ribose + | 0.15 |
| | | 13C—MeOH (0.5%) | |
| LC23 | pBZ27, pNH243 | PBS ribose + | ND |
| | | unlabeled MeOH (0.5%) | |
| LC23 | pLC130, pNH241, | PBS ribose + | 0.16 |
| | pNH243 | 13C—MeOH (0.5%) | |
| LC23 | pLC130, pNH241, | PBS ribose + | ND |
| | pNH243 | unlabeled MeOH (0.5%) | |
| LC23 | pLC130, pNH241, | PBS + | 0.1 |
| | pNH243 | 13C—MeOH (0.5%) | |
| LC23 | pLC130, pNH241, | PBS + | ND |
| | pNH243 | unlabeled MeOH (0.5%) | |
| LC474 | pLC130, pNH241, | PBS + | 0.06 |
| | pNH243 | 13C—MeOH (0.5%) | |
| LC474 | pLC130, pNH241, | PBS + | ND |
| | pNH243 | unlabeled MeOH (0.5%) | |
| LC474 | pLC130, pNH241, | PBS ribose + | 0.18 |
| | pNH243 | 13C—MeOH (0.5%) | |
| LC474 | pLC130, pNH241, | PBS ribose + | ND |
| | pNH243 | unlabeled MeOH (0.5%) | |

Using LC-MS, labeled 3-hydroxypropionate species were measured and quantified for two strains incubated in PBS supplemented with arabinose (1 mM) and ¹³C-methanol (4% v/v), as shown in the Table 2 below. The data show that a significant fraction of 3-hydroxyproproinate produced is made from methanol, and that some strains produce 3-hydroxypropionate in which all three carbon atoms present are derived from methanol.

Using LC-MS, ¹³C-labeled cellular metabolites were measured

TABLE 2

| | | Number of carbons in which 3HP labeled with 13C | | | |
|---|---|---|---|---|---|
| | | | | | Total |
| Base | | 1 | 2 | 3 | (at least one |
| strain | Plasmids | carbon | carbons | carbons | carbon labeled) |
| LC23 | pBZ27, | 9% | 14% | 5% | 29% |
| | pNH243 | | | | |
| LC23 | pLC130, | 23% | 6% | 0% | 29% |
| | pNH241, | | | | |
| | pNH243 | | | | |

### Example 2: Conversion of Methane into 3-Hydroxypropionate Using an Engineered E. coli

Two engineered strains of E. coli were cultured in order to convert methane, a low-cost feedstock, into 3-hydroxypropionate, a valuable intermediate chemical. One strain converts the methane into methanol, while the second strain converts methanol into 3-hydroxypropionate. Each strain is grown up to a suitable density, the expression of the proteins in the engineered pathways is induced, and the two strains are combined into a single, sealed vial. Methane is injected into the headspace in the vial. After a suitable period of time, a sample of the liquid is removed from the vial and injected into a gas chromatography-mass spectrometry (GC-MS) system for analysis.

One of the two strains is an E. coli strain that expresses a methane monooxygenase enzyme that converts methane into methanol. This strain (NH784) was derived from the commercially-available strain NEB Express (New England Biolabs, Ipswich, Mass.) in two steps. First, the operon araBAD was deleted from its chromosomal locus by replacement with a gene that confers resistance to chloramphenicol (cat), using the method of Datsenko and Wanner (Datsenko and Wanner, “One-step inactivation of chromosomal genes in Escherichia coli K-12 using PCR products”, PNAS vol. 97, issue 12, p. 6640-5 (2000), which is incorporated by reference herein, including any drawings). Next the strain was transformed with the plasmid pNH265 (SEQ ID NO:36) via electroporation, recovery in SOC, and growth overnight on LB agar plates supplemented with 100 μs/mL of spectinomycin. The plasmid pNH265 was constructed by standard molecular biology cloning techniques, combining a cloning vector with both PCR-amplified genomic DNA fragments and synthetic DNA.

The second strain is an E. coli strain that expresses a pathway to convert methanol into 3-hydroxypropionate. Several variants of this strain were tested and found to be capable of conversion of methanol into 3-hydroxypropionate. All variants were comprised of three plasmids: pNH241 (SEQ ID NO:34), pNH243 (SEQ ID NO:35), and either pLC130 (SEQ ID NO:37) or pLC158 (SEQ ID NO:38) (see Table 3). Plasmids pLC130 and pLC158 both comprise a spectinomycin-resistance gene, an origin of replication, and an arabinose-inducible promoter driving three genes required for assimilation of methanol into the ribulose monophosphate (RuMP) cycle (methanol dehydrogenase (MDH), 3-hexulose-6-phosphate synthase (HPS), and 6-phospho-3-hexuloisomerase (PHI)). Plasmid pLC130 comprises the methanol dehydrogenase from Bacillus methanolicus, while pLC158 comprises the methanol dehydrogenase from Corynebacterium glutamicum.

Both HPS and PHI genes were derived from Bacillus methanolicus. The sequences of all the plasmids are provided herein. The background strains of the six variants also differed (see TABLE 4). All these E. coli strains were derived from either BW25113 or MC1061, which are widely available laboratory strains. These strains also had deletions of the genes frmA and glpK, and some strains had deletion of the gene gnd. The gene glpK was deleted from the three base strains to prevent growth using glycerol as a carbon source. Other methods of generating reducing equivalents for the methane oxidation step are possible, including expression of NADH-producing formate dehydrogenase, such as fdh from Candida boidinii, and including formate in the media. The deletions were made using homologous recombination. Strain genotypes were confirmed by colony PCR, and failed to grow in minimal media with glycerol as the sole carbon source.

Combinations of the three plasmids were transformed sequentially into each base strain. Strains with all three plasmids were selected on LB plates supplemented with 50 μg/mL spectinomycin, 50 μg/mL carbenicillin and 25 μg/mL kanamycin. Single colonies were picked for fermentations.

TABLE 3

| Plasmid | SEQ | | |
|---|---|---|---|
| Name | ID NO: | Components | Purpose |
| pLC130 | 37 | pBAD-MDH-HPS-PHI | Methanol |
| | | (B. methanolicus) | assimilation |
| pLC158 | 38 | pBAD-MDH | Methanol |
| | | (C. glutamicum)- | assimilation |
| | | HPS-PHI | |
| | | (B. methanolicus) | |
| pNH241 | 34 | pBAD-accDACB | Malonyl-CoA |
| | | (E. coli) | overproduction |
| pNH243 | 35 | pTAC-MCR_{C}-MCR_{N} | 3HP production |
| | | (C. aurantiacus) | |
| pNH265 | 36 | pBAD-MMO; | Methane |
| | | constitutive groES- | monooxygenase |
| | | groEL2- groES-groEL | |

TABLE 4
Strains used in this study

| Strain Name | Base Strain | Plasmid(s) | Components |
|---|---|---|---|
| LC474 | BW25113 | | |
| | ΔfrmA-FRT | | |
| LC527 | MC1061 | | |
| | ΔfrmA-FRT | | |
| | Δgnd-FRT | | |
| LC476 | MC1061 | | |
| | ΔfrmA-FRT | | |
| NH283 | NEB Express | | |
| | ΔaraBAD::cat | | |
| LC631 | LC474 ΔglpK- | pLC130 + | Methanol-assimilation, |
| | FRT | pNH241 + | 3HP production |
| | | pNH243 | |
| LC632 | LC527 ΔglpK- | pLC130 + | Methanol-assimilation, |
| | FRT | pNH241 + | 3HP production |
| | | pNH243 | |
| LC633 | LC476 ΔglpK- | pLC130 + | Methanol-assimilation, |
| | FRT | pNH241 + | 3HP production |
| | | pNH243 | |
| LC634 | LC474 ΔglpK- | pLC158 + | Methanol-assimilation, |
| | FRT | pNH241 + | 3HP production |
| | | pNH243 | |
| LC635 | LC527 ΔglpK- | pLC158 + | Methanol-assimilation, |
| | FRT | pNH241 + | 3HP production |
| | | pNH243 | |
| LC636 | LC476 ΔglpK- | pLC158 + | Methanol-assimilation, |
| | FRT | pNH241 + | 3HP production |
| | | pNH243 | |
| NH784 | NH283 | pNH265 | Methane monooxygenase |

Strains were cultured in standard media and induced in separate tubes. NH784 was grown overnight to stationary phase at 37° C. After 16 hours, a new culture was inoculated using 1 mL of the overnight culture into 10 mL of LB supplemented with 100 μg/mL spectinomycin, 1 mM L-arabinose, 50 μM ferric citrate, and 200 μM L-cysteine. Cells were divided evenly between two 50 mL conical tubes, which were shaken at 30° C. for 4 hours and 30 minutes.

Strains LC631-LC636 were grown overnight to stationary phase in LB supplemented with 50 μs/mL carbenicillin, 25 μg/mL kanamycin, 50 μs/mL spectinomycin. After 16 hours, a new culture was inoculated using 0.5 mL of the overnight cultures into 5 mL of LB supplemented with 50 μs/mL carbenicillin, 25 μs/mL kanamycin, 50 μs/mL spectinomycin, 1 mM L-arabinose, 1 mM IPTG. Cells were shaken at 37° C. in 50 mL conical tubes for 4 hours and 30 minutes, with 5 mM ribose added for the last 90 minutes.

At the end of induction, cells were washed in phosphate buffered saline (PBS), and resuspended in PBS supplemented with 1 mM L-arabinose, 1 mM IPTG, 50 μM ferric citrate, 200 μM L-cysteine, and 0.4% glycerol to a final OD600 of 5.

240 μL of NH784 was mixed with 240 μL of each of strains LC631-636. Each of these 6 mixtures was split evenly between two glass vials, yielding 12 vials total. These vials were sealed with rubber stoppers. Using a syringe, 1 mL of ¹³C-labeled methane was injected into the headspace above the liquid in one of the vial of each pair, while 1 mL of unlabeled methane was injected into the second vial of each pair. All vials were incubated at 37° C., shaking at 280 rpm. After 70 hours, the samples were centrifuged and the supernatant of each was split into two different tubes, for replicate measurement. These samples were analysed for ¹³C-labeled 3HP acid by GC-MS.

Samples were analysed by The Proteomics & Mass Spectrometry Facility at the Danforth Plant Science Center. 50 μL of each sample was added to a tube and dried. To the dry samples, 25 μL MBSTFA was added and allowed to react for one hour at 70° C. with shaking After the samples cooled, 25 μL hexane was added. One microliter was injected for each sample. The data were integrated then searched against the NIST spectral database for identification. The integrated peak heights were calculated for each relevant peak. Since 3-hydroxypropionate contains 3 carbon atoms, each of which may be ¹²C or ¹³C, it is possible to observe ¹³C-methane incorporation into each position of 3-hydroxypropionate. As such, molecules of 3HP may contain one, two, or three ¹³C atoms. Due to the difference in the molecular mass, these molecules can be quantified by GC-MS, since they appear as separate peaks in the spectrum.

Normalizing to total 3HP in each sample gives us the fraction of the 3HP that is singly-, doubly- or triply-¹³C-labeled. Below are the data from six different strains, each of which is in a co-culture with NH784.

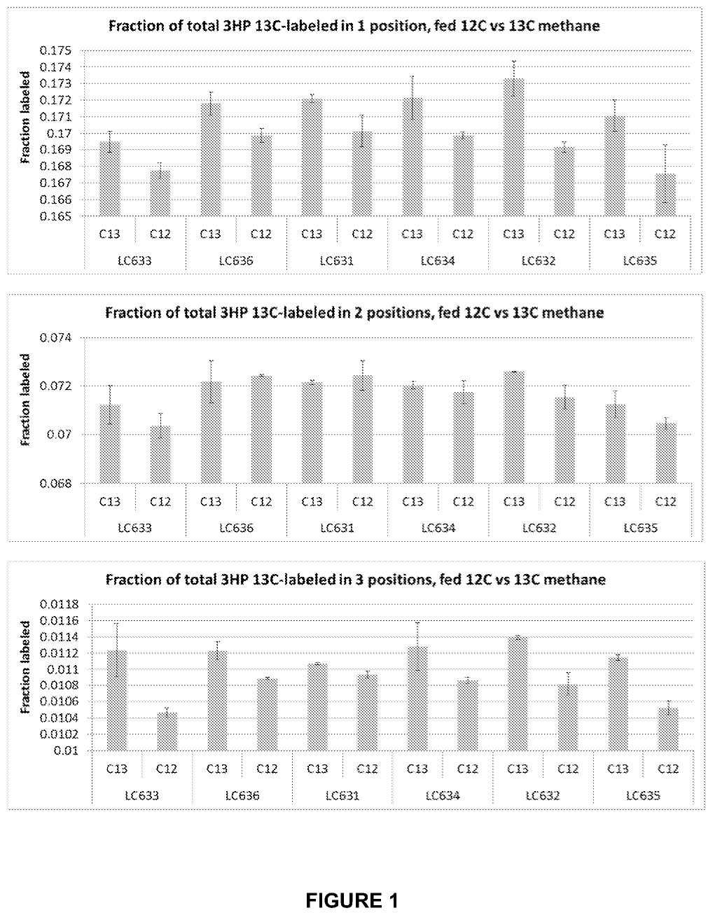

FIG. 1 depicts 6 co-culture experiments where the culture was split into two vials and the headspace was injected with unlabeled or ¹³C-methane. The fraction of total 3-hydroxypropionate that is ¹³C-labeled is plotted for each of the 12 vials. The top panel shows the fraction of 3-hydroxypropionate that is singly-¹³C-labeled. The middle panel shows the fraction of 3-hydroxypropionate that is doubly-¹³C-labeled. The bottom panel shows the fraction of 3-hydroxypropionate that is triply-¹³C-labeled.

These data show that a significant fraction of 3-hydroxypropionate produced is made from methane, and that some strains produce 3HP in which all three carbon atoms present are derived from methane.

All publications, patents, and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A synthetic culture comprising one or more microorganisms comprising one or more modifications that improve the production of a product from a substrate, wherein the substrate comprises methane and/or methanol.

2. The synthetic culture according to claim 1, wherein the substrate comprises methane.

3. The synthetic culture according to claim 2, wherein the product comprises 3-hydroxyproprionate.

4. The synthetic culture according to claim 1, wherein the product comprises 3-hydroxyproprionate.

5. The synthetic culture according to claim 1, wherein the product comprises a substance derived from acetyl-CoA and/or malonyl-CoA.

6. The synthetic culture according to claim 1, wherein at least one of the one or more microorganisms comprises Escherichia coli.

7. The synthetic culture according to claim 1, wherein the one or more microorganisms comprises a first at least one microorganism and a second at least one microorganism, wherein the first at least one microorganism produces methanol from methane and the second at least one microorganism produces 3-hydroxypropionate from methanol.

8. The synthetic culture according to claim 1, wherein the one or more modifications comprise exogenous polynucleotides or deletion of one or more genes.

9. The synthetic culture according to claim 8, wherein the exogenous polynucleotides encode polypeptides selected from one or more polypeptides comprising methane monooxygenase (EC 1.14.13.25), malonyl-CoA reductase (EC 1.2.1.75), acetyl-CoA carboxylase (EC 6.4.1.2), methanol dehydrogenase (EC 1.1.1.244 or EC 1.1.2.7), 3-hexulose-6-phosphate synthase (EC 4.1.2.43), and/or 6-phospho-3-hexuloisomerase (EC 5.3.1.27).

10. The synthetic culture according to claim 9, wherein the methanol dehydrogenase comprises a methanol dehydrogenase from Bacillus methanolicus, Bacillus stearothermophilus, and/or Corynebacterium glutamicum.

11. The synthetic culture according to claim 9, wherein the acetyl-CoA carboxylase comprises accABCD from Escherichia coli.

12. The synthetic culture according to claim 9, wherein the methane monooxygenase comprises the soluble methane monooxygenase from Methylococcus capsulatus (Bath).

13. The synthetic culture according to claim 9, wherein the malonyl-CoA reductase comprises a malonyl-CoA reductase from Chloroflexus aurantiacus.

14. (canceled)

15. The synthetic culture according to claim 9, wherein the malonyl-CoA reductase has one or more substitutions.

16. The synthetic culture according to claim 14, wherein the one or more substitutions comprise N940V, K1106W, and/or S1114R.

17. The synthetic culture according to claim 1, wherein the one or more modifications comprise at least one exogenous polynucleotide comprising one or more of rpeP, glpXP, fbaP, tktP, and/or pfkP genes from Bacillus methanolicus.

18. The synthetic culture according to claim 1, wherein the one or more modifications comprise deletion of glpK, frmA, pgi, gnd, gshA, and/or lrp.

19. The synthetic culture according to claim 8, wherein the exogenous polynucleotides comprise one more of more nucleic acids comprising one or more sequences comprising one or more of SEQ ID NOs: 34-39.

20. The synthetic culture according to claim 9, wherein the one or more one or more polypeptides comprise polypeptides having one or more amino acid sequences comprising one or more sequences set forth in any one or more of SEQ ID NOs: 1-33.

21. The synthetic culture according to claim 9, wherein the one or more polypeptides comprise polypeptides having one or more amino acid sequences comprising one or more sequences that are about 95% identical to one or more of the sequences set forth in SEQ ID NOs: 1-33.

22. A method for producing a product, comprising culturing the synthetic culture according to claim 1 under suitable culture conditions and for a sufficient period of time to produce the product.
